(19) United States
(12) Patent Application Publication
(10) Pub. No.: US 2023/0357286 A1
(43) Pub. Date: Nov. 9, 2023

(54) KETONE SYNTHESIS AND APPLICATIONS

(71) Applicant: President and Fellows of Harvard College, Cambridge, MA (US)

(72) Inventors: Yoshito Kishi, Cambridge, MA (US); Atsushi Umehara, Cambridge, MA (US)

(73) Assignee: President and Fellows of Harvard College, Cambridge, MA (US)

(21) Appl. No.: 18/017,557

(22) PCT Filed: Jul. 24, 2020
(86) PCT No.: PCT/US2020/043501

§ 371 Date: Jan. 23, 2023

(51) Int. Cl. B01J 31/18; C07F 7/18; C07F 15/04

(52) U.S. Cl. CPC C07F 7/1892; B01J 31/1815; C07F 15/045; B01J 2231/4205; B01J 2531/847

(57) ABSTRACT

Provided are new nickel./zirconium-mediated coupling reactions useful in the synthesis of ketone-containing compounds, e.g., halichondrin natural products and related molecules. A feature of the present disclosure is the use of a nickel(I) catalyst in tandem with a nickel (II) catalyst in the Ni/Zr-mediated coupling reactions. Without wishing to be bound by any particular theory, the nickel (I) catalyst selectively activates the electrophilic coupling partner (i.e., the compound of Formula (A)), and the nickel(ll) catalyst selectively activates the nucleophilic coupling partner (i.e., a thioester of Formula (B)). This dual catalyst system leads to improved coupling efficiency and eliminates the need for a large excess of one of the coupling partners (i.e., a compound of Formula (A) or (B)).

### BACKGROUND OF THE INVENTION

Halichondrins are polyether natural products, originally isolated from the marine scavenger Halichondria okadai by Uemura, Hirata, and coworkers. See, e.g., Uemura, D.; Takahashi, K.; Yamamoto, T.; Katayama, C.; Tanaka, J.; Okumura, Y.; Hirata, Y. J. Am. Chem. Soc. 1985, 107, 4796; Hirata, Y.; Uemura, D. Pure Appl. Chem. 1986, 58, 701. Several additional members, including halistatin, were isolated from various marine scavengers. This class of natural products displays interesting structural diversity, such as the oxidation state of the carbons of the C8-C14 polycycle, and the length of the carbon backbone. Thus, this class of natural products is sub-grouped into the norhalichondrin series (e.g., norhalichondrin A, B, and C), the halichondrin series (e.g., halichondrin A, B, C), and the homohalichondrin series (e.g., homohalichondrin A, B, C) (see FIG. 1). Except halichondrin A, all the members have been isolated from natural sources. Due to their intriguing structural architecture and extraordinary antitumor activity, halichondrins have received much attention from the scientific community.

### SUMMARY OF THE INVENTION

Described herein are new nickel/zirconium-mediated coupling reactions useful in the synthesis of ketone-containing compounds, e.g., halichondrin natural products and related molecules. As described herein, a feature of the present disclosure is the use of a nickel(I) catalyst in tandem with a nickel(II) catalyst in the Ni/Zr-mediated coupling reactions. Without wishing to be bound by a particular theory, the nickel(I) catalyst selectively activates the electrophilic coupling partner (i.e., the compound of Formula (A)), and the nickel(II) catalyst selectively activates the nucleophilic coupling partner (i.e., a thioester of Formula (B)). In certain embodiments, this dual catalyst system leads to improved coupling efficiency and eliminates the need for an excess of one of the coupling partners (i.e., a compound of Formula (A) or (B)). This is particularly advantageous in reactions involving synthetically complex coupling partners, such as those used in the synthesis of complex natural products.

In one aspect, the present disclosure provides methods for preparing ketones using a Ni/Zr-mediated coupling reaction, as outlined in Scheme 1A. These coupling reactions can be applied to the synthesis of halichondrins (e.g., halichondrin A, B, C; homohalichondrin A, B, C; norhalichondrin A, B, C), and analogs thereof. The coupling reactions described herein can also be applied to the synthesis of compounds described in, e.g., U.S. Publication No. 2017/0137437, published May 18, 2017; International Publication No. WO 2016/003975, published Jan. 7, 2016; U.S. Publication No. 2018/0230164, published Aug. 16, 2018; International Publication No. WO 2016/176560, published Nov. 3, 2016; U.S. Publication No. 2018/0155361, published Jun. 7, 2018; International Publication No. WO 2018/187331, published Oct. 11, 2018; U.S. Pat. No. 9,938,288, issued Apr. 10, 2018; International Publication No. WO 2019/009956, published Jan. 10, 2019; International Publication No. WO 2019/099646, published May 23, 2019; and International Publication No. 2019/010363, published Jan. 10, 2019; the entire contents of each of which is incorporated herein by reference.

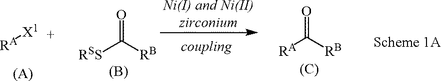

Application of Ni/Zr-mediated coupling reactions provided herein to the preparation of compounds in the halichondrin series (e.g., halichondrin A, B, C, and analogs thereof) is outlined in Scheme 2A, for example. This strategy involves coupling of a “left half” building block with a “right half” building block via a Ni/Zr-mediated coupling reaction described herein.

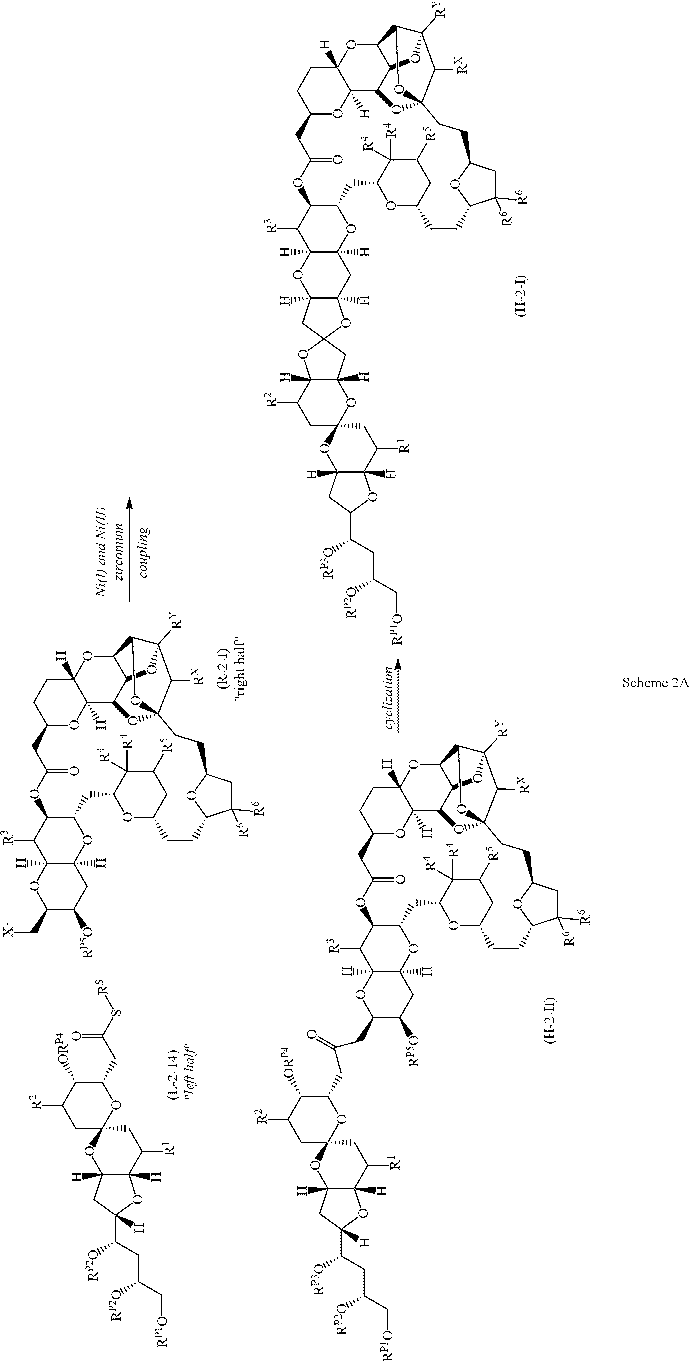

Application of Ni/Zr-mediated coupling reactions provided herein to the preparation of compounds in the homohalichondrin series (e.g., homohalichondrin A, B, C, and analogs thereof) is outlined in Scheme 2B, for example. This strategy involves coupling of a “left half” building block with a “right half” building block via a Ni/Zr-mediated coupling reaction described herein.

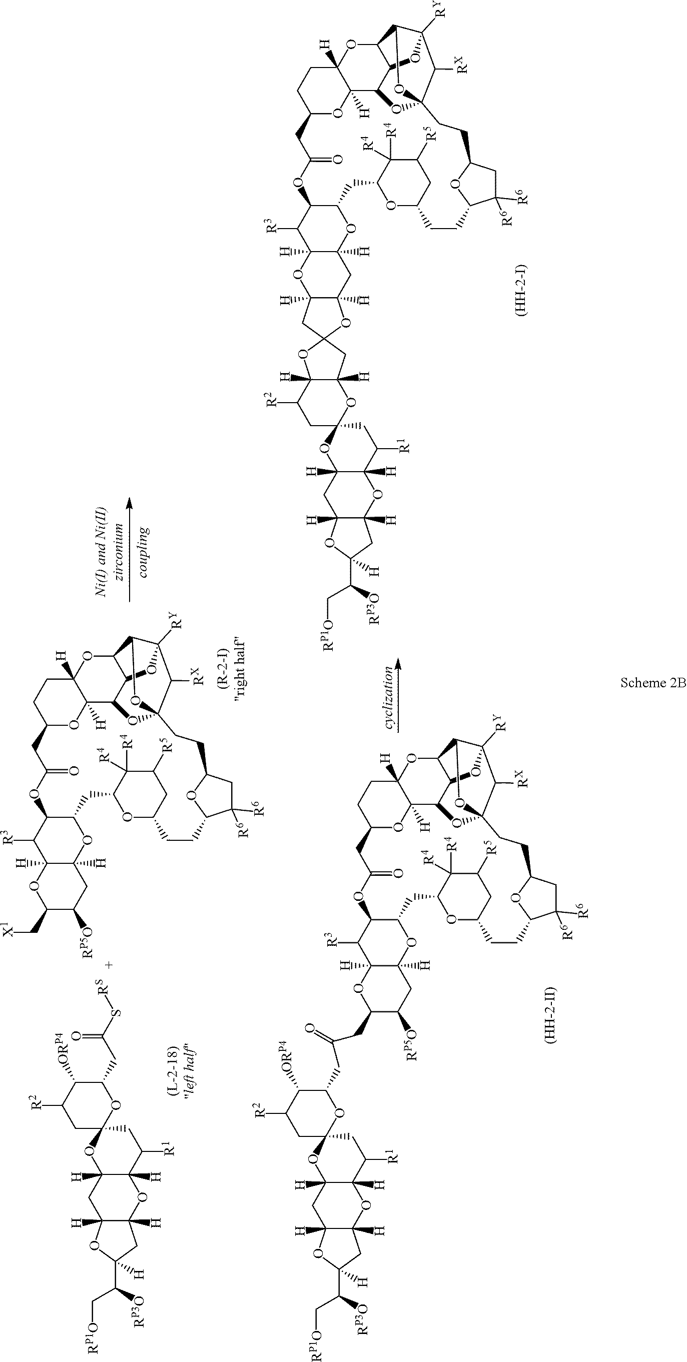

Application of Ni/Zr-mediated coupling reactions provided herein to the preparation of compounds in the norhalichondrin series (e.g., norhalichondrin A, B, C, and analogs thereof) is outlined in Scheme 2C, for example. This strategy involves coupling of a “left half” building block with a “right half” building block via a Ni/Zr-mediated coupling reaction described herein.

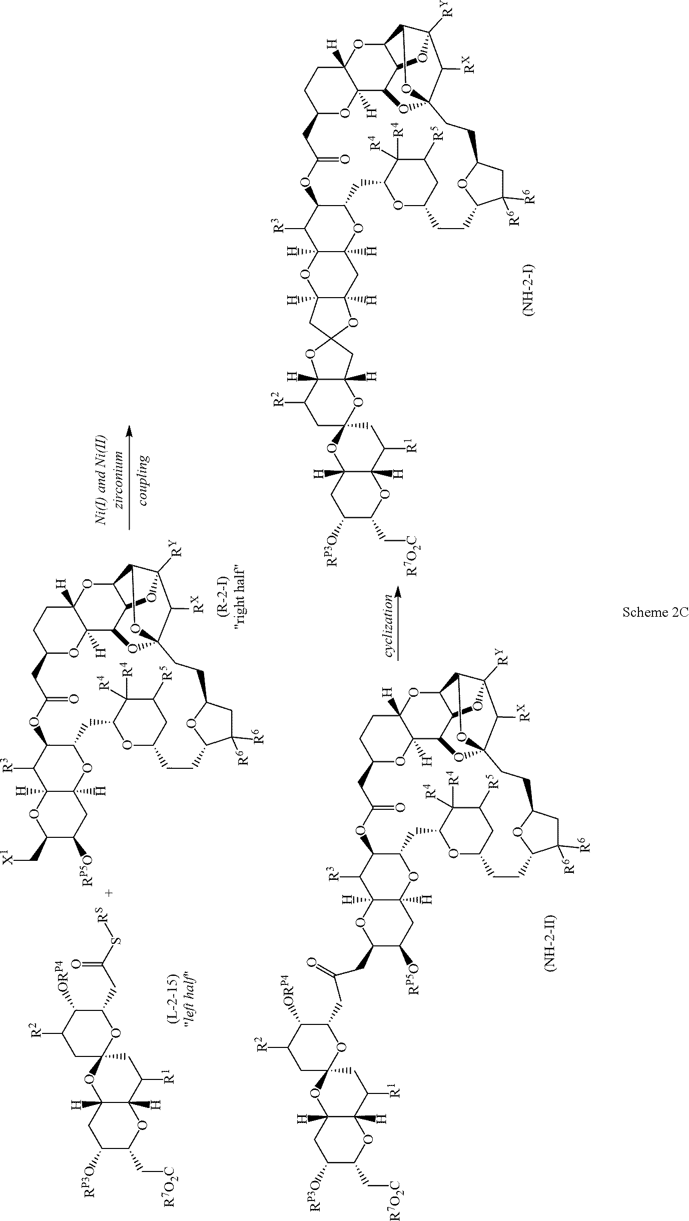

Application of Ni/Zr-mediated coupling reactions provided herein to the preparation of additional halichondrin analogs is outlined in Scheme 2D, for example. This strategy involves coupling of a “left half” building block with a “right half” building block via a Ni/Zr-mediated coupling reaction described herein. Compounds of Formula (H3-2-I) are described in, e.g., International Publication No. WO 2019/099646, published May 23, 2019, the entire contents of which is incorporated herein by reference. Compounds of Formula (H3-2-I) are also useful as synthetic intermediates in the synthesis of compounds described in, e.g., International Publication Nos. WO 2019/010363, published Jan. 10, 2019; WO 2018/187331, published Oct. 11, 2018; and WO 2019/099646, published May 23, 2019; the entire contents of each of which is incorporated herein by reference.

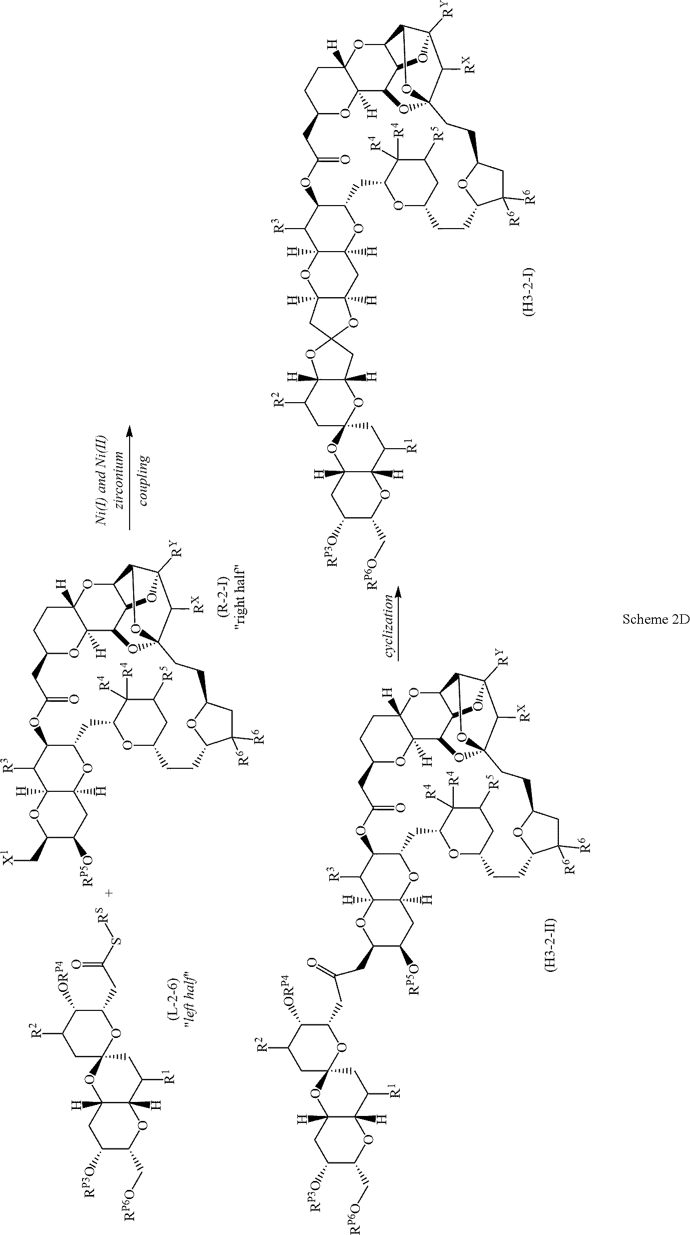

The present disclosure also provides compounds (i.e., intermediates) useful in the methods provided herein. In certain embodiments, the compounds provided herein are useful as synthetic intermediates en route to halichondrins and analogs thereof. All compounds described herein are included as emodiments of the invention. Furthermore, the present disclosure provides reagents and catalysts useful in the methods described herein. All reagents and catalysts described herein are included as embodiments of the invention.

The present disclosure also provides reaction mixtures comprising one or more compounds, reagents, catalysts, and/or solvents described herein. All reaction mixtures described herein are included as embodiments of the invention. The present disclosure also provides kits comprising one or more reagents, catalysts, and/or compounds described herein.

The details of certain embodiments of the invention are set forth in the Detailed Description of Certain Embodiments, as described below. Other features, objects, and advantages of the invention will be apparent from the Definitions, Examples, Figures, and Claims.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March’s Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E.L. Stereochemistry of Carbon Compounds (McGraw-Hill, NY, 1962); and Wilen, S.H., Tables of ResolvingAgents and Optical Resolutions p. 268 (E.L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g.. n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., -CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., -CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include -CHF₂, -CH₂F, -CF₃, -CH₂CF₃, -CF₂CF₃, -CF₂CF₂CF₃, -CC1₃, —CFC1₂, -CF₂C1, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁-₉ alkyl”). In some embodiments, a heteroalkyl group is a saturated group having 1 to 8 carbon atoms and 1 or more heteroatoms within the parent chain (“heteroC₁-₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁-₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂-₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C=C double bond for which the stereochemistry is not specified (e.g., -CH=CHCH₃ or

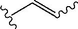

) may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂-₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₃₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7 tetrahydro-1H-pyrrolo[2,3-b]pyridinyl, 4,5,6,7-tetrahydrofum[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroanyl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —ORªª, —ON(R^{bb}]2, —N(R^{bb})2, —N(R^{bb})3⁺X⁻, —N(OR^{cc})R^{bb}, —SH, —SRªª, —SSR^{cc}, —C(═O)Rªª, —CO₂H, —CHO, —C(OR^{cc})₃, —CO₂Rªª, —OC(═O)Rªª, —OCO₂Rªª, —C(═O)N(R^{bb})₂, —OC(═O)N(R^{bb})2, —NR^{bb}C(═O)Rªª, —NR^{bb}CO₂Rªª, —NR^{bb}C(═O)N(R^{bb})2, —C(═NR^{bb})Rªª —C(═NR^{bb})ORªª, —OC(═NR^{bb})Rªª, —OC(═NR^{bb})ORªª, —C(═NR^{bb})N(R^{bb})₂, —OC(═NR^{bb})N(R^{bb} )₂, —NR^{bb}C(═NR^{bb})N(R^{bb})₂. —C(═O)NR^{bb}SO₂Rªª, —NR^{bb}SO₂Rªª, —SO₂N(R^{bb})₂, —SO₂Rªª, —SO₂ORªª, —OSO₂Rªª, —S(═O)Rªª, —OS(═O)Rªª, —Si(R^{aa})₃, —OSi(Rªª)₃ —C(═S)N(R^{bb})₂, —C(═O)SRªª, —C(═S)SRªª, —SC(═S)SRªª, —SC(═O)SRªª, —OC(═O)SRªª, —SC(═O)ORªª, —SC(═O)Rªª, —P(═O)(Rªª)₂, —P(═O)(O^{cc})₂, —OP(═O)(Rªª)₂, —OP(═O)(OR^{cc})₂, —P(═O)(N(R^{bb})₂)₂, —OP(═O)(N(R^{bb})₂)₂, —NR^{bb}P(═O)(Rªª)₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(O^{cc})₄, —OP(R^{cc})₂, —OP(^{cc})₃⁺X⁻, —OP(OR^{cc})₂, —OP(OR^{cc})₃⁺X⁻, —OP(R^{cc})₄, —OP(OR^{cc})₄, —B(Rªª)₂, —B(OR^{cc})₂, —BRªª(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group =O, =S, ═NN(R^{bb})₂, =NNR^{bb}C(=O)Rªª, =NNR^{bb}C(=O)ORªª, =NNR^{bb}S(=0)₂Rªª, ═NR^{bb}, or =NOR^{cc};
each instance of Rªª is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two Rªª groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, -ORªª, -N(R^{cc})₂, —CN, —C(═O)Rªª, —C(═O)N(R^{cc})₂, —CO₂Rªª, —SO₂Rªª, —C(═NR^{cc})ORªª, —C(═NR^{cc})N(R^{cc})₂, -SO₂N(R^{cc})₂, -SO₂R^{cc}, -SO₂OR^{cc}, -SORªª, —C(═S)N(R^{cc})₂, —C(═O)SR^{cc}, —C(═S)SR^{cc}, —P(═O)(Rªª)₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, -SSR^{ee}, —C(═O)R^{ee}, —CO₂H, —CO₂R^{ee}, —OC(═O)R^{ee}, -OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee} —OC(═NR^{ff})R^{ee}, —OC(═NR^{ff}F)OR^{ee}, —C(═NR^{ff})N(R^{ff})₂, —OC(═NR^{ff})N(R^{ff})₂. —NeC(═NR^{ff})N(R^{ff})₂, —NR^{ff}SO₂R^{ee}, —SO₂N(R^{ff})₂, —SO₂R^{ee}, —SO₂OR^{ee}, -OSO₂R^{ee}, —S(═O)R^{ee}, —Si(R^{ee})₃, —OSi(R^{ee})3, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂. —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form =O or =S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, and
each instance of R^{gg} is, independently, halogen, —CN, —NO₂, —N₃, —SO₂H, —SO₃H, —OH, -OC₁₋₆ alkyl, -ON(C₁₋₆ alkyl)₂, -N(C₁₋₆ alkyl)₂, -N(C₁₋₆ alkyl)₃⁺X⁻, -NH(C₁₋₆ alkyl)₂⁺X⁻, -NH₂(C₁₋₆ alkyl)⁺X⁻, —NH₃⁺X⁻, -N(OC₁₋₆ alkyl)(C₁₋₆ alkyl), -N(OH)(C₁₋₆ alkyl), —NH(OH), —SH, -SC₁₋₆ alkyl, -SS(C₁₋₆ alkyl), -C(=O)(C₁₋₆ alkyl), —CO₂H, -CO₂(C₁₋₆ alkyl), —M(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form =O or =S; wherein X⁻ is a counterion.

In certain embodiments, carbon atom substituents include: halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═OX C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal substituents can be joined to form =O or =S; wherein X⁻ is a counterion.

The term “halo” or “halogen” refers to fluorine (fluoro, -F), chlorine (chloro, -C1), bromine (bromo, -Br), or iodine (iodo, -I).

The term “hydroxyl” or “hydroxy” refers to the group -OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)Rªª, —OCO₂Rªª, —OC(═O)N(R^{bb})₂, —OC(═NR_{bb})Rªª, —OC(═NRS)ORªª, —OC(═NR^{bb})N(R^{bb})₂, —OS(═O)Rªª, —OSO₂Rªª, —OSi(Rªª)₃, —OP(R^{cc})₂, —OP(R^{cc})₃⁺X⁻, —OP(OR^{cc})₂, —OP(OR^{cc)}₃⁺X⁻, —OP(═O)(Rªª)₂, —OP(═O)(OR^{cc})₂, and —OP(═O)(N(R^{bb} )₂)₂, wherein X⁻. Rªª, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)Rªª, —NHCO₂Rªª, —NHC(═O)N(R^{bb})₂, —NHC(═NR^{bb})N(R^{bb})₂, —NHSO₂Rªª, —NHP(═O)(OR^{cc})₂, and —NHP(═O)(N(R^{bb})₂)₂, wherein Rªª, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂Rªª, —NR^{bb}C(═O)N(R^{bb})₂, —NR^{bb}C(═NR^{bb})N(R^{bb})₂, —NR^{bb}SO₂Rªª, —NR^{bb}P(═O)(OR^{cc})₂, and —NRb^{b}P(═O)(N(R^{bb})2)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X-, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, -C(=O)OR^{X1}, —C(═O)—O—C(═O)R^{X1}, —C(═O)SR^{x1}, —C(═O)N(R^{X1})₂, —C(═S)R^{X1}, —C(═S)N(R^{x1})₂, —C(═S)O(R^{X1}), —C(═S)S(R^{X1}), —C(═NR^{X1})RX^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di- aliphaticamino, mono- or di- heteroaliphaticamino, mono- or di- alkylamino, mono- or di- heteroalkylamino, mono- or di-arylamino, or mono- or di-heteroarylamino; or two R^{X1} groups taken together form a 5- to 6-membered heterocyclic ring. Exemplary acyl groups include aldehydes (-CHO), carboxylic acids (-CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, eg., a group selected from ketones (e.g., —C(═O)R^{aa}), carboxylic acids (e.g., —CO₂H), aldehydes (-CHO), esters (e.g., —CO₂Rªª, —C(═O)SR^{aa}, —C(═S)SR^{aa}), amides (e.g, —C(═O)N(R^{bb})₂, —C(═O)NR^{bb}SO₂R^{aa}, —C(═S)N(R^{bb})2), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})3, wherein R^{aa} is as defined herein.

The term “oxo” refers to the group =O, and the term “thiooxo” refers to the group =S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, -ORªª, -N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, -SO₂N(R^{cc})₂, -SO₂R^{cc}, -SO₂OR^{cc}, -SOR^{aa}, —C(═S)N(R^{cc})₂, —C(═O)SR^{cc}, —C(═S)SR^{cc}, —P(═O)OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein Rªª, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, -N(R^{cc})₂, —C(═O)R^{aa}, —C(═O)N(R^{cc})2, —CO₂Rªª, —SO₂Rªª, —C(═NR^{cc})R^{aa}, —C(═NR^{cc})ORªª, —C(═NR^{cc})N(R^{cc})₂, -SO₂N(R^{cc})₂, -SO₂R^{cc}, -SO₂OR^{cc}, -SOR^{aa}, —C(═S)N(R^{cc})2, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-( 10,10-dioxo- 10, 10, 10, 1 0-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂Rªª, —C(═O)N(R^{bb})₂, —C(═NR^{bb})R^{aa}, —C(═NR^{bb})ORªª, —C(═NR^{bb})N(R^{bb})₂, —S(═O)R^{aa}, —SO₂R^{aa}, —Si(R^{aa})₃, -P(R^{cc})₂, -P(R^{cc})3⁺X⁻, -P(OR^{cc})₂, -P(OR^{cc})₃⁺X⁻, —P(═O)(R^{aa})₂, —P(═O)(OR^{cc})₂, and —P(═O)(N(R^{bb})₂)₂, wherein X⁻, Rªª, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB, MPM), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)Rªª, —SO₂R^{aa}, -Si(Rªª)₃, -P(R^{cc})₂, -P(R^{cc})₃⁺X⁻, -P(OR^{cc})₂, -P(OR^{cc})₃⁺X⁻, —P(═O)(R^{aa})₂, —P(═O)(OR^{cc})₂, and —P(═O)(N(R^{bb})₂)₂, wherein Rªª, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-butyl, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂- or (HCB₁₁Me₅Br₆)⁻). Exemplary counterions which may be multivalent include CO₃²⁻, HPO₄²⁻, PO₄³⁻. B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, -OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, -OSO₂R^{aa}, -OP(R^{cc})₂, -OP(R^{cc})₃, —OP(═O)₂R^{aa}, —OP(═O)(R^{aa}), —OP(═O)(OR^{cc})₂, —OP(═O)₂N(R^{bb})₂, and -OP(=OXNR^{bb})_{2,} wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The following definitions are more general terms used throughout the present application.

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (-)-isomers respectively). A chiral compound can exist as either individual enantiomer or as a mixture thereof. A mixture containing equal proportions of the enantiomers is called a “racemic mixture”.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)).

The term “catalysis,” “catalyze,” or “catalytic” refers to the increase in rate of a chemical reaction due to the participation of a substance called a “catalyst.” In certain embodiments, the amount and nature of a catalyst remains essentially unchanged during a reaction. In certain embodiments, a catalyst is regenerated, or the nature of a catalyst is essentially restored after a reaction. A catalyst may participate in multiple chemical transformations. The effect of a catalyst may vary due to the presence of other substances known as inhibitors or poisons (which reduce the catalytic activity) or promoters (which increase the activity). Catalyzed reactions have lower activation energy (rate-limiting free energy of activation) than the corresponding uncatalyzed reaction, resulting in a higher reaction rate at the same temperature. Catalysts may affect the reaction environment favorably, bind to the reagents to polarize bonds, form specific intermediates that are not typically produced by a uncatalyzed reaction, or cause dissociation of reagents to reactive forms.

The term “solvent” refers to a substance that dissolves one or more solutes, resulting in a solution. A solvent may serve as a medium for any reaction or transformation described herein. The solvent may dissolve one or more reactants or reagents in a reaction mixture. The solvent may facilitate the mixing of one or more reagents or reactants in a reaction mixture. The solvent may also serve to increase or decrease the rate of a reaction relative to the reaction in a different solvent. Solvents can be polar or non-polar, protic or aprotic. Common organic solvents useful in the methods described herein include, but are not limited to, acetone, acetonitrile, benzene, benzonitrile, 1-butanol, 2-butanone, butyl acetate, tert-butyl methyl ether, carbon disulfide carbon tetrachloride, chlorobenzene, 1-chlorobutane, chloroform, cyclohexane, cyclopentane, 1,2-dichlorobenzene, 1,2-dichloroethane, dichloromethane (DCM), N,N-dimethylacetamide N,N-dimethylformamide (DMF), 1,3-dimethyl-3,4,5,6-tetrahydro-2-pyrimidinone (DMPU), 1,4-dioxane, 1,3-dioxane, diethylether, 2-ethoxyethyl ether, ethyl acetate, ethyl alcohol, ethylene glycol, dimethyl ether, heptane, n-hexane, hexanes, hexamethylphosphoramide (HMPA), 2-methoxyethanol, 2-methoxyethyl acetate, methyl alcohol, 2-methylbutane, 4-methyl-2-pentanone, 2-methyl-1-propanol, 2-methyl-2-propanol, 1-methyl-2-pyrrolidinone, dimethylsulfoxide (DMSO), nitromethane, 1-octanol, pentane, 3-pentanone, 1-propanol, 2-propanol, pyridine, tetrachloroethylene, tetrahyrdofuran (THF), 2-methyltetrahydrofuran, toluene, trichlorobenzene, 1,1,2-trichlorotrifluoroethane, 2,2,4-trimethylpentane, trimethylamine, triethylamine, N,N-diisopropylethylamine, diisopropylamine, water, o-xylene, and p-xylene.

### BRIEF DESCRIPTION OR THE DRAWINGS

The accompanying drawings, which constitute a part of this specification, illustrate several embodiments of the invention and together with the description, serve to explain the principles of the invention.

FIG. 1 shows a ketone coupling used for model studies.

FIG. 2 shows representative bidentate- and tridentate-ligands, and (Me)₃tpy-Ni^{I}I-and py-(Me)imid·Ni^{II}Cl₂-catalysts.

FIG. 3 shows a proposed catalytic cycle of Ni^{I}.

FIG. 4 shows a proposed catalytic cycle of Ni^{II}.

FIG. 5 shows a proposed role of Cp₂Zr^{IV}Cl₂.

FIG. 6 shows a use of the new ketone coupling in the synthesis of halichondrin analogs.

### DETAILED DESCRIPTION OF CERTAIN EMBODIMENTS

Provided herein are Ni/Zr-mediated coupling reactions useful in the preparation of ketone-containing compounds. As described herein, a feature of the present disclosure is the use of a nickel(I) catalyst in tandem with a nickel(II) catalyst in the Ni/Zr-mediated coupling reactions. Without wishing to be bound by a particular theory, the nickel(I) catalyst selectively activates the electrophilic coupling partner (i.e., the compound of Formula (A)), and the nickel(II) catalyst selectively activates the nucleophilic coupling partner (i.e., a thioester of Formula (B)). In certain embodiments, this dual catalyst system leads to improved coupling efficiency and eliminates the need for a large excess of one of the coupling partners (i.e., a compound of Formula (A) or (B)). The Ni/Zr-mediated coupling reactions provided herein are therefore particularly useful for reactions involving complex coupling partners, e.g., in the synthesis of complex natural products such as halichondrins and analogs thereof.

Therefore, also provided herein are methods for the preparation of halichondrins (e.g., halichondrin A, B, C; homohalichondrin A, B, C; norhalichondrin A, B, C) and analogs thereof. For example, in certain embodiments, methods provided herein are useful in the synthesis of compounds described in, e.g., International Publication Nos. WO 2019/010363, published Jan. 10, 2019; WO 2018/187331, published Oct. 11, 2018; and WO 2019/099646, published May 23, 2019; the entire contents of each of which is incorporated herein by reference.

The present disclosure also provides compounds (i.e., intermediates) useful in the methods provided herein. In certain embodiments, the compounds provided herein are useful as synthetic intermediates en route to halichondrins and analogs thereof. All compounds described herein are included as emodiments of the invention. Furthermore, the present disclosure provides reagents and catalysts useful in the methods described herein. All reagents and catalysts described herein are included as embodiments of the invention.

The present disclosure also provides reaction mixtures comprising one or more compounds, reagents, catalysts, and/or solvents described herein. All reaction mixtures described herein are included as embodiments of the invention. The present disclosure also provides kits comprising one or more reagents, catalysts, and/or compounds described herein.

### Ni/Zr-Mediated Coupling Reactions

In one aspect, provided herein are nickel/zirconium-mediated coupling reactions (“Ni/Zr-mediated coupling reactions”) involving coupling of a thioester and an alkyl halide (e.g., alkyl iodide, alkyl bromide, alkyl chloride, etc.) or alkyl-leaving group (e.g., alkyl sulfonate) (Scheme 1A).

The coupling reactions may be intermolecular or intramolecular (i.e., in Scheme 1A, R^{A} and R^{B} are optionally joined by a linker). In certain embodiments, the compound of Formula (A) is a primary or secondary alkyl halide (X¹ = halogen), and the compound of Formula (B) is an alkyl thioester (R^{B} = optionally substituted alkyl), as shown in Scheme 1B.

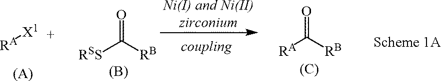

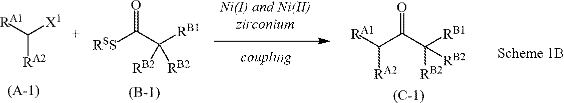

As represented in Scheme 1A, provided herein are methods for preparing a compound of Formula (C):

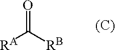

or a salt thereof, the methods comprising reacting a compound of Formula (A):

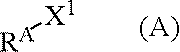

or a salt thereof, with a compound of Formula (B):

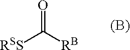

or a salt thereof, in the presence of a nickel(I) complex, a nickel(II) complex, and a zirconium complex; wherein:
R^{A} is optionally substituted alkyl;
R^{B} is optionally substituted alkyl, optionally substituted alkenyl, optionally substituted alkynyl, optionally substituted aryl, optionally substituted carbocyclyl, optionally substituted heteroaryl, or optionally substituted heterocyclyl;
optionally wherein R^{A} and R^{B} are joined together via a linker, wherein the linker is selected from the group consisting of optionally substituted alkylene, optionally substituted heteroalkylene, optionally substituted alkenylene, optionally substituted heteroalkenylene, optionally substituted alkynylene, optionally substituted heteroalkynylene, optionally substituted arylene, optionally substituted heteroarylene, optionally substituted carbocyclylene, optionally substituted heterocyclylene, optionally substituted acylene, and combinations thereof,
X¹ is halogen or a leaving group; and
R^{S} is optionally substituted alkyl, optionally substituted carbocyclyl, optionally substituted aryl, optionally substituted heterocyclyl, or optionally substituted heteroaryl.

In certain embodiments, R^{A} is a small molecule or part of a small molecule. In certain embodiments, R^{B} is a small molecule or part of a small molecule. Small molecules encompass complex small molecules, such as natural products, pharmaceutical agents, and fragments thereof, and intermediates thereto.

As generally defined herein, a “linker” is a group comprising optionally substituted alkylene, optionally substituted heteroalkylene, optionally substituted alkenylene, optionally substituted heteroalkenylene, optionally substituted alkynylene, optionally substituted heteroalkynylene, optionally substituted arylene, optionally substituted heteroarylene, optionally substituted carbocyclylene, optionally substituted heterocyclylene, optionally substituted acylene, optionally substituted heteroatoms, or any combination thereof.

In certain embodiments, the compound of Formula (A) is of Formula (A-1):

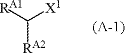

or a salt thereof; the compound of Formula (B) is of Formula (B-1):

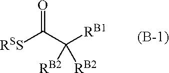

or a salt thereof; and the compound of Formula (C) is of Formula (C-1):

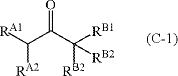

or a salt thereof, wherein:
X¹ is halogen or a leaving group;
R^{S} is optionally substituted alkyl, optionally substituted carbocyclyl, optionally substituted aryl, optionally substituted heterocyclyl, or optionally substituted heteroaryl;
each instance of R^{A1}, R^{A2}, R^{B1}, and R^{B2} is independently hydrogen, optionally substituted alkyl, optionally substituted alkenyl, optionally substituted alkynyl, optionally substituted aryl, optionally substituted carbocyclyl, optionally substituted heteroaryl, or optionally substituted heterocyclyl; optionally wherein R^{A1} and R^{B1} are joined together via a linker.

In certain embodiments, R^{A1} is a small molecule or part of a small molecule. In certain embodiments, R^{B1} and R^{B2} are independently small molecules or parts of small molecules. Small molecules encompass complex small molecules, such as natural products, pharmaceutical agents, and fragments thereof, and intermediates thereto.

The Ni/Zr-mediated coupling reactions provided herein may be performed in an intramolecular fashion to yield cyclic ketones as shown in Scheme 1C.

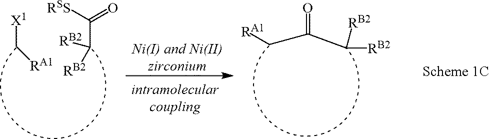

As shown in Scheme 1C, provided herein are methods for preparing a compound of Formula (C-2):

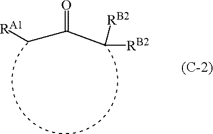

or salt thereof, comprising reacting a compound of Formula (A-B):

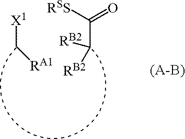

or a salt thereof, in the presence of a nickel(I) complex, a nickel(II) complex, and a zirconium complex; wherein:
R^{A1} and R^{B2} are optionally substituted alkyl, optionally substituted alkenyl, optionally substituted alkynyl, optionally substituted aryl, optionally substituted carbocyclyl, optionally substituted heteroaryl, or optionally substituted heterocyclyl;
X¹ is halogen or a leaving group;
R^{S} is optionally substituted alkyl, optionally substituted carbocyclyl, optionally substituted aryl, optionally substituted heterocyclyl, or optionally substituted heteroaryl; and

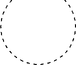

represents a linker.

As described herein, a feature of the present disclosure is the use of a nickel(I) catalyst in conjunction with a nickel(II) catalyst. Without wishing to be bound by any particular theory, the nickel(I) catalyst selectively activates the compound of Formula (A) and the nickel(II) catalyst selectively activates the compound of Formula (B). In certain embodiments, this dual catalyst system leads to improved coupling efficiency and eliminates the need for an excess of one of the coupling partners (i.e., a compound of Formula (A) or (B)). This improvement can be important for reactions involving coupling partners that are structurally complex, expensive, and/or difficult to access (e.g., in the synthesis of halichondrins and analogs thereof).

In certain embodiments, in a Ni/Zr-mediated coupling described herein, the compound of Formula (A) is present in a range from about 1 equivalent to about 1.3 equivalents with respect to the compound of Formula (B). In certain embodiments, the compound of Formula (A) is present in about 1, 1.05, 1.1, 1.15, 1.2, 1.25, or 1.3 equivalents with respect to the compound of Formula (B). In certain embodiments, the compound of Formula (A) and the compound of Formula (B) are present in approximately 1:1 molar ratio.

In certain embodiments, the compound of Formula (C) is isolated in 80% yield or greater when any of the aforementioned ratios of coupling partners is used. In certain embodiments, the compound of Formula (C) is isolated in approximately 85% yield or greater. In certain embodiments, the compound of Formula (C) is isolated in approximately 90% yield or greater. In certain embodiments, the compound of Formula (C) is isolated in approximately 95% yield or greater. In certain embodiments, the compound of Formula (C) is isolated in approximately 98% yield or greater.

As described herein, the Ni/Zr-mediated coupling reactions are carried out in the presence of a nickel(I) complex, a nickel(II) complex, and a zirconium complex. The nickel(I) and nickel(II) complexes (e.g., nickel salt, nickel complex, nickel catalyst, or nickel pre-catalyst) may be any known or available complexes in the art.

In certain embodiments, the nickel(I) complex is of the formula: NiX•(ligand); wherein X is halogen. In certain embodiments, “ligand” is a tridentate ligand. In certain embodiments, the ligand is a tripyridyl ligand. In certain embodiments, the nickel(I) complex is of the formula:

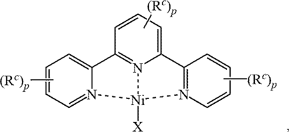

wherein:
X is a halogen;
each instance of p is independently 0 or an integer from 1-4, inclusive;
each instance of R^{c} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, —OR^{O}, or —SR^{S1};
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or two R^{N} bonded to the same nitrogen atom are taken together with the intervening atoms to form optionally substituted heterocyclyl or optionally substituted heteroaryl;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{S1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

In certain embodiments, the nickel(I) complex is of the formula:

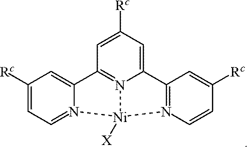

For example, in certain embodiments, the nickel(I) complex is of the formula:

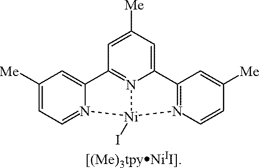

In certain embodiments, the nickel(I) complex is of one of the following formulae:

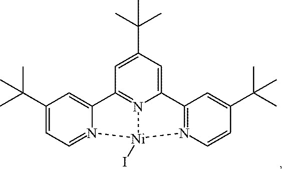

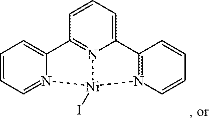

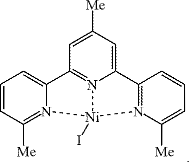

In certain embodiments, the nickel(I) complex is used after being formed by complexation of a nickel source and the “ligand” in solution. In certain embodiments, the nickel source is of the formula: NiX₂; wherein X is halogen. In certain embodiments, the nickel source is NiBr₂, NiI₂, or NiCl₂. In certain embodiments, the nickel source is NiI₂. In certain embodiments, the “ligand” is of the following formula:

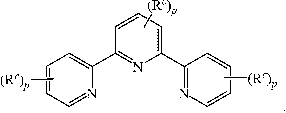

or a salt thereof.

In certain embodiments, the “ligand” is of the formula:

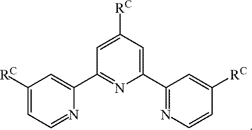

For example, in certain embodiments, the “ligand” is of the formula:

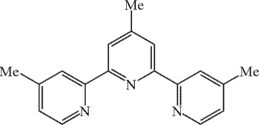 [SMILES: Cc1ccnc(-c2cc(C)cc(-c3cc(C)ccn3)n2)c1]

[(Me)₃tpy], or a salt thereof.

In certain embodiments, the “ligand” is of one of the following formulae:

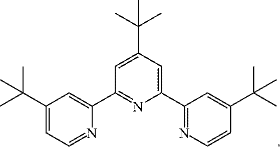

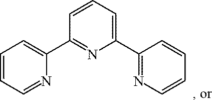

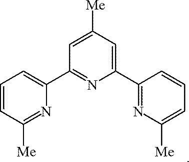

In certain embodiments, the “ligand” is one of the following tridentate ligands:

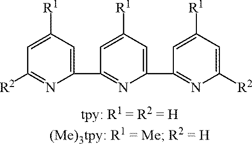

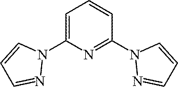 [SMILES: c1cc(-n2cccn2)nc(-n2cccn2)c1]

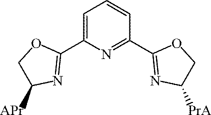

In certain embodiments, the ligand is a bidentate ligand. In certain embodiments, the “ligand” is one of the following bidentate ligands:

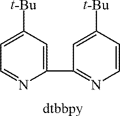

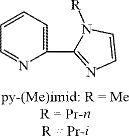

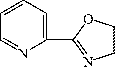 [SMILES: c1ccc(C2=NCCO2)nc1]

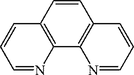 [SMILES: c1cnc2c(c1)ccc1cccnc12]

In certain embodiments, the “ligand” is of the formula:

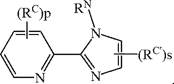

. For example, in certain embodiments, the “ligand” is of the formula:

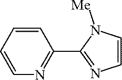 [SMILES: Cn1ccnc1-c1ccccn1]

[(py-(Me)imid)].

In certain embodiments, the nickel(I) complex is present in a catalytic amount. In certain embodiments, the nickel(I) complex is present at approximately 0.001-0.1 mol%, 0.1-1 mol%, 1-5 mol%, 5-10 mol%, 1-10 mol%, 5-20 mol%, 10-20 mol%, 20-30 mol%, 20-40 mol%, 30-40 mol%, 40-50 mol%, 50-60 mol%, 60-70 mol%, 70-80 mol%, or 80-90 mol% with respect to a compound of Formula (A) and/or (B) in the reaction mixture. In certain embodiments, the nickel(I) complex is present in from about 0.1-10 mol% with respect to the compound of Formula (A) and/or the compound of Formula (B). In certain embodiments, the nickel(I) complex is present in about 1 mol% with respect to the compound of Formula (A) and/or the compound of Formula (B). In certain embodiments, the nickel(I) complex is present in from about 1-30 mol% the compound of Formula (A) and/or the compound of Formula (B). In certain embodiments, the nickel(I) complex is present in about 20 mol% with respect to the compound of Formula (A) and/or the compound of Formula (B). In certain embodiments, the nickel(I) complex is present in a stoichiometric or excess amount relative to a compound of Formula (A) and/or (B) in the reaction mixture. In certain embodiments, approximately 1 equivalent of nickel(I) complex is present (i.e., stoichiometric). In other embodiments, greater than 1 equivalent of nickel(I) complex is present (i.e., excess).

In certain embodiments, the nickel(I) complex is present in a catalytic amount. In certain embodiments, the nickel(I) complex is present at approximately 0.001-0.1 mol%, 0.1-1 mol%, 1-5 mol%, 5-10 mol%, 1-10 mol%, 5-20 mol%, 10-20 mol%, 20-30 mol%, 20-40 mol%, 30-40 mol%, 40-50 mol%, 50-60 mol%, 60-70 mol%, 70-80 mol%, or 80-90 mol% with respect to a compound of Formula (A) in the reaction mixture. In certain embodiments, the nickel(I) complex is present in from about 0.1-10 mol% with respect to the compound of Formula (A). In certain embodiments, the nickel(I) complex is present in about 1 mol% with respect to the compound of Formula (A). In certain embodiments, the nickel(I) complex is present in from about 1-30 mol% the compound of Formula (A). In certain embodiments, the nickel(I) complex is present in about 20 mol% with respect to the compound of Formula (A). In certain embodiments, the nickel(I) complex is present in a stoichiometric or excess amount relative to a compound of Formula (A) in the reaction mixture. In certain embodiments, approximately 1 equivalent of nickel(I) complex is present (i.e., stoichiometric). In other embodiments, greater than 1 equivalent of nickel(I) complex is present (i.e., excess).

In certain embodiments, the nickel(II) complex is of the formula: NiX₂•(ligand); wherein X is halogen. In certain embodiments, “ligand” is a bidentate ligand. In certain embodiments, the nickel(II) complex is of the formula:

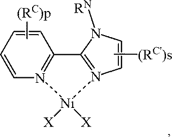

wherein:
each instance of X is a halogen;
p is 0 or an integer from 1-4, inclusive;
s is 0, 1, or 2;
each instance of R^{c} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1};
each instance of R^{c′} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1};
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or two R^{N} bonded to the same nitrogen atom are taken together with the intervening atoms to form optionally substituted heterocyclyl or optionally substituted heteroaryl;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{S1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

For example, in certain embodiments, the nickel(II) complex is of the formula:

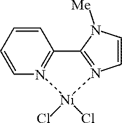

[(py-(Me)imid)•Ni^{II}Cl₂].

In certain embodiments, the nickel(II) complex is of one of the following formulae:

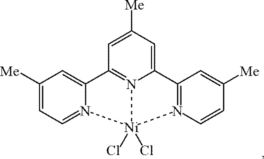

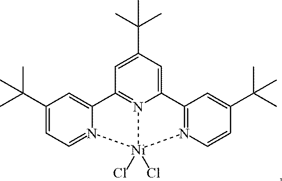

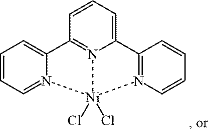

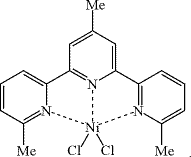

In certain embodiments, the nickel(II) complex is of one of the following formulae:

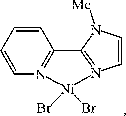

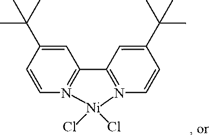

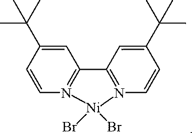

In certain embodiments, the nickel(II) complex is used after being formed by complexation of a nickel source and the “ligand” in solution. In certain embodiments, the nickel source is of the formula: NiX₂; wherein X is halogen. In certain embodiments, the nickel source is NiBr₂, NiI₂, or NiCl₃. In certain embodiments, the nickel source is NiCl₂. In certain embodiments, the “ligand” is of the formula:

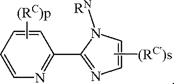

. For example, in certain embodiments, the “ligand” is of the formula:

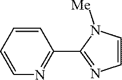 [SMILES: Cn1ccnc1-c1ccccn1]

[(py-(Me)imid)].

In certain embodiments, the “ligand” is a tridentate ligand. In certain embodiments, the “ligand” is a tripyridyl ligand. In certain embodiments, the “ligand” is of the following formula:

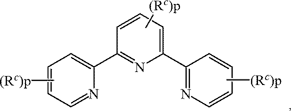

or a salt thereof.

In certain embodiments, the “ligand” is of the formula:

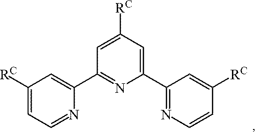

or a salt thereof.

For example, in certain embodiments, the “ligand” is of the formula:

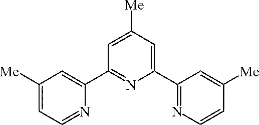 [SMILES: Cc1ccnc(-c2cc(C)cc(-c3cc(C)ccn3)n2)c1]

[(Me)₃tpy], or a salt thereof.

In certain embodiments, the “ligand” is one of the following tridentate ligands:

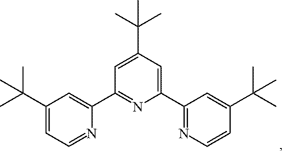

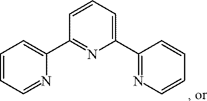

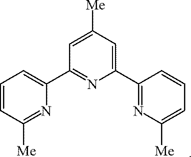

In certain embodiments, the “ligand” is one of the following tridentate ligands:

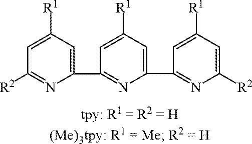

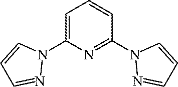 [SMILES: c1cc(-n2cccn2)nc(-n2cccn2)c1]

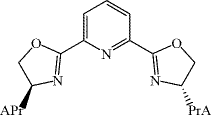

In certain embodiments, the “ligand” is one of the following bidentate ligands:

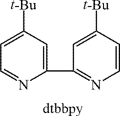

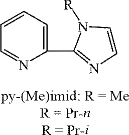

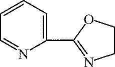 [SMILES: c1ccc(C2=NCCO2)nc1]

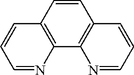 [SMILES: c1cnc2c(c1)ccc1cccnc12]

In certain embodiments, the nickel(II) complex is present in a catalytic amount. In certain embodiments, the nickel(II) complex is present at approximately 0.001-0.1 mol%, 0.1-1 mol%, 1-5 mol%, 5-10 mol%, 1-10 mol%, 5-20 mol%, 10-20 mol%, 20-30 mol%, 20-40 mol%, 30-40 mol%, 40-50 mol%, 50-60 mol%, 60-70 mol%, 70-80 mol%, or 80-90 mol% with respect to a compound of Formula (A) and/or (B) in the reaction mixture. In certain embodiments, the nickel(II) complex is present at from about 0.1-10 mol% with respect to the compound of Formula (A) and/or the compound of Formula (B). In certain embodiments, the nickel(II) complex is present at about 1 mol% with respect to the compound of Formula (A) and/or the compound of Formula (B). In certain embodiments, the nickel(II) complex is present at from about 1-20 mol% with respect to the compound of Formula (A) and/or the compound of Formula (B). In certain embodiments, the nickel(II) complex is present at about 5 mol% with respect to the compound of Formula (A) and/or the compound of Formula (B). In certain embodiments, the nickel(II) complex is present in a stoichiometric or excess amount relative to a compound of Formula (A) and/or (B) in the reaction mixture. In certain embodiments, approximately 1 equivalent of nickel(II) complex is present (i.e., stoichiometric). In other embodiments, greater than 1 equivalent of nickel(II) complex is present (i.e., excess).

In certain embodiments, the nickel(II) complex is present in a catalytic amount. In certain embodiments, the nickel(II) complex is present at approximately 0.001-0.1 mol%, 0.1-1 mol%, 1-5 mol%, 5-10 mol%, 1-10 mol%, 5-20 mol%, 10-20 mol%, 20-30 mol%, 20-40 mol%, 30-40 mol%, 40-50 mol%, 50-60 mol%, 60-70 mol%, 70-80 mol%, or 80-90 mol% with respect to a compound of Formula (B) in the reaction mixture. In certain embodiments, the nickel(II) complex is present at from about 0.1-10 mol% with respect to the compound of Formula (B). In certain embodiments, the nickel(II) complex is present at about 1 mol% with respect to the compound of Formula (B). In certain embodiments, the nickel(II) complex is present at from about 1-20 mol% with respect to the compound of Formula (B). In certain embodiments, the nickel(II) complex is present at about 5 mol% with respect to the compound of Formula (B). In certain embodiments, the nickel(II) complex is present in a stoichiometric or excess amount relative to a compound of Formula (B) in the reaction mixture. In certain embodiments, approximately 1 equivalent of nickel(II) complex is present (i.e., stoichiometric). In other embodiments, greater than 1 equivalent of nickel(II) complex is present (i.e., excess).

As described above, the Ni/Zr-mediated coupling reactions are carried out in the presence of a zirconium complex. In certain embodiments, the zirconium complex is a zirconium(IV) complex. In certain embodiments, the zirconium complex is of the formula (ligand)ₙZrX₂; wherein n is the number of ligands (e.g., 0, 1, 2, 3, 4); and X is halogen (e.g., Cl, Br, I, or F). In certain embodiments, n is 2; and each ligand is independently optionally substituted cyclopentadienyl. In certain embodiments, n is 2; and each ligand is cyclopentadienyl. In certain embodiments, each X is chlorine.

In certain embodiments, the zirconium complex is Cp₂ZrX₂. In certain embodiments, the zirconium complex is Cp₂ZrCl₂.

In certain embodiments, the zirconium complex is Bis(cyclopentadienyl)zirconium(IV) dichloride (Cp₂ZrCl₂), Bis(cyclopentadienyl)dimethylzirconium(IV), Bis(cyclopentadienyl)zirconium(IV) chloride hydride, Bis(butylcyclopentadienyl)zirconium(IV) dichloride, Dimethylbis(pentamethylcyclopentadienyl)zirconium(IV), Bis(methylcyclopentadienyl)zirconium(IV) dichloride, Dichloro[rac-ethylenebis(indenyl)]zirconium(IV), Bis(cyclopentadienyl)zirconium(IV) dihydride, or Dichloro[rac-ethylenebis(4,5,6,7-tetrahydro-1-indenyl)]zirconium(IV).

In certain embodiments, the zirconium complex is present in a catalytic amount. In certain embodiments, the zirconium complex is present in between 0.001-0.1 mol%, 0.1-1 mol%, 1-5 mol%, 5-10 mol%, 1-10 mol%, 5-20 mol%, 10-20 mol%, 20-30 mol%, 30-40 mol%, 40-50 mol%, 50-60 mol%, 60-70 mol%, 70-80 mol%, or 80-90 mol% with respect to a compound of Formula (A) or (B) in the reaction mixture. In certain embodiments, the zirconium complex is present in a stoichiometric or excess amount relative to a compound of Formula (A) or (B) in the reaction mixture. In certain embodiments, approximately 1 equivalent of zirconium complex is present (i.e., stoichiometric). In other embodiments, greater than 1 equivalent of zirconium complex is present (i.e., excess). In certain embodiments, the zirconium complex is present in about 1 to about 5 equivalents with respect to the compound of Formula (A) or the compound of Formula (B). In certain embodiments, the zirconium complex is present in about 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 equivalents with respect to the compound of Formula (A) or the compound of Formula (B). In certain embodiments, the zirconium complex is present in about 1 equivalent with respect to the compound of Formula (A) or the compound of Formula (B).

In certain embodiments, a Ni/Zr-mediated coupling reaction provided herein is performed in the presence of one or more additional reagents or catalysts, such as a reducing metal. Any reducing metal can be used in the coupling described herein. In certain embodiments, the reducing metal is zinc or manganese. The zinc or manganese may be present in a catalytic, stoichiometric, or excess amount. In certain embodiments, the zinc or manganese is present in excess (i.e., greater than 1 equivalent) with respect to a compound of Formula (A) or Formula (B). In certain embodiments, between 1 and 10 equivalents of zinc or manganese is used. In certain embodiments, approximately 1.0, 1.5, 2, 2.5, 3, 3.5, 4, 4.5, 5, 5.5, 6, 6.5, 7, 7.5, 8, 8.5, 9, 9.5 or 10 equivalents of zinc or manganese is present. In certain embodiments, approximately 6 equivalents of zinc or manganese is used. In certain embodiments, approximately 3 equivalents of zinc or manganese is used.

In certain embodiments, the reducing metal is zinc. In certain embodiments, the reducing metal is manganese. In certain embodiments, zinc metal is used (i.e., zinc(0)). In certain embodiments, manganese metal is used (i.e., manganese(0)). In certain embodiments, the reaction is carried out in the presence of zinc powder, zinc foil, zinc beads, or any other form of zinc metal. In certain embodiments, a zinc salt is employed such as zinc acetate, zinc sulfate, zinc chloride, zinc bromide, zinc iodide, zinc fluoride, zinc sulfide, or zinc phosphate.

In certain embodiments, the coupling reaction is carried out in the presence of one or more reagents which help activate zinc metal in the reaction (e.g., by clearing the surface of zinc oxide). In certain embodiments, the reaction is carried out in the presence of a trialkylsilyl halide (e.g., triethylsilyl chloride (TESCl)). This reagent may be present in a catalytic, stoichiometric, or excess amount with respect to a compound of Formula (A) or Formula (B). In certain embodiments, approximately 1.5, 2, 2.5, 3, 3.5, 4, 4.5, 5, 5.5, 6, 7, 8, 9, or 10 equivalents of this reagent is present with respect to a compound of Formula (A) or Formula (B). In certain embodiments, approximately 1.5 equivalents of this reagent is present with respect to a compound of Formula (A) or Formula (B).

In certain embodiments, the Ni/Zr-mediated coupling is carried out in the presence of one or more additional reagents (i.e., in addition to nickel, zirconium, and zinc; or in addition to nickel, zirconium, and manganese).

In certain embodiments, the Ni/Zr-mediated coupling reaction is carried out in the presence of a base or proton scavenger. In certain embodiments, the base is a pyridine base. In certain embodiments, the base is 2,6-di-tert-butyl pyridine. In certain embodiments, the base is 2,6-lutidine. In certain embodiments, the base is 2,6-di-tert-butyl-4-methylpyridine. In certain embodiments, the base is used in a stoichiometric or excess amount with respect to a compound of Formula (A) or Formula (B). In certain embodiments, approximately 1 equivalent to 10 equivalents of the base or proton scavenger is employed with respect to a compound of Formula (A) or Formula (B). In certain embodiments, approximately 1.0, 1.5, 2, 2.5, 3, 3.5, 4, 4.5, 5, 5.5, 6, 7, 8, 9, or 10 equivalents of the base or proton scavenger is present with respect to a compound of Formula (A) or Formula (B). In certain embodiments, approximately 2.5 equivalents of the base or proton scavenger is employed with respect to a compound of Formula (A) or Formula (B).

In certain embodiments, the Ni/Zr-mediated coupling described herein is carried out in a solvent. Any solvent may be used, and the scope of the method is not limited to any particular solvent or mixture of solvents. The solvent may be polar or non-polar, protic or aprotic, or a combination of solvents (e.g., co-solvents). Examples of useful organic solvents are provided herein. In certain embodiments, the solvent comprises N,N-dimethylacetamide (DMA). In certain embodiments, the solvent comprises 1,2-dimethoxyethane (DME). In certain embodiments, the solvent is a DMA/DME mixture (e.g., 1:1).

In certain embodiments, the solvent comprises 1,3-dimethyl-2-imidazolidinone (DMI). In certain embodiments, the coupling reaction is carried out in a DMI/tetrahydrofuran (THF) mixture. In certain embodiments, the coupling reaction is carried out in a DMI/ethyl acetate (EtOAc) mixture.

In certain embodiments, the coupling reaction is carried out in a DMI/DME mixture (e.g., 1:1). In certain embodiments, the coupling reaction is carried out in approximately 1:1 DMI/DME.

The Ni/Zr-mediated coupling reactions described herein may be carried out at any concentration in solvent. Concentration refers to the molar concentration (mol/L) of a coupling partners (e.g., compounds of Formula (A) or (B)) in a solvent. In certain embodiments, the concentration is approximately 0.05, 0.1, 0.2, 0.3, 0.4, 0.5, 0.6, 0.7, 0.8, or 0.9 M. In certain embodiments, the concentration is about 0.2 M. In certain embodiments, the concentration is approximately 0.5 M. In certain embodiments, the concentration is greater than 1 M. In certain embodiments, the concentration is less than 0.1 M.

The Ni/Zr-mediated coupling reactions described herein can be carried out at any temperature. In certain embodiments, the reaction is carried out at around room temperature (i.e., between 18 and 24° C.). In certain embodiments, the reaction is carried out below room temperature (e.g., between 0° C. and room temperature). In certain embodiments, the reaction is carried out at above room temperature (e.g., between room temperature and 100° C.). In certain embodiments, the reaction is carried out at a temperature ranging from approximately room temperature to approximately 100° C. In certain embodiments, the reaction is carried out at a temperature ranging from approximately room temperature to approximately 50° C.

In certain embodiments, the reaction is carried out in the presence of a nickel(I) complex, a nickel(II) complex, a zirconium complex, and a reducing metal.

In certain embodiments, the reaction is carried out in the presence of a nickel (I) complex of the formula: NiX•(ligand); a nickel (II) complex of the formula: NiX₂•(ligand); a zirconium complex of the formula: (ligand)ₙZrX₂; and zinc or manganese metal.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; and zinc or manganese metal.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; and zinc metal.

In certain embodiments, the reaction is carried out in the presence of approximately 1 mol% the nickel (I) complex: (Me)₃tpy•Ni^{I}I; approximately 1 mol% of the nickel(II) complex: (py-(Me)imid)•Ni^{II}Cl₂; approximately 1 equivalent of the zirconium complex: Cp₂ZrCl₂; and approximately 3 equivalents of zinc metal. In certain embodiments, the reaction is carried out in a mixture of DMA/DME. In certain embodiments, the reaction is carried out at around room temperature.

In certain embodiments, the reaction is carried out in the presence of approximately 1 mol% the nickel (I) complex: (Me)₃tpy•Ni^{I}I; approximately 1 mol% of the nickel(II) complex: (py-(Me)imid)•Ni^{II}Cl₂; approximately 1 equivalent of the zirconium complex: Cp₂ZrCl₂; and approximately 3 equivalents of zinc metal, in a mixture of DMA/DME (e.g., 1:1 DMA/DME; 0.5 M) at around room temperature.

In certain embodiments, the reaction is carried out in the presence of a nickel(I) complex, a nickel(II) complex, a zirconium complex, a reducing metal, and a base or proton scavenger.

In certain embodiments, the reaction is carried out in the presence of a nickel (I) complex of the formula: NiX•(ligand); a nickel (II) complex of the formula: NiX₂•(ligand); a zirconium complex of the formula: (ligand)ₙZrX₂; zinc or manganese metal; and a base or proton scavenger.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; zinc or manganese metal; and a base or proton scavenger.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; zinc metal; and 2,6-di-tert-butyl-4-methylpyridine.

In certain embodiments, the reaction is carried out in the presence of approximately 20 mol% the nickel (I) complex: (Me)₃tpy•Ni^{I}I; approximately 5 mol% of the nickel(II) complex: (py-(Me)imid)•Ni^{II}Cl₂; approximately 1 equivalent of the zirconium complex: Cp₂ZrCl₂; approximately 6 equivalents of zinc metal; and approximately 2.5 equivalents of 2,6-di-tert-butyl-4-methylpyridine. In certain embodiments, the reaction is carried out in a mixture of DMA/DME. In certain embodiments, the reaction is carried out at around room temperature.

In certain embodiments, the reaction is carried out in the presence of approximately 20 mol% the nickel (I) complex: (Me)₃tpy•Ni^{I}I; approximately 5 mol% of the nickel(II) complex: (py-(Me)imid)•Ni^{II}Cl₂; approximately 1 equivalent of the zirconium complex: Cp₂ZrCl₂; approximately 6 equivalents of zinc metal; and approximately 2.5 equivalents of 2,6-di-tert-butyl-4-methylpyridine, in a mixture of DMA/DME (e.g., 1:1 DMA/DME; 0.2 M) at around room temperature.

### Synthesis of Halichondrins and Analogs

The Ni/Zr-mediated coupling reactions provided herein can be applied to the synthesis of halichondrins (e.g., halichondrin A, B, C; homohalichondrin A, B, C, norhalichondrin A, B, C) and analogs thereof. In certain embodiments, methods are useful in the synthesis of compounds of Formula (H3-A), such as Compound (1). In certain embodiments, the methods comprise the steps of: (1) coupling a “left half” building block with a “right half” building block via a Ni/Zr-mediated coupling reaction provided herein; optionally followed by (2) cyclizing the resulting coupling product (e.g., acid-mediated cyclization); and optionally followed by any necessary synthetic transformations to arrive at a desired product.

### Synthesis of Halichondrins

The Ni/Zr-mediated coupling reactions provided herein can be applied to the preparation of halichondrins (e.g., halichondrin A, B, C) and analogs thereof. For example, as shown in Scheme 2A, coupling of a left half of Formula (L-2-14) with a right half of Formula (R-2-I) via a Ni/Zr-mediated coupling yields a ketone of Formula (H-2-II), cyclization of which provides a compound of Formula (H-2-I), which is a halichondrin or an analog thereof, or an intermediate thereto.

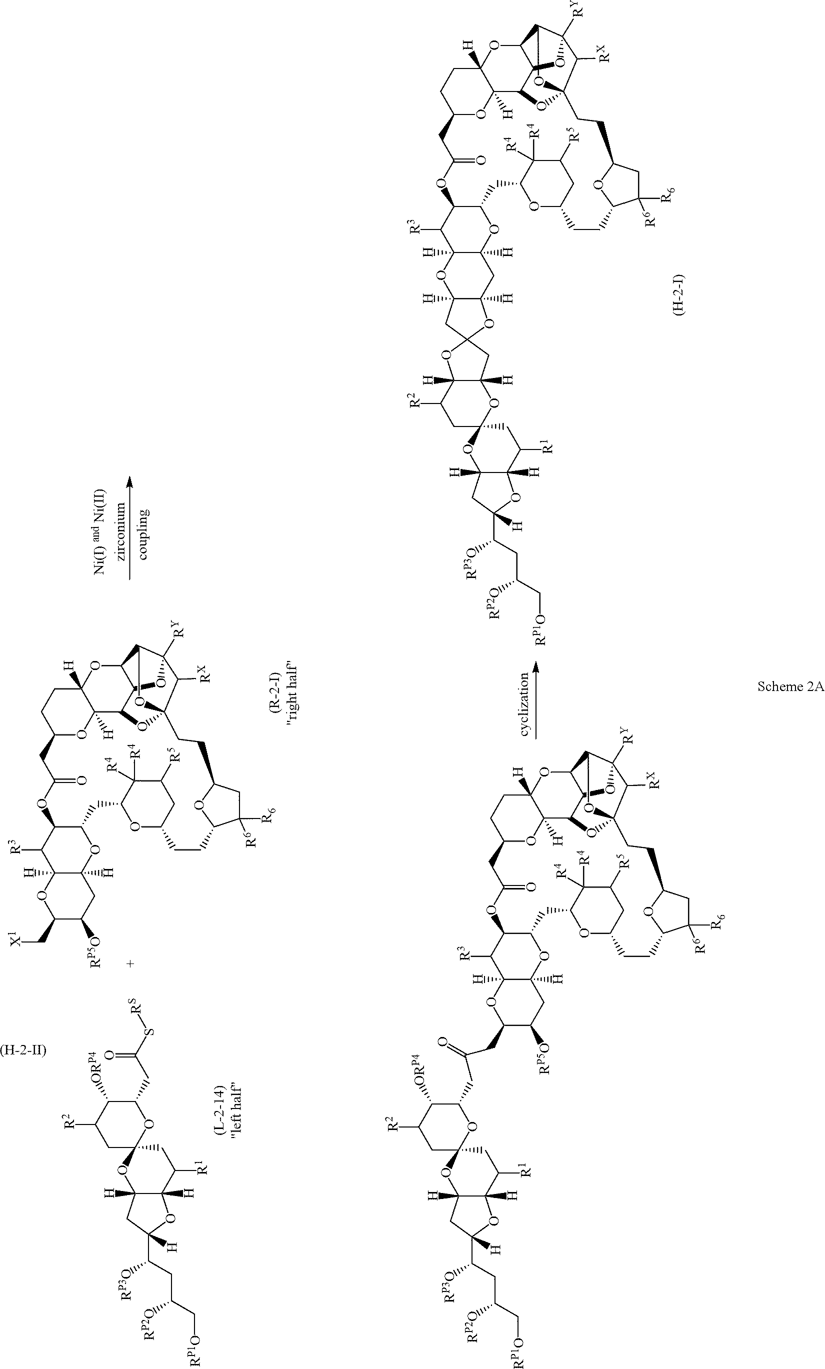

In certain embodiments, the compound of Formula (A) is of Formula (R-2-I); the compound of Formula (B) is of Formula (L-2-14); and the compound of Formula (C) is of the Formula (H-2-II). Accordingly, provided herein is a method of preparing a compound of Formula (H-2-II):

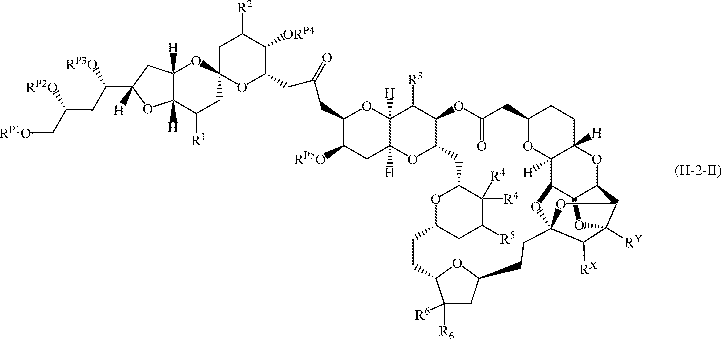

or a salt or stereoisomer thereof, the method comprising coupling a compound of Formula (L-2-14):

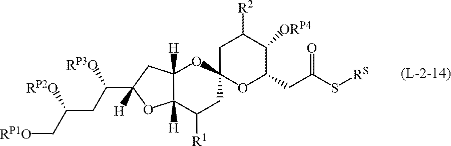

or a salt or stereoisomer thereof, with a compound of Formula (R-2-I):

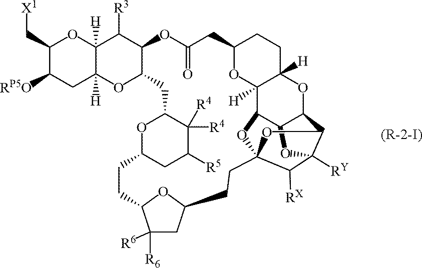

or a salt or stereoisomer thereof, in the presence of a nickel(I) complex, a nickel(II) complex, and a zirconium complex, wherein:
R^{S} is optionally substituted alkyl, optionally substituted carbocyclyl, optionally substituted aryl, optionally substituted heterocyclyl, or optionally substituted heteroaryl;
X¹ is halogen or a leaving group;
R¹, R², R³, and R⁵ are each independently hydrogen, halogen, or optionally substituted alkyl;
each instance of R⁴ is independently hydrogen, halogen, or optionally substituted alkyl, or two R⁴ groups are taken together to form:

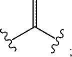

each instance of R⁶ is independently hydrogen, halogen, or optionally substituted alkyl, or two R⁶ groups are taken together to form:

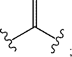

R^{P1}, R^{P2}, R^{P3}, R^{P4}, and R^{P5} are each independently hydrogen, optionally substituted alkyl, optionally substituted acyl, or an oxygen protecting group;
R^{X} is hydrogen or -OR^{Xa}, wherein R^{Xa} is hydrogen, optionally substituted alkyl, optionally substituted acyl, or an oxygen protecting group; and
R^{Y} is hydrogen or -OR^{Ya}, wherein R^{Ya} is hydrogen, optionally substituted alkyl, optionally substituted acyl, or an oxygen protecting group;
optionally wherein R^{Xa} and R^{Ya} are joined together with their intervening atoms to form optionally substituted heterocyclyl.

In certain embodiments, the step of coupling to provide a compound of Formula (H-2-II) is a Ni/Zr-mediated coupling reaction provided herein. Any reagents or conditions provided herein for the Ni/Zr-mediated coupling may be used in the coupling.

Additional methods for converting compounds of Formula (H-2-II) into compounds of Formula (H-2-I) (e.g., halichondrins and analogs thereof) can be found in International Publication No. WO 2019/010363, published Jan. 10, 2019, which is incorporated herein by reference. For example, in certain embodiments, the method described above may further comprise a step of cyclizing a compound of Formula (H-2-II):

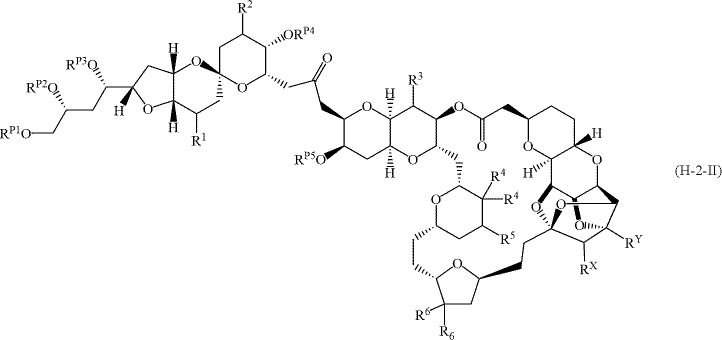

or a salt or stereoisomer thereof, to yield a compound of Formula (H-2-I):

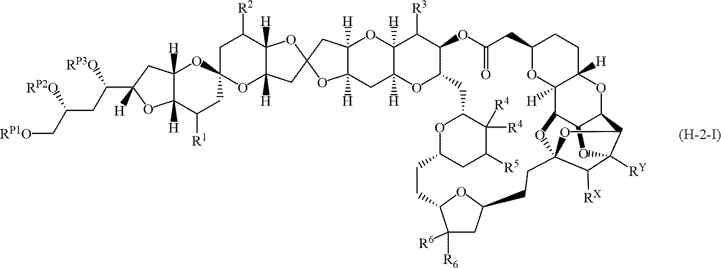

or a salt or stereoisomer thereof.

### Synthesis of Homohalichrondrins

The Ni/Zr-mediated coupling reactions provided herein can be applied to the preparation of homohalichondrins (e.g., homohalichondrin A, B, C), and analogs thereof. For example, as shown in Scheme 2B, coupling of a left half of Formula (L-2-16) with a right half of Formula (R-2-I) via a Ni/Zr-mediated coupling yields a ketone of Formula (HH-2-II), cyclization of which provides a compound of Formula (HH-2-I), which is a homohalichondrin natural product or an analog thereof, or an intermediate thereto.

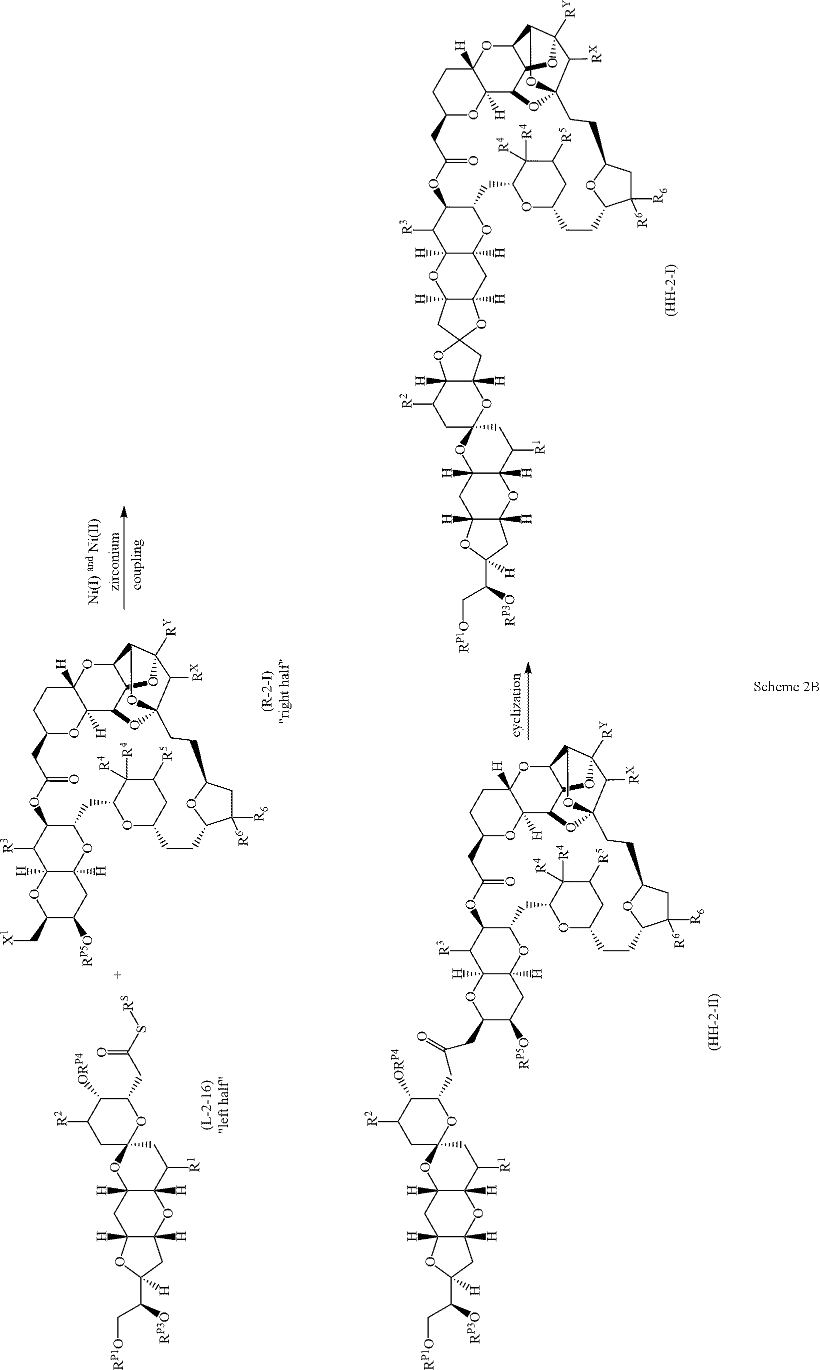

In certain embodiments, the compound of Formula (A) is of Formula (R-2-I); the compound of Formula (B) is of Formula (L-2-16); and the compound of Formula (C) is of the Formula (HH-2-II). Provided herein is a method of preparing a compound of Formula (HH-2-II):

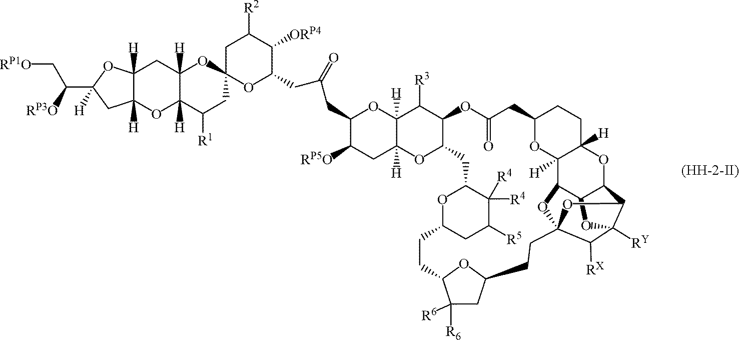

or a salt or stereoisomer thereof, the method comprising coupling a compound of Formula (L-2-16):

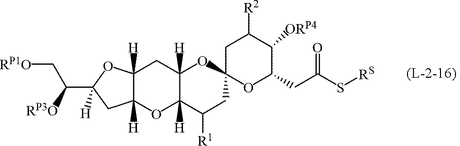

or a salt or stereoisomer thereof, with a compound of Formula (R-2-I):

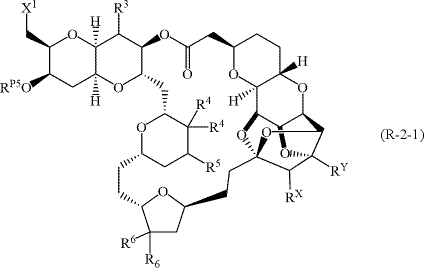

or a salt or stereoisomer thereof, in the presence of a nickel(I) complex, a nickel(II) complex, and a zirconium complex, wherein:
R^{S} is optionally substituted alkyl, optionally substituted carbocyclyl, optionally substituted aryl, optionally substituted heterocyclyl, or optionally substituted heteroaryl;
X¹ is halogen or a leaving group;
R¹, R², R³, and R⁵ are each independently hydrogen, halogen, or optionally substituted alkyl;
each instance of R⁴ is independently hydrogen, halogen, or optionally substituted alkyl, or two R⁴ groups are taken together to form:

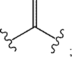

each instance of R⁶ is independently hydrogen, halogen, or optionally substituted alkyl, or two R⁶ groups are taken together to form:

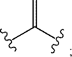

R^{P1}, R^{P3}, R^{P4}, and R^{P5} are each independently hydrogen, optionally substituted alkyl, optionally substituted acyl, or an oxygen protecting group;
R^{x} is hydrogen or -OR^{Xa}, wherein R^{Xa} is hydrogen, optionally substituted alkyl, optionally substituted acyl, or an oxygen protecting group; and
R^{Y} is hydrogen or -OR^{Ya}, wherein R^{Ya} is hydrogen, optionally substituted alkyl, optionally substituted acyl, or an oxygen protecting group;
optionally wherein R^{Xa} and R^{Ya} are joined together with their intervening atoms to form optionally substituted heterocyclyl.

In certain embodiments, the step of coupling to provide a compound of Formula (HH-2-II) is a Ni/Zr-mediated coupling as provided herein. Any reagents or conditions provided herein for the Ni/Zr-mediated coupling may be used in the coupling.

Additional methods for converting compounds of Formula (HH-2-II) into compounds of Formula (HH-2-1) (e.g., homohalichondrins and analogs thereof) can be found in International Publication No. WO 2019/010363, published Jan. 10, 2019, which is incorporated herein by reference. For example, in certain embodiments, the method described above may further comprise a step of cyclizing a compound of Formula (HH-2-II):

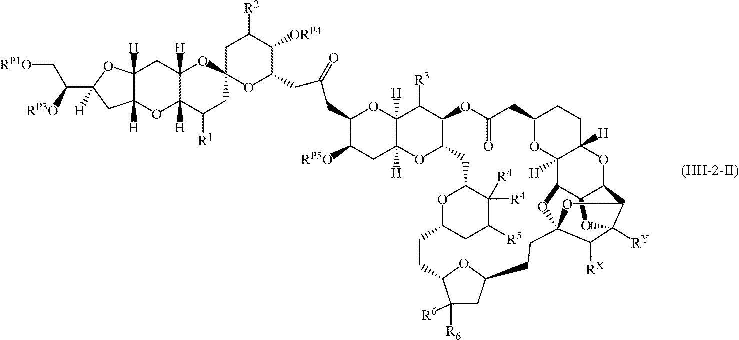

or a salt or stereoisomer thereof, to yield a compound of Formula (HH-2-I):

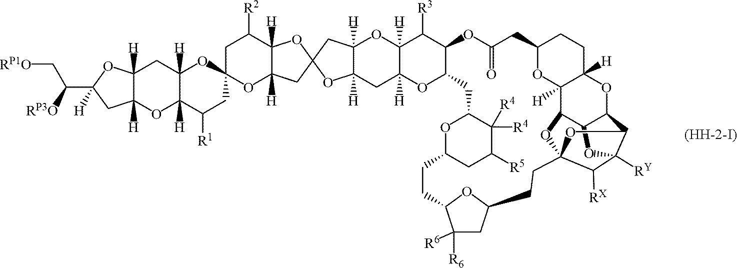

or a salt or stereoisomer thereof.

### Synthesis of Norhalichondrlns

The Ni/Zr-mediated coupling reactions provided herein can be applied to the preparation of norhalichondrins (e.g., norhalichondrin A, B, C) and analogs thereof. For example, as shown in Scheme 2C, coupling of a left half of Formula (L-2-15) with a right half of Formula (R-2-I) via a Ni/Zr-mediated coupling yields a ketone of Formula (NH-2-II), cyclization of which provides a compound of Formula (NH-2-I), which is a norhalichondrin or an analog thereof, or intermediate thereto.

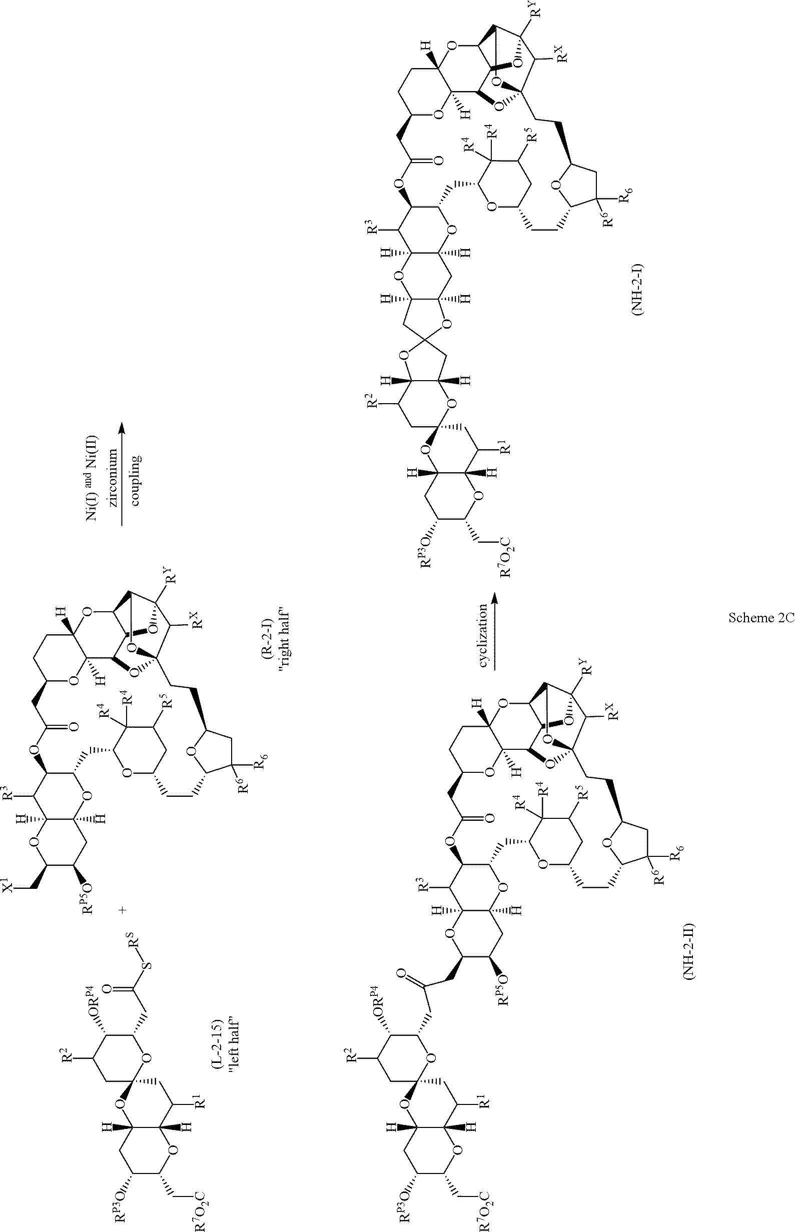

In certain embodiments, the compound of Formula (A) is of Formula (R-2-I); the compound of Formula (B) is of Formula (L-2-15); and the compound of Formula (C) is of the Formula (NH-2-II). Provided herein is a method of preparing a compound of Formula (NH-2-II):

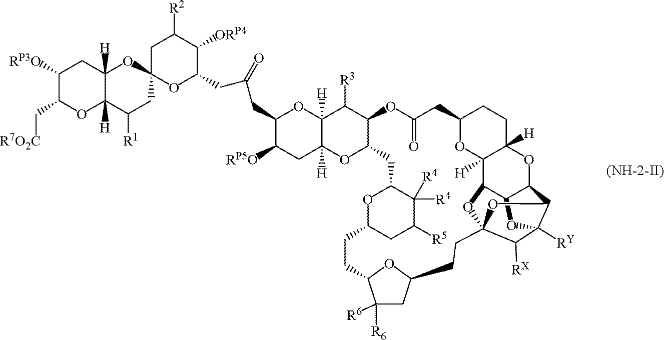

or a salt or stereoisomer thereof, the method comprising coupling a compound of Formula (L-2-15):

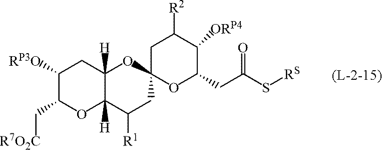

or a salt or stereoisomer thereof, with a compound of Formula (R-2-I):

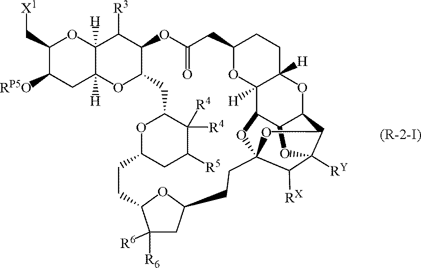

or a salt or stereoisomer thereof, in the presence of a nickel(I) complex, a nickel(II) complex, and a zirconium complex, wherein:
R^{s} is optionally substituted alkyl, optionally substituted carbocyclyl, optionally substituted aryl, optionally substituted heterocyclyl, or optionally substituted heteroaryl;
X¹ is halogen or a leaving group;
R¹, R², R³, and R⁵ are each independently hydrogen, halogen, or optionally substituted alkyl;
each instance of R⁴ is independently hydrogen, halogen, or optionally substituted alkyl, or two R⁴ groups are taken together to form:

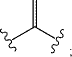

each instance of R⁶ is independently hydrogen, halogen, or optionally substituted alkyl, or two R⁶ groups are taken together to form:

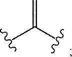

R^{P3} R^{P4}, and R^{P5} are each independently hydrogen, optionally substituted alkyl, optionally substituted acyl, or an oxygen protecting group;
R⁷ is hydrogen, optionally substituted alkyl, optionally substituted carbocyclyl, optionally substituted aryl, optionally substituted heterocyclyl, optionally substituted heteroaryl, optionally substituted acyl, or an oxygen protecting group;
R^{X} is hydrogen or -OR^{Xa}, wherein R^{Xa} is hydrogen, optionally substituted alkyl, optionally substituted acyl, or an oxygen protecting group; and
R^{Y} is hydrogen or -OR^{Ya}, wherein R^{Ya} is hydrogen, optionally substituted alkyl, optionally substituted acyl, or an oxygen protecting group;
optionally wherein R^{Xa} and R^{Ya} are joined together with their intervening atoms to form optionally substituted heterocyclyl.

In certain embodiments, the step of coupling to provide a compound of Formula (NH-2-II) is a Ni/Zr-mediated coupling provided herein. Any reagents or conditions provided herein for the Ni/Zr-mediated coupling may be used in the coupling.

Additional methods for converting compounds of Formula (NH-2-II) into compounds of Formula (NH-2-I) (e.g., norhalichondrins and analogs thereof) can be found in International Publication No. WO 2019/010363, published Jan. 10, 2019, which is incorporated herein by reference. For example, in certain embodiments, the method described above may further comprise a step of cyclizing a compound of Formula (NH-2-II):

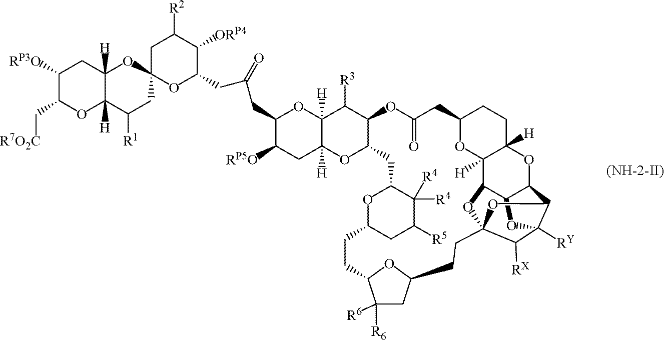

or a salt or stereoisomer thereof, to yield a compound of Formula (NH-2-I):

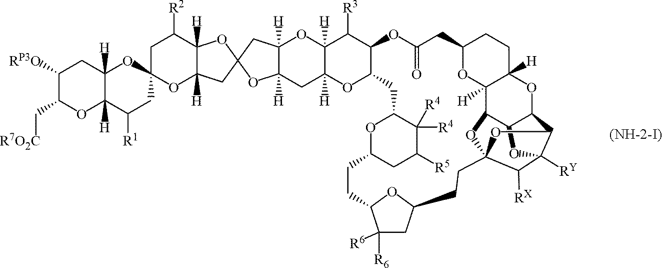

or a salt or stereoisomer thereof.

### Synthesis of Additional Halichondrin Analogs

Methods for the preparation of additional halichondrin analogs are provided herein. The Ni/Zr-mediated coupling reactions provided herein can be applied to the preparation of additional halichondrin analogs. For example, as shown in Scheme 2D, coupling of a left half of Formula (L-2-6) with a right half of Formula (R-2-I) via a Ni/Zr-mediated coupling yields a ketone of Formula (H3-2-II), cyclization of which provides a compound of Formula (H3-2-I). The compound of Formula (H3-2-I) can be subjected to further synthetic transformation to yield a desired compound.

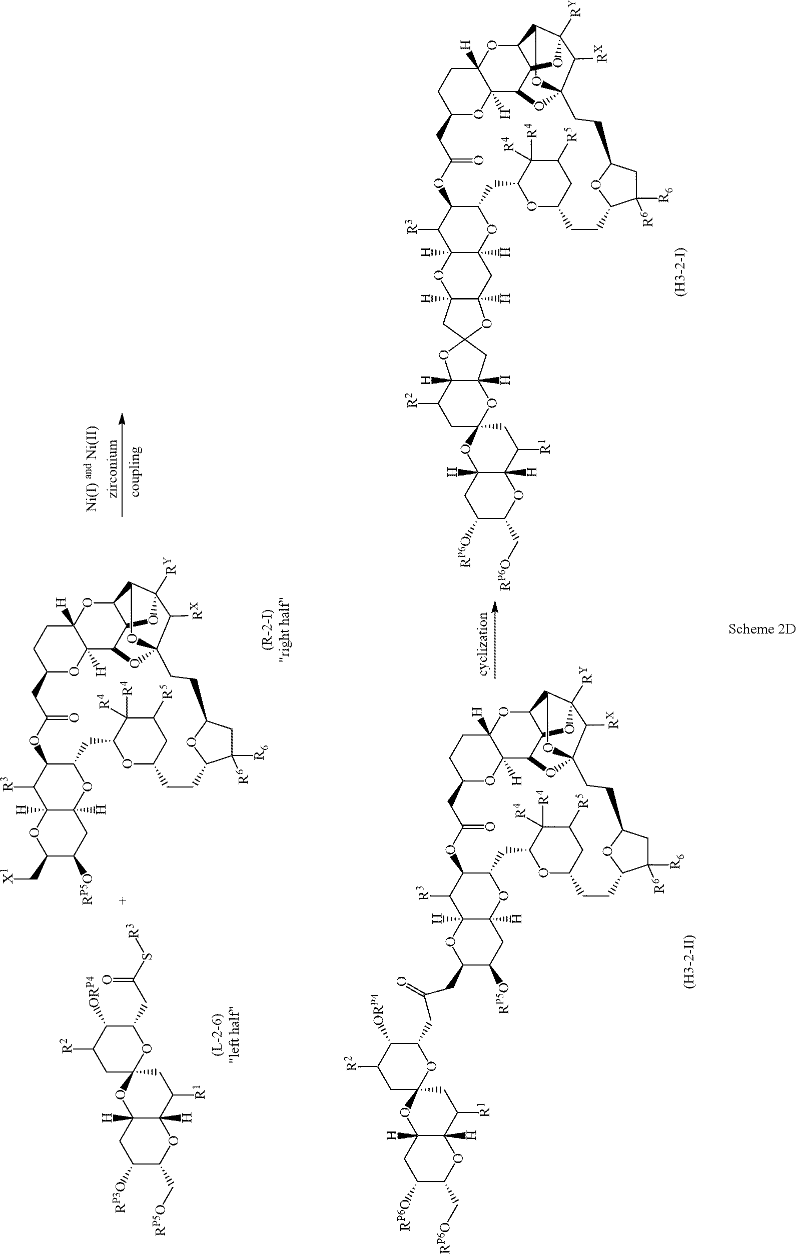

In certain embodiments, the compound of Formula (A) is of Formula (R-2-I); the compound of Formula (B) is of Formula (L-2-6); and the compound of Formula (C) is of the Formula (H3-2-I). Provided herein is a method of preparing a compound of Formula (H3-2-II):

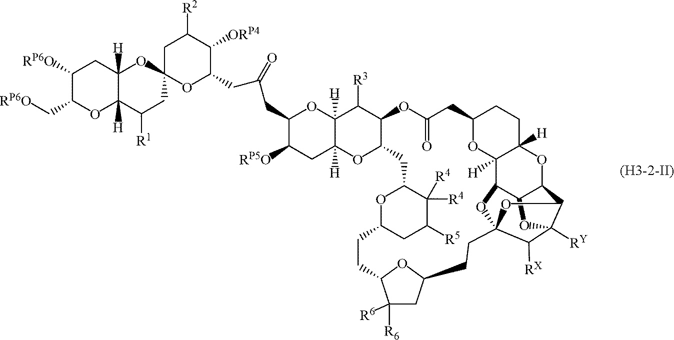

or a salt or stereoisomer thereof, the method comprising coupling a compound of Formula (L-2-6):

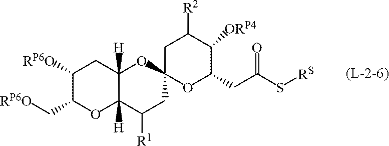

or a salt or stereoisomer thereof, with a compound of Formula (R-2-I):

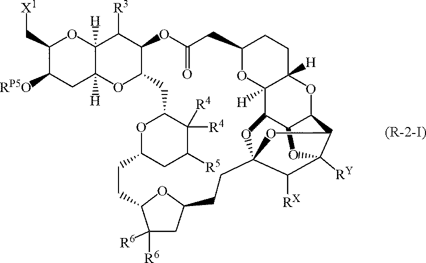

or a salt or stereoisomer thereof, in the presence of a nickel(I) complex, a nickel(II) complex, and a zirconium complex, wherein:
R^{s} is optionally substituted alkyl, optionally substituted carbocyclyl, optionally substituted aryl, optionally substituted heterocyclyl, or optionally substituted heteroaryl;
X¹ is halogen or a leaving group;
R¹, R², R³, and R⁵ are each independently hydrogen, halogen, or optionally substituted alkyl;
each instance of R⁴ is independently hydrogen, halogen, or optionally substituted alkyl, or two R⁴ groups are taken together to form:

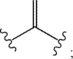

each instance of R⁶ is independently hydrogen, halogen, or optionally substituted alkyl, or two R⁶ groups are taken together to form:

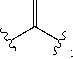

R^{P4}, R^{P5}, and R^{P6} are each independently hydrogen, optionally substituted alkyl, optionally substituted acyl, or an oxygen protecting group; optionally wherein two R^{P6} are joined with the intervening atoms to form optionally substituted heterocyclyl;
R^{X} is hydrogen or -OR^{Xa}, wherein R^{Xa} is hydrogen, optionally substituted alkyl, optionally substituted acyl, or an oxygen protecting group; and
R^{Y} is hydrogen or -OR^{Ya}, wherein R^{Ya} is hydrogen, optionally substituted alkyl, optionally substituted acyl, or an oxygen protecting group;
optionally wherein R^{Xa} and R^{Ya} are joined together with their intervening atoms to form optionally substituted heterocyclyl.

In certain embodiments, the method comprises coupling a compound of Formula (EL):

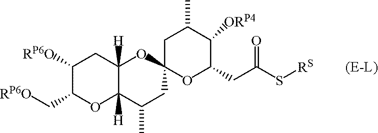

or a salt or stereoisomer thereof, with a compound of the formula (E-R):

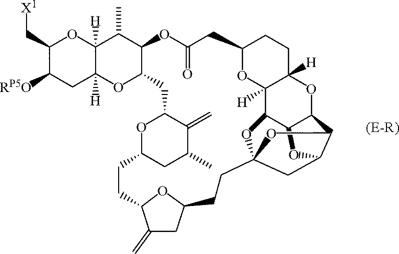

or a salt or stereoisomer thereof, in the presence of a nickel(I) complex, a nickel(II) complex, and a zirconium complex, to yield a compound of the formula (E-1):

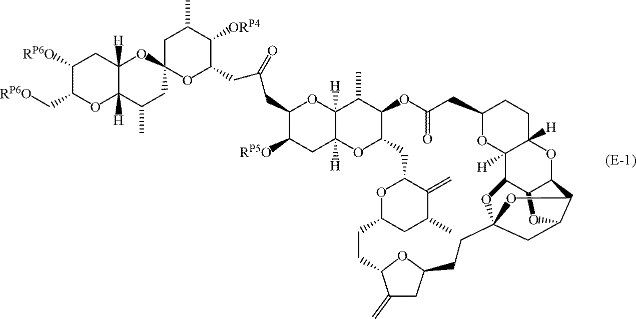

or a salt or stereoisomer thereof.

In certain embodiments, the step of coupling to provide a compound of Formula (H3-2-II), (E-1), or a salt or stereoisomer thereof, is a Ni/Zr-mediated coupling provided herein. Any reagents or conditions provided herein for the Ni/Zr-mediated coupling may be used in the coupling. For example, in certain embodiments, the reaction is carried out in the presence of a nickel(I) complex, a nickel(II) complex, a zirconium complex, a reducing metal, and a base or proton scavenger.

In certain embodiments, the reaction is carried out in the presence of a nickel (I) complex of the formula: NiX•(ligand); a nickel (II) complex of the formula: NiX₂•(ligand); a zirconium complex of the formula: (ligand)ₙZrX₂; zinc or manganese metal; and a base or proton scavenger.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; zinc or manganese metal; and a base or proton scavenger.

In certain embodiments, the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; zinc metal; and 2,6-di-tert-butyl-4-methylpyridine.

In certain embodiments, the reaction is carried out in the presence of approximately 20 mol% the nickel (I) complex: (Me)₃tpy•Ni^{I}I; approximately 5 mol% of the nickel(II) complex: (py-(Me)imid)•Ni^{II}Cl₂; approximately 1 equivalent of the zirconium complex: Cp₂ZrCl₂; approximately 6 equivalents of zinc metal; and approximately 2.5 equivalents of 2,6-di-tert-butyl-4-methylpyridine. In certain embodiments, the reaction is carried out in a mixture of DMA/DME. In certain embodiments, the reaction is carried out at around room temperature.

In certain embodiments, the reaction is carried out in the presence of approximately 20 mol% the nickel (I) complex: (Me)₃tpy•Ni^{I}I; approximately 5 mol% of the nickel(II) complex: (py-(Me)imid)•Ni^{II}Cl₂; approximately 1 equivalent of the zirconium complex: Cp₂ZrC1₂; approximately 6 equivalents of zinc metal; and approximately 2.5 equivalents of 2,6-di-4ert-butyl-4-methylpyridine, in a mixture of DMA/DME (e.g., 1:1 DMA/DME; 0.2 M) at around room temperature.

Additional methods for converting compounds of Formula (H3-2-II) into compounds of Formula (H3-2-I) can be found in International Publication No. WO 2019/010363, published Jan. 10, 2019, which is incorporated herein by reference. For example, in certain embodiments, the method described above may further comprise a step of cyclizing a compound of Formula (H3-2-II):

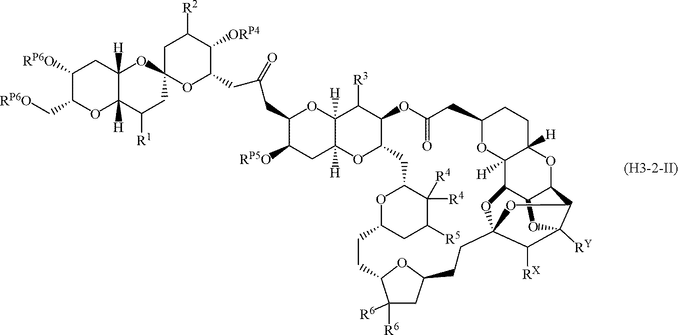

or a salt or stereoisomer thereof, to yield a compound of Formula (H3-2-I):

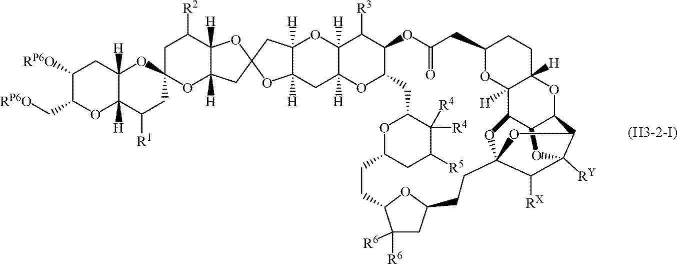

or a salt or stereoisomer thereof

In certain embodiments, the method is a method of preparing Compound (2):

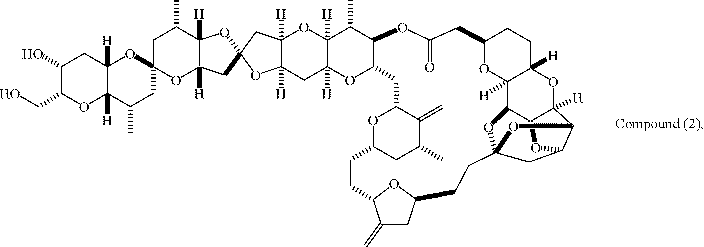 [SMILES: C=C1C[C@@H]2CC[C@@]34C[C@H]5O[C@H]6[C@@H](O3)[C@H]3O[C@H](CC[C@@H]3O[C@H]6[C@H]5O4)CC(=O)O[C@@H]3[C@@H](C)[C@@H]4O[C@@H]5C[C@]6(C[C@@H]7O[C@]8(C[C@H](C)[C@@H]9O[C@H](CO)[C@H](O)C[C@@H]9O8)C[C@H](C)[C@@H]7O6)O[C@@H]5C[C@@H]4O[C@H]3C[C@H]3O[C@@H](CC[C@@H]1O2)C[C@@H](C)C3=C]

or a salt or stereoisomer thereof, the method comprising cyclizing a compound of the formula:

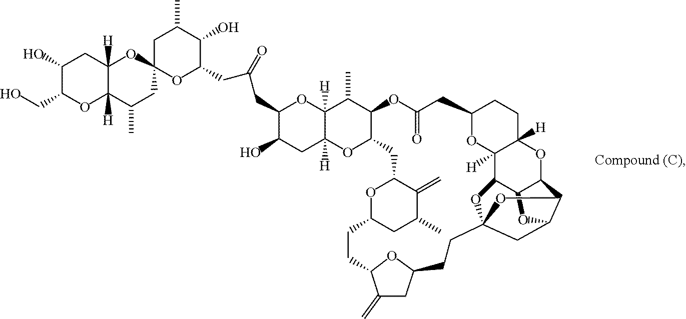 [SMILES: C=C1C[C@@H]2CC[C@@]34C[C@H]5O[C@H]6[C@@H](O3)[C@H]3O[C@H](CC[C@@H]3O[C@H]6C5O4)CC(=O)O[C@@H]3[C@@H](C)[C@@H]4O[C@H](CC(=O)C[C@@H]5O[C@@]6(C[C@H](C)[C@@H]5O)C[C@H](C)[C@@H]5O[C@H](CO)[C@H](O)C[C@@H]5O6)[C@H](O)C[C@@H]4O[C@H]3C[C@H]3O[C@@H](CC[C@@H]1O2)C[C@@H](C)C3=C]

or a salt or stereoisomer thereof.

### General Reaction Parameters

The following embodiments apply to all synthetic methods described herein.

The reactions provided and described herein may involve one or more reagents. In certain embodiments, a reagent may be present in a catalytic amount. In certain embodiments, a catalytic amount is from 0.001-0.1 mol%, 0.1-1 mol%, 0-5 mol%, 0-10 mol%, 1-5 mol%, 1-10 mol%, 5-10 mol%, 10-20 mol%, 20-30 mol%, 30-40 mol%, 40-50 mol%, 50-60 mol%, 60-70 mol%, 70-80 mol%, 80-90 mol%, or 90-99 mol%. In certain embodiments, a reagent may be present in a stoichiometric amount (i.e., about 1 equivalent). In certain embodiments, a reagent may be present in excess amount (i.e., greater than 1 equivalent). In certain embodiments, the excess amount is about 1.1, 1.2, 1.3, 1.4, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, or 20 equivalents. In certain embodiments, the excess amount is from about 1.1-2, 2-3, 3-4, 4-5, 1.1-5, 5-10, 10-15, 15-20, or 10-20 equivalents. In certain embodiments, the excess amount is greater than 20 equivalents.

A reaction described herein may be carried out at any temperature. In certain embodiments, a reaction is carried out at or around room temperature (rt) (around 21° C. or 70° F.). In certain embodiments, a reaction is carried out at below room temperature (e.g., from -100° C. to 21° C.). In certain embodiments, a reaction is carried out at or around -78° C. In certain embodiments, a reaction is carried out at or around -10° C. In certain embodiments, a reaction is carried out at around 0° C. In certain embodiments, a reaction is carried out at above room temperature. In certain embodiment, a reaction is carried out at 30, 40, 50, 60, 70, 80, 110, 120, 130, 140, or 150° C. In certain embodiments, a reaction is carried out at above 150° C.

A reaction described herein may be carried out in a solvent, or a mixture of solvents (i.e., cosolvents). Solvents can be polar or non-polar, protic or aprotic. Any solvent may be used in the reactions described herein, and the reactions are not limited to particular solvents or combinations of solvents. Common organic solvents useful in the methods described herein include, but are not limited to, acetone, acetonitrile, benzene, benzonitrile, 1-butanol, 2-butanone, butyl acetate, tert-butyl methyl ether, carbon disulfide carbon tetrachloride, chlorobenzene, 1-chlorobutane, chloroform, cyclohexane, cyclopentane, 1,2-dichlorobenzene, 1,2-dichloroethane, dichloromethane (DCM), N.N-dimethylacetamide N,N-dimethylformamide (DMF), 1,3-dimethyl-3,4,5,6-tetrahydro-2-pyrimidinone (DMPU), 1,4-dioxane, 1,3-dioxane, diethylether, 2-ethoxyethyl ether, ethyl acetate, ethyl alcohol, ethylene glycol, dimethyl ether, heptane, n-hexane, hexanes, hexamethylphosphoramide (HMPA), 2-methoxyethanol, 2-methoxyethyl acetate, methyl alcohol, 2-methylbutane, 4-methyl-2-pentanone, 2-methyl-1-propanol, 2-methyl-2-propanol, 1-methyl-2-pyrrolidinone, dimethylsulfoxide (DMSO), nitromethane, 1-octanol, pentane, 3-pentanone, 1-propanol, 2-propanol, pyridine, tetrachloroethylene, tetrahyrdofuran (THF), 2-methyltetrahydrofuran, toluene, trichlorobenzene, 1,1,2-trichlorotrifluoroethane, 2,2,4-trimethylpentane, trimethylamine, triethylamine, N,N-diisopropylethylamine, diisopropylamine, water, o-xylene, and p-xylene.

A reaction described herein may be carried out over any amount of time. In certain embodiments, a reaction is allowed to run for seconds, minutes, hours, or days. In certain embodiments, the Ni/Zr-mediated coupling reaction is allowed to run for seconds, minutes, hours, or days.

Methods described herein can be used to prepare compounds in any chemical yield. In certain embodiments, a compound is produced in from 1-10%, 10-20% 20-30%, 30-40%, 40-50%, 50-60%, 60-70%, 70-80%, 80-90%, or 90-100% yield. In certain embodiments, the yield is the percent yield after one synthetic step (e.g., after the Ni/Zr-mediated coupling reaction). In certain embodiments, the yield is the percent yield after more than one synthetic step (e.g., 2, 3, 4, or 5 synthetic steps).

Methods described herein may further comprise one or more purification steps. For example, in certain embodiments, a compound produced by a method described herein may be purified by chromatography, extraction, filtration, precipitation, crystallization, or any other method known in the art. In certain embodiments, a compound or mixture is carried forward to the next synthetic step without purification (i.e., crude).

In certain embodiments, a compound or reaction mixture produced by a method described herein is purified by aqueous extraction. In certain embodiments, a compound produced by a method described herein is purified by chromatography (e.g., silica gel chromatography). In certain embodiments, a compound produced by a method described herein is purified by aqueous extraction followed by chromatography (e.g., silica gel chromatography).

Metals (e.g., Ni, Zr, Zn, and/or Mn) used in the methods described herein can be removed from the reaction mixtures by one or more step of extraction, chromatography, precipitation, filtration, or any other method known in the art. In certain embodiments, a method described herein yields a product that is substantially free of metals.

The synthetic method provided herein can be carried out on any scale (i.e., to yield any amount of product). In certain embodiments, the methods are applicable to small-scale synthesis or larger-scale process manufacture. In certain embodiments, a reaction provided herein is carried out on a scale to yield less than 1 g of product. In certain embodiments, a reaction provided herein is carried out to yield greater than 1 g, 2 g, 5 g, 10 g, 15 g, 20 g, 25 g, 30 g, 40 g, 50 g, 100 g, 200 g, 500 g, or 1 kg of a product.

### Chemical Groups

The following chemical group definitions apply to all compounds and methods described herein.

### Group R^{S}

As defined herein, R^{S} is optionally substituted alkyl, optionally substituted carbocyclyl, optionally substituted aryl, optionally substituted heterocyclyl, or optionally substituted heteroaryl. In certain embodiments, R^{s} is optionally substituted alkyl. In certain embodiments, R^{s} is optionally substituted C₁₋₆alkyl. In certain embodiments, R^{s} is unsubstituted C₁₋₆ alkyl. In certain embodiments, R^{S} is selected from the group consisting of methyl, ethyl, n-propyl, iso-propyl, n-butyl, iso-butyl, sec-butyl, and tert-butyl. In certain embodiments, R^{S} is optionally substituted carbocyclyl. In certain embodiments, R^{S} is optionally substituted aryl. In certain embodiments, R^{S} is optionally substituted heterocyclyl. In certain embodiments, R^{S} is optionally substituted heteroaryl. In certain embodiments, R^{S} is optionally substituted 6-membered heteroaryl. In certain embodiments, R^{S} is optionally substituted 6-membered heteroaryl comprising 1, 2, or 3 nitrogen atoms. In certain embodiments, R^{S} is optionally substituted pyridyl. In certain embodiments, R^{S} is unsubstituted pyridyl (Py). In certain embodiments, R^{S} is optionally substituted 2-pyridyl. In certain embodiments, R^{S} is unsubstituted 2-pyridyl (2-Py). In certain embodiments, R^{S} is selected from the group consisting of:

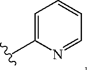

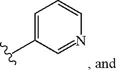

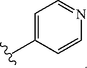

In certain embodiments, R^{S} is

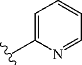

(abbreviated herein as “2-Py” or “Py”).

### Group X¹

As defined herein, X¹ is halogen or a leaving group. In certain embodiments, X¹ is a halogen. In certain embodiments, X¹ is —Cl (i.e., chloride). In certain embodiments, X¹ is -Br (i.e., bromide). In certain embodiments, X¹ is —I (i.e., iodide). In certain embodiments, X¹ is —F (i.e., fluoride). In certain embodiments, X¹ is a leaving group.

### Groups R′, R², R³, R⁴, R³, R⁶, and R⁷

As defined herein, R¹ is hydrogen, halogen, or optionally substituted alky. In certain embodiments, R¹ is hydrogen. In certain embodiments, R¹ is halogen. In certain embodiments, R¹ is optionally substituted alkyl. In certain embodiments, R¹ is optionally substituted C₁₋₆ alkyl. In certain embodiments, R¹ is unsubstituted C₁₋₆ alkyl. In certain embodiments, R¹ is optionally substituted C₁₋₃ alkyl. In certain embodiments, R¹ is unsubstituted C₁₋₃ alkyl. In certain embodiments, R¹ is selected from the group consisting of methyl, ethyl, n-propyl, iso-propyl, n-butyl, iso-butyl, see-butyl, and tert-butyl. In certain embodiments, R¹ is methyl.

As defined herein, R² is hydrogen, halogen, or optionally substituted alky. In certain embodiments, R² is hydrogen. In certain embodiments, R² is halogen. In certain embodiments, R² is optionally substituted alkyl. In certain embodiments, R² is optionally substituted C₁₋₆ alkyl. In certain embodiments, R² is unsubstituted C₁₋₆ alkyl. In certain embodiments, R² is optionally substituted C₁₋₃ alkyl. In certain embodiments, R² is unsubstituted C₁₋₃ alkyl. In certain embodiments, R² is selected from the group consisting of methyl, ethyl, n-propyl, iso-propyl, n-butyl, iso-butyl, sec-butyl, and tert-butyl. In certain embodiments, R² is methyl.

As defined herein, R³ is hydrogen, halogen, or optionally substituted alky. In certain embodiments, R³ is hydrogen. In certain embodiments, R³ is halogen. In certain embodiments, R³ is optionally substituted alkyl. In certain embodiments, R³ is optionally substituted C₁₋₆ alkyl. In certain embodiments, R³ is unsubstituted C₁₋₆ alkyl. In certain embodiments, R³ is optionally substituted C₁₋₃ alkyl. In certain embodiments, R³ is unsubstituted C₁₋₃ alkyl. In certain embodiments, R³ is selected from the group consisting of methyl, ethyl, n-propyl, iso-propyl, n-butyl, iso-butyl, sec-butyl, and tert-butyl. In certain embodiments, R³ is methyl.

As defined herein, each instance of R⁴ is independently hydrogen, halogen, or optionally substituted alkyl; and optionally two R⁴ groups are taken together to form:

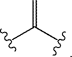

In certain embodiments, R⁴ is hydrogen. In certain embodiments, R⁴ is halogen. In certain embodiments, R⁴ is optionally substituted alkyl. In certain embodiments, R⁴ is optionally substituted C₁₋₆ alkyl. In certain embodiments, R⁴ is unsubstituted C₁₋₆ alkyl. In certain embodiments, R⁴ is optionally substituted C₁₋₃ alkyl. In certain embodiments, R⁴ is unsubstituted C₁₋₃ alkyl. In certain embodiments, R⁴ is selected from the group consisting of methyl, ethyl, n-propyl, iso-propyl, n-butyl, iso-butyl, sec-butyl, and tert-butyl. In certain embodiments, R⁴ is methyl. In certain embodiments, two R⁴ groups are taken together to form:

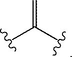

As defined herein, R⁵ is hydrogen, halogen, or optionally substituted alky. In certain embodiments, R⁵ is hydrogen. In certain embodiments, R⁵ is halogen. In certain embodiments, R⁵ is optionally substituted alkyl. In certain embodiments, R⁵ is optionally substituted C₁₋₆ alkyl. In certain embodiments, R⁵ is unsubstituted C₁₋₆ alkyl. In certain embodiments, R⁵ is optionally substituted C₁₋₃ alkyl. In certain embodiments, R⁵ is unsubstituted C₁₋₃ alkyl. In certain embodiments, R⁵ is selected from the group consisting of methyl, ethyl, n-propyl, iso-propyl, n-butyl, iso-butyl, sec-butyl, and tert-butyl. In certain embodiments, R⁵ is methyl.

As defined herein, each instance of R⁶ is independently hydrogen, halogen, or optionally substituted alkyl; and optionally two R⁶ groups are taken together to form:

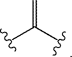

In certain embodiments, R⁶ is hydrogen. In certain embodiments, R⁶ is halogen. In certain embodiments, R⁶ is optionally substituted alkyl. In certain embodiments, R⁶ is optionally substituted C₁₋₆ alkyl. In certain embodiments, R⁶ is unsubstituted C₁₋₆ alkyl. In certain embodiments, R⁶ is optionally substituted C₁₋₃ alkyl. In certain embodiments, R⁶ is unsubstituted C₁₋₃ alkyl. In certain embodiments, R⁶ is selected from the group consisting of methyl, ethyl, n-propyl, iso-propyl, n-butyl, iso-butyl, sec-butyl, and tert-butyl. In certain embodiments, R⁶ is methyl. In certain embodiments, two R⁶ groups are taken together to form:

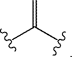

As defined herein, R⁷ is hydrogen, optionally substituted alkyl, optionally substituted carbocyclyl, optionally substituted aryl, optionally substituted heterocyclyl, optionally substituted heteroaryl, optionally substituted acyl, or an oxygen protecting group. In certain embodiments, R⁷ is hydrogen. In certain embodiments, R⁷ is optionally substituted alkyl. In certain embodiments, In certain embodiments, R⁷ is optionally substituted C₁₋₆ alkyl. In certain embodiments, R⁷ is unsubstituted C₁₋₆ alkyl. In certain embodiments, R⁷ is optionally substituted C₁₋₃ alkyl. In certain embodiments, R⁷ is unsubstituted C₁₋₃ alkyl. In certain embodiments, R⁷ is selected from the group consisting of methyl, ethyl, n-propyl, iso-propyl, n-butyl, iso-butyl, sec-butyl, and tert-butyl. In certain embodiments, R⁷ is methyl. In certain embodiments, R⁷ is ethyl. In certain embodiments, R⁷ is optionally substituted carbocyclyl. In certain embodiments, R⁷ is optionally substituted aryl. In certain embodiments, R⁷ is optionally substituted heterocyclyl. In certain embodiments, R⁷ is optionally substituted heteroaryl. In certain embodiments, R⁷ is optionally substituted acyl. In certain embodiments, R⁷ is an oxygen protecting group. In certain embodiments, R⁷ is an optionally substituted benzyl protecting group. In certain embodiments, R⁷ is benzyl (-CH₂Ph; “Bn”).

### Groups R^{X} and R^{Y}

As defined herein, R^{X} is hydrogen or -OR^{Xa}. In certain embodiments, R^{X} is hydrogen. In certain embodiments, R^{X} is -OR^{Xa}.

As generally defined herein, R^{Xa} is hydrogen, optionally substituted alkyl, optionally substituted acyl, or an oxygen protecting group. In certain embodiments, R^{Xa} is hydrogen. In certain embodiments, R^{Xa} is optionally substituted alkyl. In certain embodiments, R^{Xa} is optionally substituted acyl. In certain embodiments, R^{Xa} is or an oxygen protecting group. In certain embodiments, R^{Xa} is optionally substituted allyl. In certain embodiments, R^{Xa} is

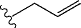

(allyl).

As defined herein, R^{Y} is hydrogen or -OR^{Ya}. In certain embodiments, R^{Y} is hydrogen. In certain embodiments, R^{Y} is -OR^{Ya}.

As generally defined herein, R^{Ya} is hydrogen, optionally substituted alkyl, optionally substituted acyl, or an oxygen protecting group. In certain embodiments, R^{Ya} is hydrogen. In certain embodiments, R^{Ya} is optionally substituted alkyl. In certain embodiments, R^{Ya} is optionally substituted acyl. In certain embodiments, R^{Ya} is or an oxygen protecting group. In certain embodiments, R^{Ya} is optionally substituted allyl. In certain embodiments, R^{Ya} is

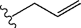

(allyl).

In certain embodiments, R^{Xa} and R^{Ya} are joined together with their intervening atoms to form optionally substituted heterocyclyl. In certain embodiments, R^{Xa} and R^{Ya} are joined together with their intervening atoms to form optionally substituted 5-membered heterocyclyl. In certain embodiments, R^{Xa} and R^{Ya} are joined together with their intervening atoms to form optionally substituted 1,3-dioxolane ring. In certain embodiments, R^{Xa} and R^{Ya} are joined together with their intervening atoms to form the following:

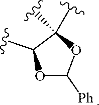

In certainembodiments, R ^{Xa} and R^{Ya}′ are joined together with their intervening atoms to form the following:

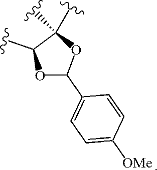

In certain embodiments, R^{X} and R^{Y} are both hydrogen.

In certain embodiments, R^{X} is hydrogen, and R^{Y} is -OR^{Ya}. In certain embodiments, R^{x} is hydrogen; and R^{Y} is —OH.

In certain embodiments, R^{X} is -OR^{Xa}; and R^{Y} is -OR^{Ya}. In certain embodiments, R^{x} is —OH; and R^{Y} is —OH.

### Groups R^{P1}, R^{P2}, R^{P3}, R^{P4}, R^{P5}and R^{P6}

As defined herein, R^{P1} is hydrogen, optionally substituted alkyl, optionally substituted acyl, or an oxygen protecting group. In certain embodiments, R^{P1} is hydrogen. In certain embodiments, R^{P1} is optionally substituted alkyl. In certain embodiments, In certain embodiments, R^{P1} is optionally substituted C₁₋₆ alkyl. In certain embodiments, R^{P1} is unsubstituted C₁₋₆ alkyl. In certain embodiments, R^{P1} is optionally substituted C₁₋₃ alkyl. In certain embodiments, R^{P1} is unsubstituted C₁₋₃ alkyl. In certain embodiments, R^{P1} is selected from the group consisting of methyl, ethyl, n-propyl, iso-propyl, n-butyl, iso-butyl, sec-butyl, and tert-butyl. In certain embodiments, R^{P1} is optionally substituted acyl. In certain embodiments, R^{P1} is an oxygen protecting group. In certain embodiments, R^{P1} is optionally substituted allyl. In certain embodiments, R^{P1} is allyl. In certain embodiments, R^{P1} is optionally substituted silyl. In certain embodiments, R^{P1} is trialkylsilyl. In certain embodiments, R^{P1} is triethylsilyl (-SiEt_{3;} “TES”). In certain embodiments, R^{P1} is trimethylsilyl (—SiMe₃; “IMS”). In certain embodiments, R^{P1} is tert-butyl dimethylsilyl (—Sit—BuMe₂; “TBS”). In certain embodiments, R^{P1} is tert-butyl diphenylsilyl (—Sit—BuPh₂; “TBDPS”). In certain embodiments, R^{P1} is an optionally substituted benzyl protecting group. In certain embodiments, R^{P1} is benzyl (—CH₂Ph; “Bn”). In certain embodiments, R^{P1} is a methoxybenzyl protecting group. In certain embodiments, R^{P1} is para-methoxybenzyl:

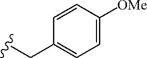

(“MPM” or “PMB”).

In certain embodiments, R^{P1} and R^{P2} are joined with the intervening atoms to form optionally substituted heterocyclyl.

As defined herein, R^{P2} is hydrogen, optionally substituted alkyl, optionally substituted acyl, or an oxygen protecting group. In certain embodiments, R^{P2} is hydrogen. In certain embodiments, R^{P2} is optionally substituted alkyl. In certain embodiments, R^{P2} is optionally substituted C₁₋₆ alkyl. In certain embodiments, R^{P2} is unsubstituted C₁₋₆ alkyl. In certain embodiments, R^{P2} is optionally substituted C₁₋₃ alkyl. In certain embodiments, R^{P2} is unsubstituted C₁₋₃ alkyl. In certain embodiments, R^{P2} is selected from the group consisting of methyl, ethyl, n-propyl, iso-propyl, n-butyl, iso-butyl, sec-butyl, and tert-butyl. In certain embodiments, R^{P2} is optionally substituted acyl. In certain embodiments, R^{P2} is an oxygen protecting group. In certain embodiments, R^{P2} is optionally substituted allyl. In certain embodiments, R^{P2} is allyl. In certain embodiments, R^{P2} is optionally substituted silyl. In certain embodiments, R^{P2} is trialkylsilyl. In certain embodiments, R^{P2} is triethylsilyl (—SiEt₃; “TES”). In certain embodiments, R^{P2} is trimethylsilyl (—SiMe₃; “TMS”). In certain embodiments, R^{P2} is tert-butyl dimethylsilyl (—Sit—BuMe₂; “TBS”). In certain embodiments, R^{P2} is tert-butyl diphenylsilyl (—Sit—BuPh₂; “TBDPS”). In certain embodiments, R^{P2} is an optionally substituted benzyl protecting group. In certain embodiments, R^{P2} is benzyl (—CH₂Ph; “Bn”). In certain embodiments, R^{P2} is a methoxybenzyl protecting group. In certain embodiments, R^{P2} is para-methoxybenzyl:

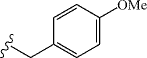

(“MPM” or “PMB”).

In certain embodiments, R^{P3} and R^{P3} are joined with the intervening atoms to form optionally substituted heterocyclyl.

As defined herein, R^{P3} is hydrogen, optionally substituted alkyl, optionally substituted acyl, or an oxygen protecting group. In certain embodiments, R^{P3} is hydrogen. In certain embodiments, R^{P3} is optionally substituted alkyl. In certain embodiments, R^{P3} is optionally substituted C₁₋₆ alkyl. In certain embodiments, R^{P3} is unsubstituted C₁₋₆ alkyl. In certain embodiments, R^{P3} is optionally substituted C₁₋₃ alkyl. In certain embodiments, R^{P3} is unsubstituted C₁₋₃ alkyl. In certain embodiments, R^{P3} is selected from the group consisting of methyl, ethyl, n-propyl, iso-propyl, n-butyl, iso-butyl, see-butyl, and tert-butyl. In certain embodiments, R^{P3} is optionally substituted acyl. In certain embodiments, R^{P3} is an oxygen protecting group. In certain embodiments, R^{P3} is optionally substituted allyl. In certain embodiments, R^{P3} is allyl. In certain embodiments, R^{P3} is optionally substituted silyl. In certain embodiments, R^{P3} is trialkylsilyl. In certain embodiments, R^{P3} is triethylsilyl (—SiEt₃; “TES”). In certain embodiments, R^{P3} is trimethylsilyl (—SiMe₃; “TMS”). In certain embodiments, R^{P3} is tert-butyl dimethylsilyl (—Sit—BuMe₂; “TBS”). In certain embodiments, R^{P3} is tert-butyl diphenylsilyl (—Sit—BuPh₂; “TBDPS”). In certain embodiments, R^{P3} is an optionally substituted benzyl protecting group. In certain embodiments, R^{P3} is benzyl (—CH₂Ph; “Bn”). In certain embodiments, R^{P3} is a methoxybenzyl protecting group. In certain embodiments, R^{P1} is para-methoxybenzyl:

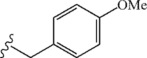

(“MPM” or “PMB”).

As defined herein, R^{P4} is hydrogen, optionally substituted alkyl, optionally substituted acyl, or an oxygen protecting group. In certain embodiments, R^{P4} is hydrogen. In certain embodiments, R^{P4} is optionally substituted alkyl. In certain embodiments, R^{P4} is optionally substituted C₁₋₆ alkyl. In certain embodiments, R^{P4} is unsubstituted C₁₋₆ alkyl. In certain embodiments, R^{P4} is optionally substituted C₁₋₃ alkyl. In certain embodiments, R^{P4} is unsubstituted C₁₋₃ alkyl. In certain embodiments, R^{P4} is selected from the group consisting of methyl, ethyl, n-propyl, iso-propyl, n-butyl, iso-butyl, sec-butyl, and tert-butyl. In certain embodiments, R^{P4} is optionally substituted acyl. In certain embodiments, R^{P4} is an oxygen protecting group. In certain embodiments, R^{P4} is optionally substituted allyl. In certain embodiments, R^{P4} is allyl. In certain embodiments, R^{P4} is optionally substituted silyl. In certain embodiments, R^{P4} is trialkylsilyl. In certain embodiments, R^{P4} is triethylsilyl (—SiEt₃; “TES”). In certain embodiments, R^{P4} is trimethylsilyl (—SiMe₃; “TMS”). In certain embodiments, R^{P4} is tert-butyl dimethylsilyl (—Sit—BuMe₂; “TBS”). In certain embodiments, R^{P4} is tert-butyl diphenylsilyl (—Sit—BuPh₂; “TBDPS”). In certain embodiments, R^{P4} is an optionally substituted benzyl protecting group. In certain embodiments, R^{P4} is benzyl (—CH₂Ph; “Bn”). In certain embodiments, R^{P4} is a methoxybenzyl protecting group. In certain embodiments, R^{P4} is para-methoxybenzyl:

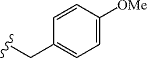

(“MPM” or “PMB”).

As defined herein, R^{P5} is hydrogen, optionally substituted alkyl, optionally substituted acyl, or an oxygen protecting group; optionally wherein two R^{P5} are joined with the intervening atoms to form optionally substituted heterocyclyl. In certain embodiments, R^{P5} is hydrogen. In certain embodiments, R^{P5} is optionally substituted alkyl. In certain embodiments, R^{P5} is optionally substituted C₁₋₆ alkyl. In certain embodiments, R^{P5} is unsubstituted C₁₋₆ alkyl. In certain embodiments, R^{P5} is optionally substituted C₁₋₃ alkyl. In certain embodiments, R^{P5} is unsubstituted C₁₋₃ alkyl. In certain embodiments, R^{P5} is selected from the group consisting of methyl, ethyl, n-propyl, iso-propyl, n-butyl, iso-butyl, sec-butyl, and tert-butyl. In certain embodiments, R^{P5} is optionally substituted acyl. In certain embodiments, R^{P5} is an oxygen protecting group. In certain embodiments, R^{P5} is optionally substituted allyl. In certain embodiments, R^{P5} is allyl. In certain embodiments, R^{P5} is optionally substituted silyl. In certain embodiments, R^{P5} is trialkylsilyl. In certain embodiments, R^{P5} is triethylsilyl (—SiEt₃; “TES”). In certain embodiments, R^{P5} is trimethylsilyl (—SiMe₃; “TMS”). In certain embodiments, R^{P5} is tert-butyl dimethylsilyl (—Sit—BuMe₂; “TBS”). In certain embodiments, R^{P5} is tert-butyl diphenylsilyl (—Sit—BuPh₂; “TBDPS”). In certain embodiments, R^{P5} is an optionally substituted benzyl protecting group. In certain embodiments, R^{P5} is benzyl (—CH₂Ph; “Bn”). In certain embodiments, R^{P5} is a methoxybenzyl protecting group. In certain embodiments, R^{P5} is para-methoxybenzyl:

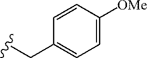

(“MPM” or “PMB”). In certain embodiments, two R^{P5} are joined with the intervening atoms to form optionally substituted heterocyclyl. In certain embodiments, two R^{P5} are joined with the intervening atoms to form optionally substituted six-membered heterocyclyl. In certain embodiments, two R^{P5} are joined with the intervening atoms to form a ring of the formula:

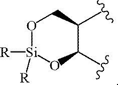

In certain embodiments, two R^{P5} are joined with the intervening atoms to form a ring of the formula:

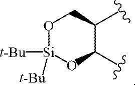

In certain embodiments, two R^{P5} are joined with the intervening atoms to form a ring of the formula:

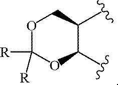

In certain embodiments, two R^{P5} are joined with the intervening atoms to form a ring of the formula:

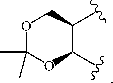

As defined herein, R^{P6} is hydrogen, optionally substituted alkyl, optionally substituted acyl, or an oxygen protecting group; optionally wherein two R^{P6} are joined with the intervening atoms to form optionally substituted heterocyclyl. In certain embodiments, R^{P6} is hydrogen. In certain embodiments, R^{P6} is optionally substituted alkyl. In certain embodiments, R^{P6} is optionally substituted C₁₋₆ alkyl. In certain embodiments, R^{P6} is unsubstituted C₁₋₆ alkyl. In certain embodiments, R^{P6} is optionally substituted C₁₋₃ alkyl. In certain embodiments, R^{P6} is unsubstituted C₁₋₃ alkyl. In certain embodiments, R^{P6} is selected from the group consisting of methyl, ethyl, n-propyl, iso-propyl, n-butyl, iso-butyl, sec-butyl, and tert-butyl. In certain embodiments, R^{P6} is optionally substituted acyl. In certain embodiments, R^{P6} is an oxygen protecting group. In certain embodiments, R^{P6} is optionally substituted allyl. In certain embodiments, R^{P6} is allyl. In certain embodiments, R^{P6} is optionally substituted silyl. In certain embodiments, R^{P6} is trialkylsilyl. In certain embodiments, R^{N} is triethylsilyl (—SiEt₃; “TES”). In certain embodiments, R^{P6} is trimethylsilyl (—SiMe₃; “TMS”). In certain embodiments, R^{P6} is tert-butyl dimethylsilyl (—Sit—BuMe₂; “TBS”). In certain embodiments, R^{P6} is tert-butyl diphenylsilyl (—Sit—BuPh₂; “TBDPS”). In certain embodiments, R^{P6} is an optionally substituted benzyl protecting group. In certain embodiments, R^{P6} is benzyl (—CH₂Ph; “Bn”). In certain embodiments, R^{P6} is a methoxybenzyl protecting group. In certain embodiments, R^{P6} is para-methoxybenzyl:

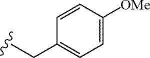

(“MPM” or “PMB”). In certain embodiments, two R^{P6} are joined with the intervening atoms to form optionally substituted heterocyclyl. In certain embodiments, two R^{P6} are joined with the intervening atoms to form optionally substituted six-membered heterocyclyl. In certain embodiments, two R^{P6} are joined with the intervening atoms to form a ring of the formula:

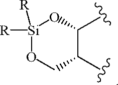

In certain embodiments, two R^{P6} are joined with the intervening atoms to form a ring of the formula:

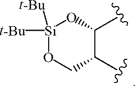

In certain embodiments, R^{P1}, R^{P2}, R^{P3}, R^{P4} and R^{P5} are silyl protecting groups. In certain embodiments, R^{P1} and R^{P2} are TBS; and R^{P3}, R^{P4}, and R^{P5} are TES.

In certain embodiments, R^{P3} is a silyl protecting group; R⁷ is optionally substituted alkyl, and R^{P4} and R^{P5} are silyl protecting groups. In certain embodiments, R^{P3} is TES; R⁷ is methyl; and R^{P4} and R^{P5} are TES.

In certain embodiments, two R^{P6} are joined to form:

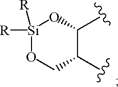

and R^{P4} and R^{P5} are silyl protecting groups. In certain embodiments, two R^{P6} are joined to form:

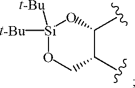

and R^{P4} and R^{P5} are TES.

### Group R

As generally defined herein, each R is independently hydrogen or optionally substituted alkyl. In certain embodiments, at least one instance of R is hydrogen. In certain embodiments, at least one instance of R is optionally substituted alkyl. In certain embodiments, at least one instance of R is optionally substituted C₁₋₆ alkyl. In certain embodiments, at least one instance of R is unsubstituted C₁₋₆ alkyl. In certain embodiments, at least one instance of R is optionally substituted C₁₋₃ alkyl. In certain embodiments, at least one instance of R is unsubstituted C₁₋₃ alkyl. In certain embodiments, at least one instance of R is selected from the group consisting of methyl, ethyl, n-propyl, iso-propyl, n-butyl, iso-butyl, sec-butyl, and tert-butyl. In certain embodiments, each R is tert-butyl.

### Group X, R^{c}, R^{c′}, R^{N}, p, and s

As defined herein, each instance of X is independently halogen. In certain embodiments, each X is —Cl. In certain embodiments, each X is —Br. In certain embodiments, each X is —I. In certain embodiments, each X is —F.

As defined herein, each instance of p is independently 0 or an integer from 1-4, inclusive. In certain embodiments, p is 0. In certain embodiments, p is 1. In certain embodiments, p is 2. In certain embodiments, p is 3. In certain embodiments, p is 4. In certain embodiments, p is 5.

As defined herein, s is 0, 1, or 2. In certain embodiments, s is 0. In certain embodiments, s is 1. In certain embodiments, s is 2.

As defined herein, each instance of R^{c} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1}. In certain embodiments, at least one instance of R^{c} is hydrogen. In certain embodiments, at least one instance of R^{c} is halogen In certain embodiments, at least one instance of R^{c} is —CN. In certain embodiments, at least one instance of R^{c} is —NO₂. In certain embodiments, at least one instance of R^{c} is —N₃. In certain embodiments, at least one instance of R^{c} is optionally substituted alkyl. In certain embodiments, at least one instance of R^{c} is optionally substituted alkenyl. In certain embodiments, at least one instance of R^{c} is optionally substituted alkynyl. In certain embodiments, at least one instance of R^{c} is optionally substituted aryl. In certain embodiments, at least one instance of R^{c} is optionally substituted heteroaryl. In certain embodiments, at least one instance of R^{c} is optionally substituted carbocyclyl. In certain embodiments, at least one instance of R^{c} is optionally substituted heterocyclyl. In certain embodiments, at least one instance of R^{c} is optionally substituted acyl. In certain embodiments, at least one instance of R^{c} is —N(R^{N})₂. In certain embodiments, at least one instance of R^{c} is -OR^{O}. In certain embodiments, at least one instance of R^{c} is or -SR^{S1}. In certain embodiments, at least one instance of R^{c} is optionally substituted C₁₋₆ alkyl. In certain embodiments, at least one instance of R^{c} is unsubstituted C₁₋₆ alkyl. In certain embodiments, at least one instance of R^{c} is optionally substituted C₁₋₃ alkyl. In certain embodiments, at least one instance of R^{c} is unsubstituted C₁₋₃ alkyl. In certain embodiments, at least one instance of R^{c} is selected from the group consisting of methyl, ethyl, n-propyl, iso-propyl, n-butyl, iso-butyl, see-butyl, and tert-butyl. In certain embodiment, at least one instance of R^{c} is methyl. In certain embodiment, each instance of R^{c} is methyl.

As defined herein, each instance of R^{c′} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1}. In certain embodiments, at least one instance of R^{c′} is hydrogen. In certain embodiments, at least one instance of R^{c′} is halogen In certain embodiments, at least one instance of R^{c′} is —CN. In certain embodiments, at least one instance of R^{c′} is —NO₂. In certain embodiments, at least one instance of R^{c′} is —N₃. In certain embodiments, at least one instance of R^{c′} is optionally substituted alkyl. In certain embodiments, at least one instance of R^{c′} is optionally substituted alkenyl. In certain embodiments, at least one instance of R^{c′} is optionally substituted alkynyl. In certain embodiments, at least one instance of R^{c′} is optionally substituted aryl. In certain embodiments, at least one instance of R^{c′} is optionally substituted heteroaryl. In certain embodiments, at least one instance of R^{c′} is optionally substituted carbocyclyl. In certain embodiments, at least one instance of R^{c′} is optionally substituted heterocyclyl. In certain embodiments, at least one instance of R^{c′} is optionally substituted acyl. In certain embodiments, at least one instance of R^{c′} is —N(R^{N})₂. In certain embodiments, at least one instance of R^{c′} is -OR^{O}. In certain embodiments, at least one instance of R^{c′} is or -SR^{S1.}

As defined herein, each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or two R^{N} bonded to the same nitrogen atom are taken together with the intervening atoms to form optionally substituted heterocyclyl or optionally substituted heteroaryl. In certain embodiments, at least one instance of R^{N} is optionally substituted alkyl. In certain embodiments, at least one instance of R^{N} is optionally substituted alkenyl. In certain embodiments, at least one instance of R^{N} is optionally substituted alkynyl. In certain embodiments, at least one instance of R^{N} is optionally substituted aryl. In certain embodiments, at least one instance of R^{N} is optionally substituted heteroaryl. In certain embodiments, at least one instance of R^{N} is optionally substituted carbocyclyl. In certain embodiments, at least one instance of R^{N} is optionally substituted heterocyclyl. In certain embodiments, at least one instance of R^{N} is optionally substituted acyl. In certain embodiments, at least one instance of R^{N} is a nitrogen protecting group. In certain embodiments, at least one instance of R^{N} is optionally substituted C₁₋₆ alkyl. In certain embodiments, at least one instance of R^{N} is unsubstituted C₁₋₆ alkyl. In certain embodiments, at least one instance of R^{N} is optionally substituted C₁₋₃ alkyl. In certain embodiments, at least one instance of R^{N} is unsubstituted C₁₋₃ alkyl. In certain embodiments, at least one instance of R^{N} is selected from the group consisting of methyl, ethyl, n-propyl, iso-propyl, n-butyl, iso-butyl, sec-butyl, and tert-butyl. In certain embodiment, at least one instance of R^{N} is methyl.

each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group. In certain embodiments, at least one instance of R^{O} is optionally substituted alkyl. In certain embodiments, at least one instance of R^{O} is optionally substituted alkenyl. In certain embodiments, at least one instance of R^{O} is optionally substituted alkynyl. In certain embodiments, at least one instance of R^{O} is optionally substituted aryl. In certain embodiments, at least one instance of R^{O} is optionally substituted heteroaryl. In certain embodiments, at least one instance of R^{O} is optionally substituted carbocyclyl. In certain embodiments, at least one instance of R^{O} is optionally substituted heterocyclyl. In certain embodiments, at least one instance of R^{O} is optionally substituted acyl. In certain embodiments, at least one instance of R^{O} is an oxygen protecting group.

each instance of R^{S1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group. In certain embodiments, at least one instance of R^{S1} is optionally substituted alkyl. In certain embodiments, at least one instance of R^{S1} is optionally substituted alkenyl. In certain embodiments, at least one instance of R^{S1} is optionally substituted alkynyl. In certain embodiments, at least one instance of R^{S1} is optionally substituted aryl. In certain embodiments, at least one instance of R^{S1} is optionally substituted heteroaryl. In certain embodiments, at least one instance of R^{S1} is optionally substituted carbocyclyl. In certain embodiments, at least one instance of R^{S1} is optionally substituted heterocyclyl. In certain embodiments, at least one instance of R^{S1} is optionally substituted acyl. In certain embodiments, at least one instance of R^{S1} is a sulfur protecting group.

### EXAMPLES

### Ni./Zr-Mediated Coupling Reactions

A new Ni/Zr-mediated one-pot ketone synthesis was developed with use of a mixture of (Me)₃tpy—Ni^{I}I— and py-(Me)imid·N^{II}Cl₂-catalysts. The Ni^{I}-catalyst selectively activates alkyl halides, whereas the Ni^{II}-catalyst activates thioesters. An adjustment of a relative loading of the two catalysts allows to tune the relative rate of the two activations and trap a short-lived radical intermediate(s) efficiently. Thus, the new method makes one-pot ketone synthesis highly effective even with a 1:1 mixture of the coupling partners. The synthetic value of the new method is demonstrated with the C—C bond formation at the final stage of a convergent halichondrin-synthesis.

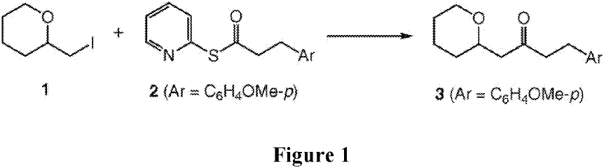

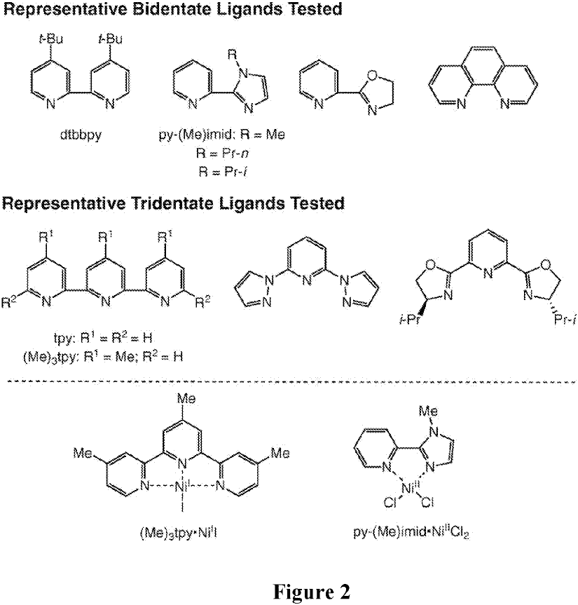

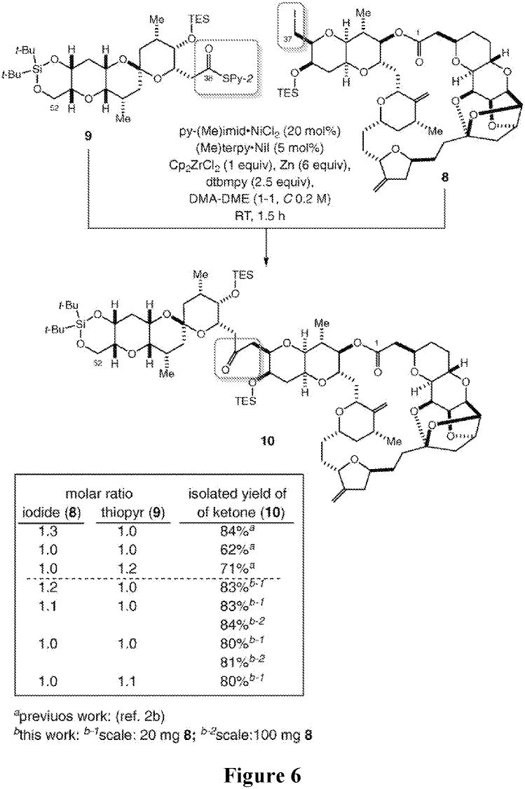

Recently, a Ni/Zr-mediated one-pot ketone synthesis was reported, which was successfully applied to a unified total synthesis of the halichondrin class of marine natural products.^{1,2} During a study of extending the one-pot ketone synthesis to macroketocyclization, it was observed that the efficiency of cyclization was noticeably improved with use of a mixture of (dtbbpy)·NiBr₂ and (tpy)-NiCl₂ (FIG. 2).³ This observation led to the examination of a mixture of Ni^{I}— and Ni^{II}-catalysts for one-pot ketone synthesis. The development and application of a new one-pot ketone synthesis using a mixture of py(Me)imid·Ni^{II}Cl₂- and (Me)₃tpy·Ni^{I}I-catalysts is reported herein. The ketone coupling of 1 + 2 → 3 has been used for development (FIG. 1), whereas the ketone coupling of 8 + 9 → 10 in the halichondrin series has been used for application (FIG. 6).

With the hope of identifying better catalysts, various NiX₂-complexes with bidentate- and tridentate-ligands were first screened. Through this screen, 2-(1-methyl-1H-imidazol-2-yl)pyridine (abbreviated as py-(Me)imid) and 4,4′,4″-trimethyl-2,2′:6′,2″-terpyridine (abbreviated as (Me)₃-tpy) emerged as the best-performing ligands (FIG. 2). It was speculated that the effectiveness of NiX₂-complexes with tridentate-ligands may suggest that a Ni^{I}-species plays a key role for the observed phenomenon. Adopting the procedure reported by Vicic,⁴ (Me)3tpy-Ni^{I}I was prepared and tested, thereby demonstrating that the Ni^{I}-complex indeed gave better results than (Me)₃tpy·NiCl₂. For this reason, (Me)₃tpy·Ni^{I}I and py-(Me)imid·Ni^{II}Cl₂ has been used for the following studies.

The coupling was effected by either Zn— or Mn-metal in the presence of Cp₂ZrCl₂ (0.7-1.0 equiv), although the coupling with Mn-metal is slower than that with Zn-metal. A 1:1 mixture of N.N-dimethylacetamide (DMA) and 1,2-dimethoxyethane (DME) at C ~0.5 M was used as a solvent, and the coupling was typically completed within 3 hours at RT.^{5,6}

The ketone coupling of 1 + 2 → 3 requires the activation of the iodide in 1 as well as the thiopyridine ester in 2. In the previous method, (dtbbpy)·NiBr₂ activates obviously both iodide and thiopyridine ester. The results in Table 1 demonstrate that (Me)3tpy·Ni^{I}I selectively activates the iodide 1, whereas py-(Me)imid·Ni^{II}Cl₂ activates thiopyridine ester 2. However, it should be noted that the Ni^{II}-catalyst also activates 1, but the rate of activation with the Ni^{II}-catalyst is significantly slower than that with the Ni^{II}-catalyst.

TABLE 1

Studies to determine the primary role of the two catalysts.
1 + 2 → 3 (Ar = C₈H₄OM_{e-P}) (1.0 equiv) (1.0 equiv)

| | (Me)₃tpy· Ni | (py-(Me)imid)· NiCl₂ | time (h) | recovered 1 | recovered 2 | isolated 3 |
|---|---|---|---|---|---|---|
| entry 1 | 1 mol% | 1 mol% | 3 | 0% | 0% | 96% |
| entry 2 | 2 mol% | 0 | 0.5 | 0% | 70% | 22% |
| entry 3 | 2 mol% | 0 | 3 | 0% | 65% | 22% |
| entry 4 | 0 | 2 mol% | 0.5 | 51% | 44% | 21% |
| entry 5 | 0 | 2 mol% | 3 | 26% | 12% | 46% |

Reactions were carried out with or without Ni— and Ni — catalysts in the presence of Cp₂ZrCl₂ (1 equiv) and Zn (3 equiv) in DMA/DME (1/1, 0 0.5 M) at RT. The amounts indicated for 1-3 are based on the materials isolated chromatographically.

In the absence of Zn-metal, 1 is stable against (Me)₃tpy·Ni^{I}I, thereby indicating that the activation of 1 requires a Ni⁰-species generated from (Me)₃terpy-Ni^{I}I in situ. Indeed, the coupling of 1 + 2 - 3 with Ni⁰-species, prepared from Ni⁰(COD)₂ and (Me)₂tpy ligand, gave ketone 3.⁶ It is worth noting that iodide 1 gave two products 4 and 5 under the condition of (Me)₃tpy·Ni^{I}I/Zn (Entry 3) but only 4 under the condition of Ni⁰(Me)₂tpy, showing that 4 is formed via dimerization of the radical, whereas 5 via the reductive ring-opening 1 with Zn-metal.

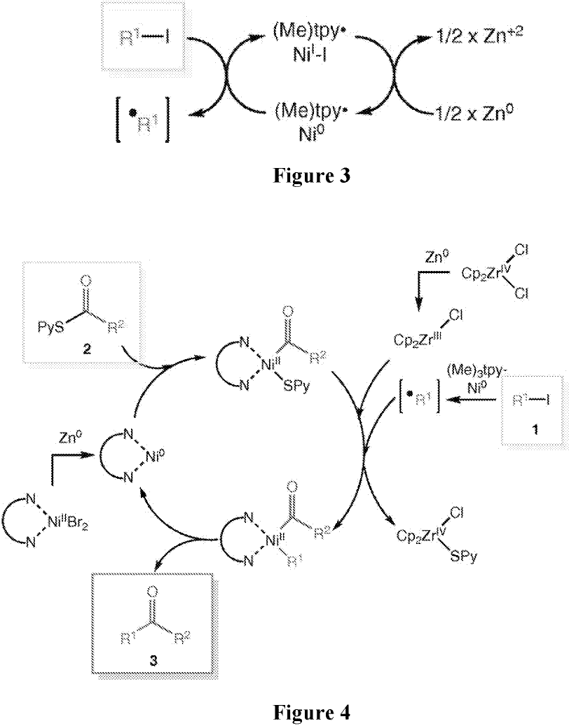

Overall, (Me)₃tpy·Ni^{I}—I is first reduced by Zn— or Mn-metal to the corresponding (Me)₃tpy·Ni⁰ and then serves as a radical initiator for the ketone coupling (FIG. 3).⁷ However, recent reports on the chemistry of Ni^{I}-complexes may suggest that this process is more complex than presented.⁸

The primary role of py-(Me)imid·Ni^{II}Cl₂ is the activation of 2 via the well-precedent oxidative addition with the Ni⁰-species generated from the Ni^{II}-complex and Zn-metal in situ (FIG. 4). Once again, the experiment with Ni⁰-species prepared from Ni⁰(COD)₂ and py-(Me)imid supported this view.

Interestingly, the activation of both 1 and 2 is affected by a Ni⁰-species, yet in a different mode. The observed selectivity is attributed to the structural difference between py-(Me)imid·Ni— and (Me)₃tpy·Ni-complexes; it is known that the Ni-complex with a bidentate ligand adopts a tetrahedral structure, whereas the Ni-complex with a terpyridine ligand adopts a squire-planar structure.⁹

Among Ni^{II}Br₂- or Ni^{II}Cl₂-complexes with bidentate-ligands, py-(Me)imid·Ni^{II}Cl₂ was found to be the fastest and cleanest catalyst thus far. In addition, the activated species generated from 2 was found stable at least 0.5-1 hours in the reaction system.¹⁰

As reported previously, zirconocene greatly accelerates the rate of coupling. ^{2a} A remarkable rate-enhancement is not observed with Cp₂Zr^{IV}Cl₂ alone, but with Cp₂Zr^{IV}Cl₂ with Zn-metal, thereby suggesting that it is caused by a reduced form of Cp₂Zr^{IV}Cl₂, and it is speculated to be Cp₂Zr^{III}Cl.¹¹

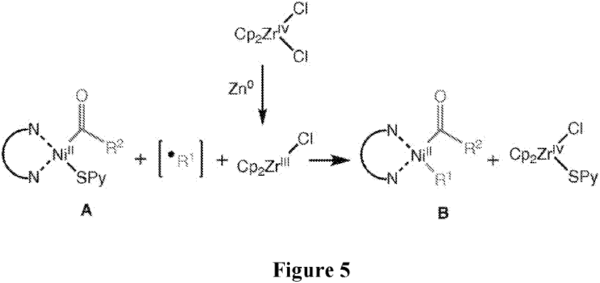

Obviously, a ligand-exchange is required to form ketone 3 from the initially formed oxidative-addition intermediate, i.e., A → B (FIG. 5). Interestingly, this ligand-exchange needs to take place with the afore-mentioned radical [^{•}R]. Without wishing to be bound by any particular theory, it is assumed that this ligand exchange is accelerated by Cp₂Zr^{III}Cl via forming a strong Zr^{IV}—S bond and (simultaneously) weakening a Ni^{II}—S bond. It is worth noting that phenylthio esters, corresponding to 2, also serve as electrophiles in the coupling.¹²

This new method has great synthetic value. As mentioned, the ketone coupling of 1 + 2 → 3 requires the activation of iodide 1 and thiopyridine ester 2. In the previous method, (dtbbpy)·NiBr₂ activates both iodide and thiopyridine ester. Therefore, the relative rate of two activations is inherent to a given pair of coupling partners. In the new method, the two activations are independently effected by two catalysts (Me)₃tpy·Ni^{I}I and py-(Me)imid·Ni^{II}Cl₂, thereby allowing us to tune the relative rates of the two activations by adjusting a relative loading of two catalysts.

A number of substrates tested in this laboratory show that activation of an iodide by (Me)₃tpy·Ni^{I}I is uniformly fast, but the generated radicals are short-lived.¹³ Contrarily, it has been observed that the rate of thiopyridine-ester activation widely varies, depending on substrate structure. Fortunately, generated intermediates are relatively long-lived.¹⁰ Therefore, it is now possible to tune the relative rate of the two activations by simply adjusting the relative loading of the two catalysts to trap the short-lived radical intermediates by A. In order to trap the radical intermediate without waste, it is necessary to keep the relative rate of activation of an iodide slower than that of a thiopyridine ester. For the case of 1 + 2 → 3, the isolated yield reaches a plateau at around 3/1 ratio of the Ni^{I}— and Ni^{II}-catalyst-loadings, cf., Entry 5 vs. Entries 1-4 (Table 2). Interestingly, it has been observed that this ratio varies widely, depending on substrate structure. In general, as the rate of thiopyridine-ester activation is slower for a complex substrate, a lower ratio of Ni^{I}-over Ni^{II}-catalyst-loadings gives a better coupling yield. For example, the Ni^{I}/Ni^{II} = ¼ was used for the halichondrin case (FIG. 6).

TABLE 2

Ratio of Ni^{I}— and Ni^{II}-catalyst-loadings and coupling yields.
1 + 2 → 3 (1.0 equiv) (1.0 equiv)

| | (Me)₃tpy•Ni^{I}l | py-(Me)imid-Ni^{II}Cl₂ | isolated ketone |
|---|---|---|---|
| entry 1 | 1 mol% | 5 mol% | 96% |
| entry 2 | 1 mol% | 3 mol% | 96% |
| entry 3 | 1 mol% | 1 mol% | 96% |
| entry 4 | 3 mol% | 1 mol% | 96% |
| entry 5 | 5 mol% | 1 mol% | 91% |

Ketone-coupling was done with (Me)₃tpy•Nil, py-(Me)imid• NiCl_{2,} Cp₂ZrCl₂ (1 equiv), and Zn (3 equiv) in DMA-DME (1:1, C 0.5 M) at RT, 3 h.

With this knowledge, it is now possible to realize the one-pot ketone synthesis efficiently even with a 1:1 ratio of the coupling partners. The results summarized in Table 3 and FIG. 6 (vide infra) support this claim.

TABLE 3

Ratio of substrates 1 and 3 and coupling yields.
1 + 2 → 3

| | 1 (equiv) | 2 (equiv) | isolated ketone |
|---|---|---|---|
| entry 1 | 1.1 | 1.0 | 96% |
| entry 2 | 1.0 | 1.0 | 96% |
| entry 3 | 1.0 | 1.1 | 94% |

Ketone-coupling was done with (Me)₃tpy•Ni^{I}I (1 mol%), py(Me)imid• Ni^{II}Cl₂ (1 mol%), Cp₂ZrCl₂ (1 equiv), and Zn (3 equiv) in DMA-DME (1:1, C 0.5 M) at RT, 3 h.

In addition, the overall coupling-rate can be controlled by the loading of the two catalysts. For example, the coupling of 1 + 2 → 3 was completed in ~3 hours, with 1 mol % loading of the two catalysts.

The new method shows the scope and limitation very similar to those observed for the previous method (Table 4).¹⁴ However, it should be noted that comparable, or even slightly better, yields were achieved with the new method with use of a 1:1 mixture of the iodide and thiopyridine ester, as opposed to a 1.2:1 mixture in the previous method.

TABLE 4

Selected examples of ketone couplings

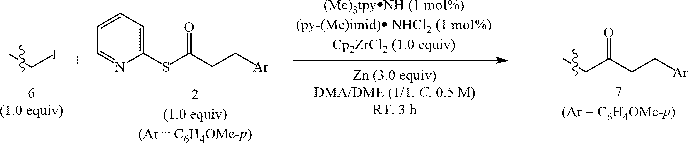

a. lodides bearing a leaving group at β-position

| 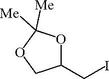 | 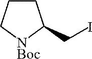 [SMILES: CC(C)(C)OC(=O)N1CCC[C@H]1CI] | 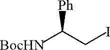 [SMILES: CC(C)(C)OC(=O)N[C@H](CI)c1ccccc1] | 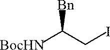 [SMILES: CC(C)(C)OC(=O)N[C@H](CI)Cc1ccccc1] |
|---|---|---|---|
| 6a: 96% | 6b: 90% | 6c: 96% | 6d: 93% |
| b. Steric hindrance tolerance | | | |
| 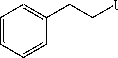 | 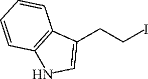 [SMILES: ICCc1c[nH]c2ccccc12] | 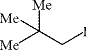 | 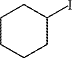 |
| 6e: 92% | 6f: 90% | 6 g: 90% | 6h: 90% |
| c. Halide differentiation | | | |
| 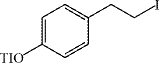 | 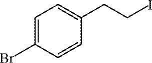 | 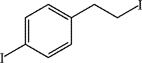 | 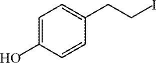 |
| 6i: 84% | 6j: 83% | 6k: 61% | 6l: 85% |

It is well appreciated that a convergent approach is the choice for a synthesis of a complex molecule over a linear approach. In order to carry out a convergent synthesis effectively, an efficient coupling reaction(s) is required. However, only a limited number of coupling reactions are shown to be useful at the late stage of a complex molecule synthesis; ideal coupling reactions need to meet a number of demanding criteria, including functional group tolerance, coupling efficiency with a ~1:1 molar ratio of coupling partners, coupling rate, stereoselectivity, and others. The previous one-pot ketone synthesis was successfully applied to the final C—C bond formation in the unified synthesis of halichondrins, although 1.0:1.3 molar ratio of coupling partners were required to achieve >80% yields.^{2b} As shown in FIG. 6, the new method gives an excellent solution to improve the molar ratio of 8 and 9. Note that a 4:1 mixture of the two catalysts was used for this case, because the activation of 9 was slow, compared with that of 8.

In summary, a new Ni/Zr-mediated one-pot ketone synthesis has been developed, with use of a mixture of (Me)₃tpy·Ni^{I}I- and py-(Me)imid·Ni^{II}Cl₂-catalysts. The Ni^{I}-catalyst selectively activates iodides, whereas the Ni^{II}-catalyst activates thiopyridine esters. An adjustment of the relative loading of the two catalysts allows us to tune the relative rate of the two activations and trap a short-lived radical intermediate(s) efficiently. The new method is efficient, even with use of a 1:1 mixture of the coupling partners. The synthetic value of the new method is demonstrated with the C—C bond formation at the final stage of a convergent halichondrin synthesis.

### General Experimental Information

Unless otherwise noted, all reagents and solvents were obtained from commercial suppliers and used without further purification. Reactions involving air or moisture sensitive reagents or intermediates were performed under an inert atmosphere of nitrogen or argon in glassware that was oven dried. Analytical thin layer chromatography (TLC) was performed with E. Merck precoated TLC plates, silica gel 60F-254, layer thickness 0.25 mm. TLC plates were visualized by staining with AMCAN (ammonium molybdate/cerium ammonium nitrate), PMA (phosphomolybdic acid hydrate), or p-anisaldehyde. Flash chromatography separations were performed on E. Merck Silica Gel 60 (40-63 µm), Kanto Chemical Silica Gel 60N (spherical, neutral, 40-50 µm), or Wako Pure Chemical Industry Wakogel 50NH₂ (38-63 µm). Medium pressure column chromatography was performed with YAMAZEN Smart Flash. NMR spectra were recorded on a Varian Inova 600 MHz or Varian Inova 500 MHz. Chemical shifts were reported in parts per million (ppm). The residual solvent peak was used as an internal reference (for ¹H NMR spectra: 7.26 ppm in CDCl₃, 7.16 ppm in C₆D₆; for ¹³C NMR: 77.0 ppm in CDCl₃ and 128.0 ppm in C₆D₆). Coupling constants (J) are reported in Hz and the splitting abbreviations used are: s for singlet, d for doublet, t for triplet, q for quartet, m for multiplet, and br for broad. Optical rotations were measured at 20° C. using Perkin-Elmer 241 polarimeter. IR spectra were recorded on Bruker Alpha FT-IR spectrometer. Electrospray ionization experiments were performed on Micromass Inc., Platform II Atmospheric Pressure Ionization Mass Spectrometer.

### Preparation of Nickel Catalysts

### Py-(Me)imid-Ni^{II}Cl₂

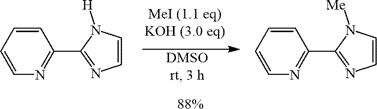

To a stirred solution of 2-(1H-imidazol-2-yl)pyridine (Aldrich; 4.00 g, 27.6 mmol) in DMSO (40 mL) was added KOH fine powder (4.64 g, 82.8 mmol) at room temperature. After stirring for 30 minutes, MeI (1.90 mL, 30.4 mmol) was added to a mixture at 0° C. The reaction mixture was allowed to warm up to room temperature and stirred for 3 hours. The reaction mixture was poured into water (160 mL), and the resulting mixture was extracted with CH₂Cl₂ (80 mL) three times. The combined organic extracts were washed with water (120 mL) five times and brine, dried over anhydrous Na₂SO₄, filtered, and concentrated under reduced pressure. The residue was purified by flash silica gel column chromatography (CH₂Cl₂/MeOH = 20:1) to afford 2—(Me—imidazol-2-yl)pyridine (3.91 g, 24.3 mmol, 88%) as a colorless oil.

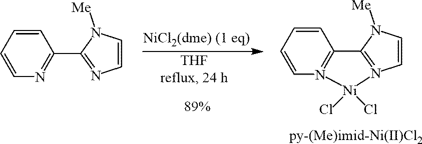

To a stirred solution of 2—(Me—imidazol-2-yl)pyridine (5.85 g, 36.3 mmol) in dry THF (Baker; 160 mL, 0.2 M) was added NiCl₂•DME (Stream; 7.98 g, 36.3 mmol) under inert atmosphere in glove box. The heterogeneous mixture was stirred at reflux for 24 hours out of glove box. The resulting mixture was cooled to room temperature and filtered through a glass filter funnel to collect the green solid. The resulting green solid was washed with Et₂O. ground to fine powder, and dried under reduced pressure using drying pistol technique at 140° C. (xylenes) for 15 hours to afford py-(Me)imid·Ni(II)Cl₂ complex (9.27 g, 32.3 mmol, 89%).

### Py-(Me)imid-NiBr2

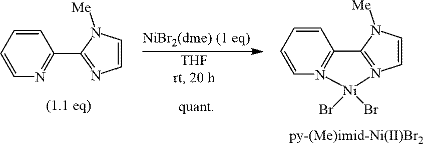

To a stirred solution of 2-(Me-imidazol-2-yl)pyridine (546 mg, 3.38 mmol) in dry THF (Baker; 15.4 mL, 0.2 M) was added NiBr₂•DME (Stream; 950 mg, 3.07 mmol) under inert atmosphere in glove box. The heterogeneous mixture was stirred at room temperature for 20 hours in glove box. The resulting mixture was filtered through a glass filter funnel to collect the yellow solid. The resulting solid was washed with Et₂O, ground to fine powder, and dried under reduced pressure using drying pistol technique at 140° C. (xylenes) for 15 hours to afford py-(Me)imid·Ni(II)Br₂ complex (1.15 g, 3.07 mmol, quant.).

### (Me)₃tpy·Ni(II)Cl₂

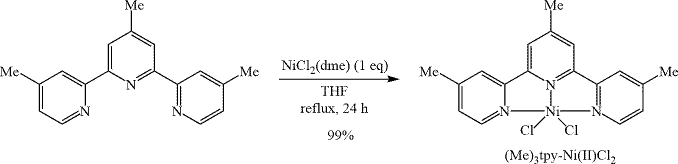

To a stirred solution of 4,4′,4″-trimethyl-2,2′:6′,2″-terpyridine (Stream; 2.06 g, 7.49 mmol) in dry THF (Baker; 34 mL, 0.2 M) was added NiCl₂•DME (Stream; 1.65 g, 7.49 mmol) under inert atmosphere in glove box. The heterogeneous mixture was stirred at reflux for 24 hours out of glove box. The resulting mixture was cooled to room temperature and filtered through a glass filter funnel to collect the blue solid. The resulting blue solid was washed with Et₂O, ground to fine powder, and dried under reduced pressure using drying pistol technique at 140° C. (xylenes) for 15 hours to afford (Me)3tpy·Ni(II)Cl₂ complex (3.00 g, 7.44 mmol, 99%).

### (dtbbpy)•NiCl₂

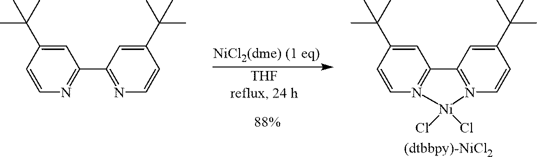

To a stirred solution of 4,4′-di-tert-butyl-2,2′-dipyridyl (Aldrich: 200 mg, 0.745 mmol) in dry THF (Baker; 3.4 mL, 0.2 M) was added NiCl₂•DME (Stream; 164 mg, 0.745 mmol) under inert atmosphere in glove box. The heterogeneous mixture was stirred at reflux for 24 hours out of glove box. The resulting mixture was cooled to room temperature and filtered through a glass filter funnel to collect the blue solid. The resulting blue solid was washed with Et₂O, ground to fine powder, and dried under reduced pressure using drying pistol technique at 140° C. (xylenes) for 15 hours to afford (dtbbpy)·NiCl₂ complex (260 mg, 0.653 mmol, 88%).

### (dtbbpy)·NiBr₂

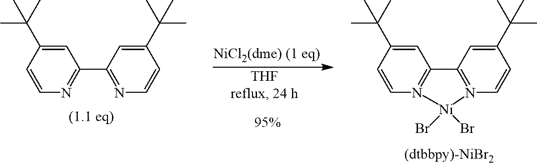

To a stirred solution of 4,4′-di-tert-butyl-2,2′-dipyridyl (Aldrich: 957 mg, 3.56 mmol) in dry THF (Baker; 16 mL, 0.2 M) was added NiBr₂•DME (Stream; 1.00 g, 3.24 mmol) under inert atmosphere in glove box. The heterogeneous mixture was stirred at room temperature for 24 hours in glove box.

The resulting mixture was cooled to room temperature and filtered through a glass filter funnel to collect the yellow solid. The resulting solid was washed with Et₂O, ground to fine powder, and dried under reduced pressure using drying pistol technique at 140° C. (xylenes) for 15 hours to afford (dtbbpy)·NiBr₂ complex (1.50 g, 3.08 mmol, 95%).

### (Me)₃tpy•Ni(I)I

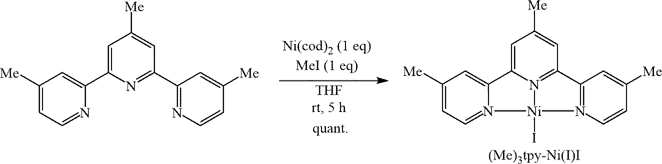

According to a procedure reported by Vicic¹⁵, to a stirred solution of 4,4′,4″-trimethyl-2,2′:6′,2″-terpyridine (Stream; 1.00 g, 3.63 mmol) in dry THF (Baker; 31 mL, 0.2 M) was added Ni(cod)₂ (Stream; 1.00 g, 3.63 mmol) under inert atmosphere in glove box. After stirring for 30 minutes, a solution of MeI (0.230 mL, 3.63 mmol) and THF (8 mL) was added slowly to the mixture. After further stirring for 5 hours, the mixture was diluted with dry pentane (Aldrich, 90 mL) out of glove box and stored in refrigerator overnight. The mixture was filtered through a glass filter funnel to collect the brown solid. The resulting solid was washed with hexane and Et₂O, ground to fine powder, and dried under reduced pressure using drying pistol technique at 140° C. (xylenes) for 15 hours to afford (Me)₃tpy-Ni(I)I complex (1.67 g, 3.63 mmol, quant.).

### Optimization Studies

The optimization of coupling condition was been started with the mixture of bidentate-ligand·NiX₂, (Me)₃tpy·Ni(II)Cl₂, and (Me)₃tpy-Ni(I)I. The tables below summarize the reaction optimization.

### Evaluation of Nickel Catalysts

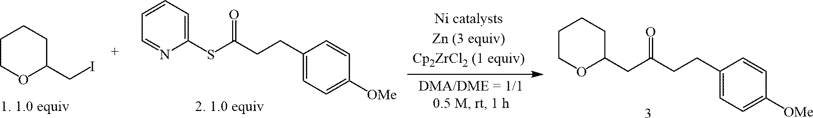

| entry | (dtbppy)·NiBr₂ (mol%) | py-(Me)imid·Ni(II)Cl₂ (mol%) | (Me)₃tpy·Ni(II)Cl₂ (mol%) | (Me)₃tpy·Ni(I)I (mol%) | yield′ (%) |
|---|---|---|---|---|---|
| 1 | 20 | none | none | none | 58 |
| 2 | 20 | none | 5 | none | 69 |
| 3 | none | 20 | none | none | 48 |
| 4 | none | 20 | 5 | none | 86 |
| 5 | none | 20 | none | 5 | 88 |

DMA = N,N-Dimethytacetemide DME = 1.2-Dimethoxyethane *Isolated yields.

### Evaluation of Substrate Ratios for 3

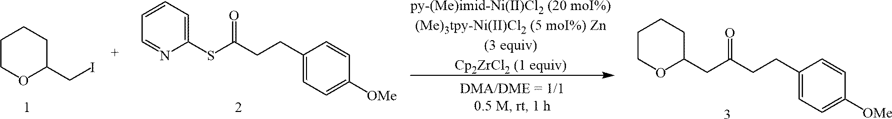

| entry | 1 (equiv) | 2 (equiv) | yield* (%) |
|---|---|---|---|
| 1 | 1.2 | 1.0 | 92 |
| 2 | 1.1 | 1.0 | 89 |
| 3 | 1.0 | 1.0 | 86 |
| 4 | 1.0 | 1.1 | 87 |
| 5 | 1.0 | 1.2 | 88 |

*Isolated yields.

### Evaluation of Solvents and Concentrations for 3

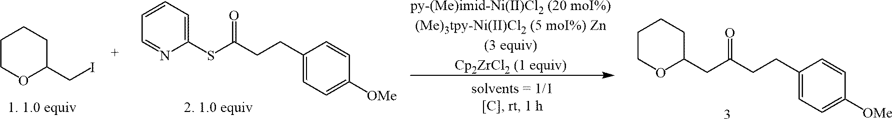

| entry | solvents (1/1) | concentration [C] | yield (%)* |
|---|---|---|---|
| 1 | DMF/DME | 0.5 M | 65 |
| 2 | DMI/DME | 0.5 M | 80 |
| 3 | DMA/THF | 0.5 M | 75 |
| 4 | DMA/1,4-dioxane | 0.5 M | 82 |
| 5 | DMA/DME DMA/ | 0.5 M | 86 |
| 6 | DME DMA/DME | 0.2 M | 82 |
| 7 | | 0.1 M | 77 |

DMF = N,N-Dimethyliomamide DMI = 1,8-Dimethyl-2-imidazolidinone Isolated yields.

### Evaluation of Catalytic Loadings

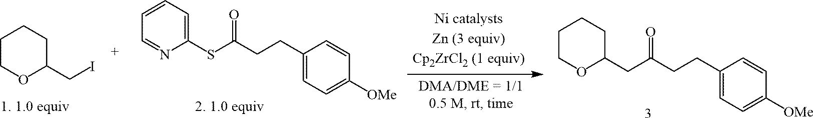

| entry | py-(Me)imid·Ni(II)Cl₂ (mol%) | (Me)₃tpy·Ni(II)Cl₂ (mol%) | (Me)₃tpy·Ni(II) (mol%) | time (h) | yield* (%) |
|---|---|---|---|---|---|
| 1 | 20 | 5 | none | 1 | 86 |
| 2 | 20 | none | 5 | 1 | 88 |
| 3 | 27 | none | 5 | 1 | 71 |
| 4 | 5 | none | 5 | 1 | 90 |
| 5 | 5 | none | 1 | 3 | 96 |
| 6 | 1 | none | 5 | 3 | 91 |
| 7 | 1 | none | 1 | 3 | 96 |
| 8 | 1 | 1 | none | 3 | 87 |
| 9 | 2 | none | none | 3 | 46 |
| 10 | none | none | 2 | 3 | 22 |

*Isolated yields.

From the above screens, it was found that (Me)₃tpy·Ni(I)I was better than (Me)₃tpy·Ni(H)Cl₂ in obtaining higher yields.

### Comparison of Zinc and Manganese as Reductant

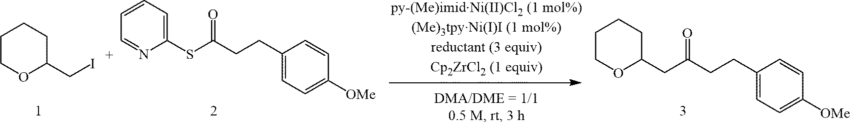

| entry | reductant | X (equiv) | Y (equiv) | yield* (%) |
|---|---|---|---|---|
| 1 | Zn | 1.1 | 1.0 | 96 |
| 2 | Zn | 1.0 | 1.0 | 96 |
| 3 | Zn | 1.0 | 1.1 | 94 |
| 4 | Mn | 1.1 | 1.0 | 85 |
| 5 | Mn | 1.0 | 1.0 | 85 |
| 6 | Mn | 1.0 | 1.1 | 84 |

*Isolated yleids.

### Relationship of Catalyst Loadings and Concentrations for S2

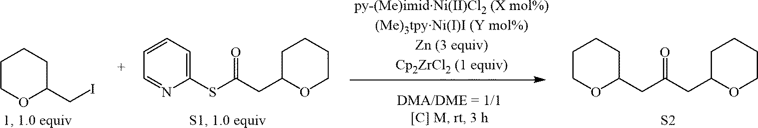

| entry | [C] M | X mol% | Y mol% | yield (%)* |
|---|---|---|---|---|
| 1 | 05 | 1 | 1 | 85 |
| 2 | 0.2 | 1 | 1 | poor conversion |
| 3 | 0.2 | 5 | 5 | 87 |
| 4 | 0.1 | 10 | 10 | 76 |
| 5 | 0.1 | 20 | 10 | 79 |
| 6 | 0.1 | 20 | 5 | 85 |
| 7 | 0.1 | 25 | 5 | 86 |

*Isolated yields.

### Evaluation of Substrate Ratios for S2

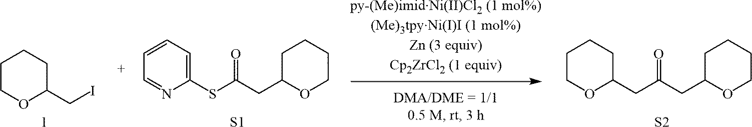

| entry | 1 (equiv) | 81 (equiv) | yield * (%) |
|---|---|---|---|
| 1 | 1.1 | 1.0 | 91 |
| 2 | 1.0 | 1.0 | 85 |
| 3 | 1.0 | 1.1 | 83 |

*Isolated yields.

### Evaluation of Bidentate Ligand·Ni(II) Complexes

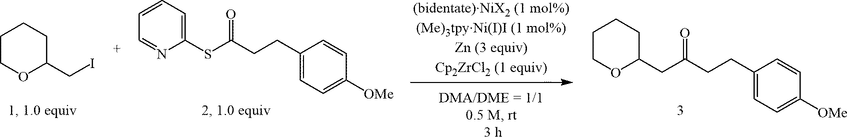

| entry | (bidentate)NiX₂ | yield (%)* |
|---|---|---|
| 1 | py-(Me)imid-Ni(II)Cl₂ | 96 |
| 2 | py-(Me)imid·Ni(II)Br₂ | 69 |
| 3 | (dtbbpy)·NiCl₂ | 79 |
| 4 | (dtbbpy)·NiBr₂ | 63 |

*Isolated yields.

### Evaluation of Terpyridine·Ni Complexes

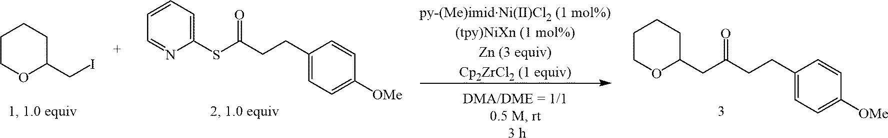

| | | |
|---|---|---|
| 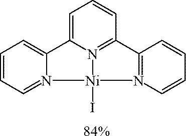 | 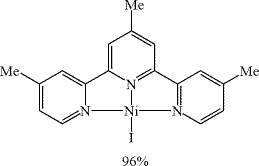 | 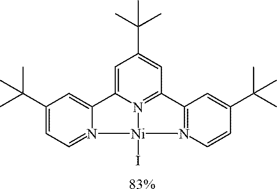 |
| 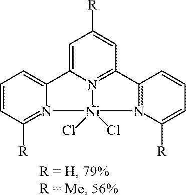 | 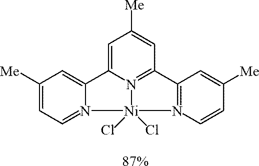 | 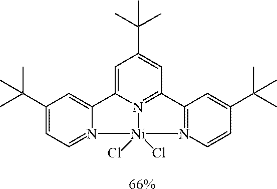 |

*Isolated yields.

### Evaluation of Additives

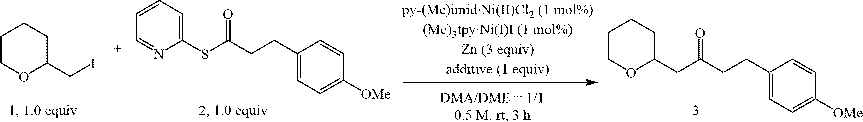

| entry | additive | yield (%) * |
|---|---|---|
| 1 | Cp₂ZrCl₂ | 96 |
| 2 | Cp₂TiCl₂ | complex mixture |
| 3^{b} | (Cp₂ZrCl)₂ | 62 |

^{a} Isolated yields. ^{b} THF was used instead of DME. Reaction was carried out for 8 h.

### Preparation of (Cy₂ZrCl)₂

According to a procedure reported by Schwartz¹⁶, to a stirred colorless solution of Cp₂ZrCl₂ (145 mg, 0.500 mmol) in dry THF (Baker; 0.5 mL) was added 20% Na(Hg) (60.0 mg, 0.525 mmol) under inert atmosphere in glove box and the mixture was stirred for 14 hours. The resulting deep red mixture was used for the ketone-coupling in entry 3 without further purification.

### Effects of Cp₂ZrCl₂ for 3

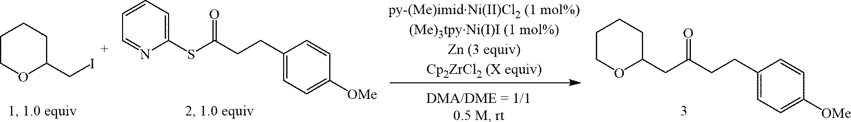

[image]

It was discovered that the Cp₂ZrCl₂ enhanced the coupling rate and decreased the productions of ester S3 and dimer 4.

### Procedure for the Reaction in Entry 5

To a solution of iodide 1 (37.3 mg, 0.165 mmol, 1.0 equiv) and thiopyridine ester 2 (45.0 mg, 0.165 mmol, 1.0 equiv) in DMA (0.17 mL) and DME (0.17 mL) were added py-(Me)imid·Ni(II)Cl₂ (0.50 mg, 1.65 µmol, 1 mol%) and (Me)₃tpy·Ni(I)I (0.80 mg, 1.65 µmol, 1 mol%) in the absence of Cp₂ZrCl₂. After stirring for 1 minute, Zn powder (32.4 mg, 0.495 mmol, 3.0 equiv) was added at room temperature. After being stirred at the same temperature for 6 hours, the reaction mixture was diluted with Et₂O and sat. NaHCO₃ aq. The organic layer was separated and the aqueous layer was extracted with Et₂O five times. The combined organic layer was washed with water three times and brine one time, dried over Na₂SO₄, filtered, and concentrated under reduced pressure. The obtained crude material was purified by flash column chromatography on silica gel to give ketone 3 (31.6 mg, 0.121 mmol, 73%), ester S3 (6.00 mg, 0.023 mmol, 14%), and dimer 4 (0.70 mg, 0.007 mmol, 4%).

### Effects of Cp₂ZrCl₂ with Different Acyl Derivatives

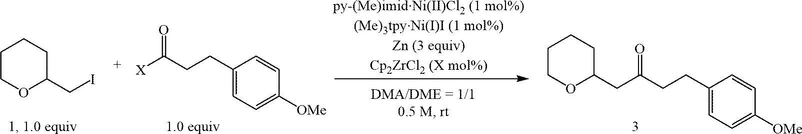

| entry | x = | time (h) | Cp₂ZrCl₂ (X mol%) | 3 (%) | S3 (%) | 4 (%) |
|---|---|---|---|---|---|---|
| 1 | 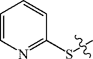 | 3 | 100 | 96 | 0 | 0 |
| 2 | | 6 | 0 | 73 | 14 | 4 |
| 3 | 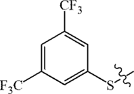 | 5 | 100 | 76 | 0 | 15 |
| 4 | | 5 | 0 | 40 | 28 | 32 |
| 5 | 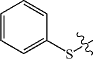 | 5 | 100 | 7 | 0 | 35 |
| 6 | | 5 | 0 | 7 | 10 | 43 |
| 7 | 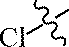 | 3 | 100 | 51 | 0 | 40 |
| 8 | | 3 | 0 | 28 | 11 | 57 |

*Isolated yields.

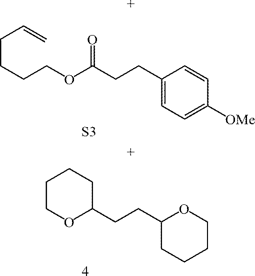

### Mechanistic Studies

### Hydrozirconation/Ketone Coupling

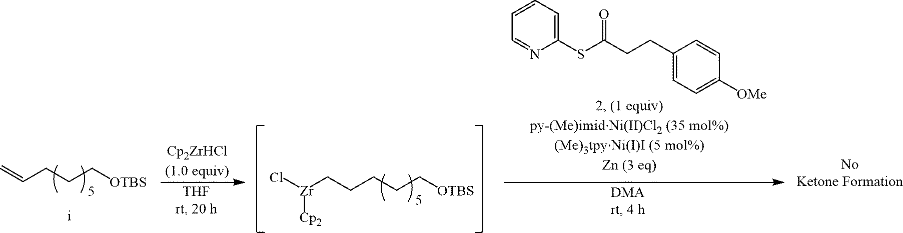

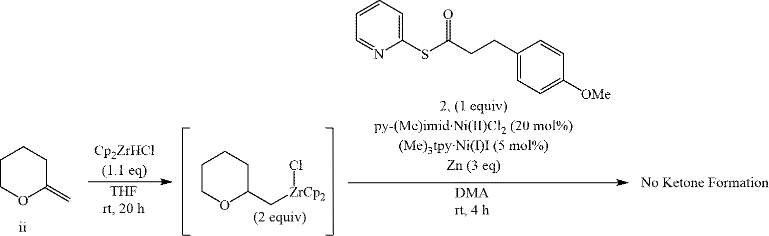

In a glove box, to a solution of olefin (i or ii) in THF was added Cp₂ZrHCl (1.0-1.1 equiv). After stirring for 20 hours, a mixture of thiopyridine ester 2, catalysts, and Zn in DMA (0.42 mL) were added. After being stirred at the same temperature for 4 hours, the reaction mixture was removed from glove box and diluted with Et₂O and sat. NaHCO₃ aq. (1 mL). The organic layer was separated and the aqueous layer was extracted with Et₂O five times. The combined organic layer was washed with water three times and brine one time, dried over Na₂SO₄, filtered, and concentrated under reduced pressure. The no ketone formations were detected from ¹H-NMR analysis of the crude materials.

### Consumption Rate of Thiopyridine Ester 2

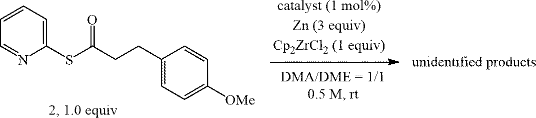

| entry | catalyst | time (h) | recovered yield¹ of 2 (%) |
|---|---|---|---|
| 1 | none | 14 | 97 |
| 2 | (dtbbpy)·NiBr₂ | 0.5 | 93 |
| 3 | | 3 | 85 |
| 4 | | 14 | 72 |
| 5 | py-(Me)imid·Ni(II)Cl₂ | 0.5 | 89 |
| 6 | | 3 | 64 |
| 7 | | 14 | 11 |
| 8 ² | | 14 | 79 |
| 9 ³ | | 14 | 87 |
| 10 | (Me)₃tpy·(II)I | 0.5 | 99 |
| 11 | | 3 | 96 |
| 12 | | 14 | 78 |
| 13 ₄ | py-(Me)imid·Ni(COD)₂ | 0.5 | 0 |
| 14 ₄ | dtbby·Ni(COD)₂ | 0.5 | 0 |
| 15 ₄ | (Me)₃ipy·Ni(COD)₂ | 0.5 | 0 |

¹ *Isolated yields. ² Without Cp₂ZrCl₂³ Without Zn. ⁴ 1 Equivalent of ligand·Ni(COD)₂ was used without Zn. The reactions were carried out at 0.2 M.

### Coupling of Bromide

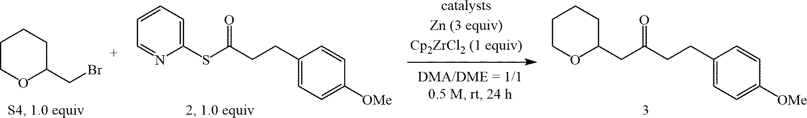

| entry | py-(Me)imid·Ni(II)Cl₂ (mol%) | (dtbbpy·NiBr₂ (mol%) | (Me)₃tpy·Ni(II)I (mol%) | yield(%)^{*} |
|---|---|---|---|---|
| 1 | 2 | none | none | <1 |
| 2 | none | 2 | none | <1 |
| 3 | none | none | 2 | 9 |
| 4 | 1 | none | 1 | 24 |
| 5 | 1 | none | 5 | 28 |

^{*}Isolated yields.

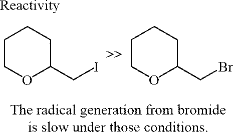

### β-Elimination and Dimerization of Bromide

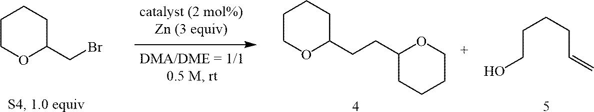

| entry | catalyst | time (h) | bromide | 4 ¹ (%) | 5 ² (%) |
|---|---|---|---|---|---|
| 1 | none | 18 | no conversion | 0 | 0 |
| 2 | py-(Me)imid·Ni(II)Cl₂ | 9 | poor conversion | <1 | <1 |
| 3 | (dtbbpy)·NiBr₂ | | poor conversion | <1 | <1 |
| 4 | (Me)₃tpy·Ni(II)I | 9 9 | consumed poor | 19 | detected |
| 5 ³ | py-(Me)imid·Ni(COD)₂ | 1 | conversion poor | <1 | <1 |
| 6 ³ | dtbbpy·Ni(COD)₂ | 1 | conversion | <1 | <1 |
| 7 ³ | (Me)₃tpy·Ni(COD)₂ | 1 | consumed | 33 | detedcted |

¹ Isolated yields. ² Not *Isolated ³ 1 Equivalent of ligand·Ni(COD)ₐ was used without Zn. The reactions were carried out at 0.2 M.

### β-Elimination and Dimerization of Iodide

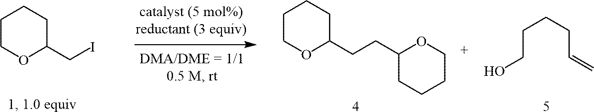

| entry | reductant | catalyst (mol%) | time (h) | 1, lodide (%) | 4 ¹ (%) | 5 ² (%) |
|---|---|---|---|---|---|---|
| 1 | Zn | none | 2 | 0 | 0 | only detectable |
| 2 | Zn | (Me)₃tpy·Ni(I)I | 2 | 0 | 20 | ∼80 |
| 3³ | Zn | (Me)₃tpy·Ni(I)I | 2 | 0 | 72 | ∼28 |
| 4 | Mn | none | 4 | no conversion | 0 | 0 |
| 5 | Mn | (Me)₃tpy·M(I)I | 14 | 0 | 66 | ∼34 |
| 6³ | none | (Me)₃tpy·Ni(I)I | 8 | no conversion | 0 | 0 |
| 7³ | none | (Me)₃tpy·Ni(COD)₂ | 1.5 | 0 | 55 | ∼45 |

¹ *Isolated yield. ²Not Isolated. ³ 1 Equivalent of ligand·Ni was used. Reactions were carried out at 0.2 M.

### Stoichiometric Ni(COD)₂ mediated-Coupling

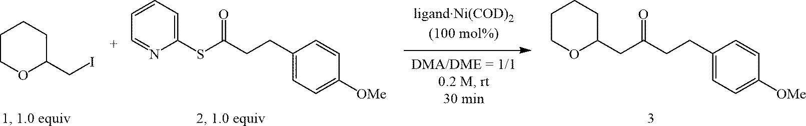

| entry | ligand | 1 ¹ (%) | 2 ¹ (%) | 3 ² (%) | 4 ² (%) |
|---|---|---|---|---|---|
| 1 | py-(Me)imid | 40 | 0 | 55 | <1 |
| 2 | dtbppy | 50 | 0 | 46 | <1 |
| 3 | (Me)₃tpy | 0 | 18 | 26 | 40 |

¹Recovered yields. ²isolated yields.

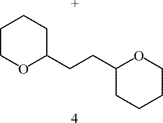

In a glove box, to a solution of ligand (1.0 equiv) in DMA (0.42 mL) was added Ni(COD)₂ (45.1 mg, 0.165 mmol, 1.0 equiv). After stirring for 30 minutes, a mixture of iodide 1 (37.3 mg, 0.165 mmol, 1.0 equiv) and thiopyridine ester 2 (45.0 mg, 0.165 mmol, 1.0 equiv) in DME (0.42 mL) were added. After being stirred at the same temperature for 30 minutes, the reaction mixture was removed from glove box and diluted with Et₂O and sat. NaHCO₃ aq. (1 mL). The organic layer was separated and the aqueous layer was extracted with Et₂O five times. The combined organic layer was washed with water three times and brine one time, dried over Na₂SO₄, filtered, and concentrated under reduced pressure. The obtained crude material was purified by flash column chromatography on silica gel to give products and starting materials.

### Evaluation of Negishi-Coupling Pathway

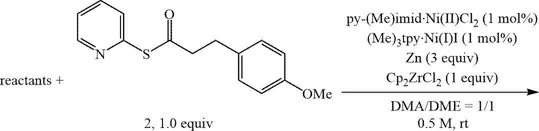

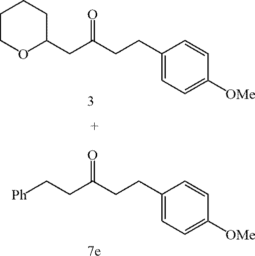

| entry | reactants | time (n) | 3* (%) | 7e* (%) |
|---|---|---|---|---|
| 1 | 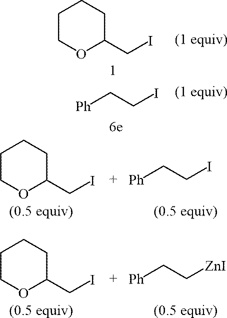 | 3 | 96 | 0 |
| 2 | | 3 | 0 | 92 |
| 3 | | 1.5 | 84 | 100 |
| 4 | | 1.5 | 98 | 58 |

*isolated yields based on reactants.

### Experimental Evidence of a Radical Pathway

### Radical Ring-Opening/Ketone Coupling

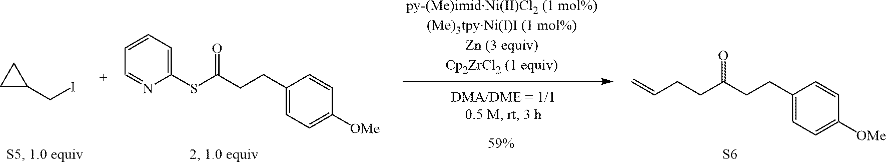

In a glove box, to a solution of iodomethylcyclopropane S5 (30.0 mg, 0.165 mmol, 1.0 equiv) and thiopyridine ester 2 (45.0 mg, 0.165 mmol, 1.0 equiv) in DMA (0.17 mL) and DME (0.17 mL) were added py-(Me)imid·Ni(II)Cl₂ (0.50 mg, 1.65 µmol, 1 mol%), (Me)₃tpy·Ni(I)I (0.80 mg, 1.65 µmol, 1 mol%), and Cp₂ZrCl₂ (48.2 mg, 0.165 mmol, 1.0 equiv). After stirring for 1 minute, Zn powder (32.4 mg, 0.495 mmol, 3.0 equiv) was added at room temperature. After being stirred at the same temperature for 3 hours, the reaction mixture was removed from glove box and diluted with Et₂O (1 mL) and sat. NaHCO₃ aq. (1 mL). The organic layer was separated and the aqueous layer was extracted with Et₂O (1 mL) five times. The combined organic layer was washed with water (1 mL) three times and brine (1 mL) one time, dried over Na₂SO₄, filtered, and concentrated under reduced pressure. The obtained crude material was purified by flash column chromatography on silica gel to give S6 (21.4 mg, 0.098 mmol, 59%) as colorless oil.

### Radical 5-exo-trig Cyclization/Ketone Coupling¹⁷

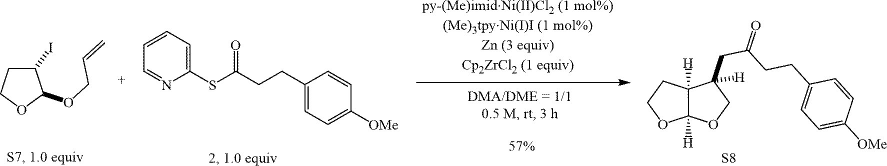

In a glove box, to a solution of iodide S7 (41.9 mg, 0.165 mmol, 1.0 equiv) and thiopyridine ester 2 (45.0 mg, 0.165 mmol, 1.0 equiv) in DMA (0.17 mL) and DME (0.17 mL) were added py-(Me)imid·Ni(II)Cl₂ (0.50 mg, 1.65 µmol, 1 mol%), (Me)₃tpy·Ni(l)I (0.80 mg, 1.65 µmol, 1 mol%), and Cp₂ZrCl₂ (48.2 mg, 0.165 mmol, 1.0 equiv). After stirring for 1 minute, Zn powder (32.4 mg, 0.495 mmol, 3.0 equiv) was added at room temperature. After being stirred at the same temperature for 3 hours, the reaction mixture was removed from glove box and diluted with Et₂O (1 mL) and sat. NaHCO₃ aq. (1 mL). The organic layer was separated and the aqueous layer was extracted with Et₂O (1 mL) five times. The combined organic layer was washed with water (1 mL) three times and brine (1 mL) one time, dried over Na₂SO₄, filtered, and concentrated under reduced pressure. The obtained crude material was purified by flash column chromatography on silica gel to give S8 (27.1 mg, 0.923 mmol, 57%) as colorless oil.

### Ketone Coupling in the Halichondrin Synthesis

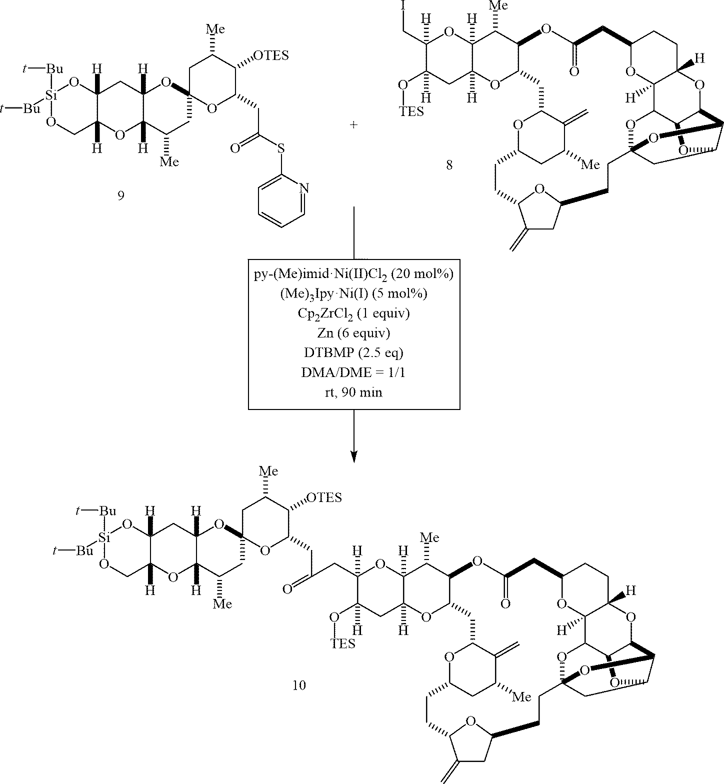

### General Procedure for 3

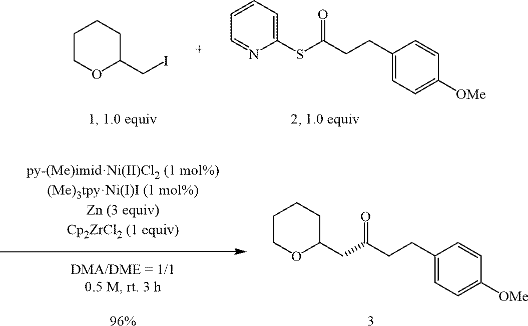

In a glove box, to a solution of iodide 1 (37.3 mg, 0.165 mmol, 1.00 equiv) and thiopyridine ester 2 (45.0 mg, 0.165 mmol, 1.00 equiv) in DMA (0.17 mL) and DME (0.17 mL) were added py-(Me)imid-Ni(II)Cl₂ (0.50 mg, 1.65 µmol, 1 mol%), (Me)₃tpy·Ni(I)I (0.80 mg, 1.65 µmol, 1 mol%), and Cp₂ZrCl₂ (48.2 mg, 0.165 mmol, 1.0 equiv). After stirring for 1 minute, Zn powder (32.4 mg, 0.495 mmol, 3.0 equiv Sigma-aldrich, used without any activation) was added at room temperature. After being stirred at the same temperature for 3 hours, (monitored by TLC), the reaction mixture was removed from glove box and diluted with Et₂O (1 mL) and sat. NaHCO₃ aq. (1 mL). The organic layer was separated and the aqueous layer was extracted with Et₂O (1 mL) five times. The combined organic layer was washed with water (1 mL) three times and brine (1 mL) one time, dried over Na₂SO₄, filtered, and concentrated under reduced pressure. The obtained crude material was purified by flash column chromatography (hexanes/AcOEt = 5:1) on silica gel to afford ketone 3 (41.6 mg, 0.159 mmol, 96°fo) as colorless oil.

### Final Ketone Coupling in the Halichondrin Synthesis

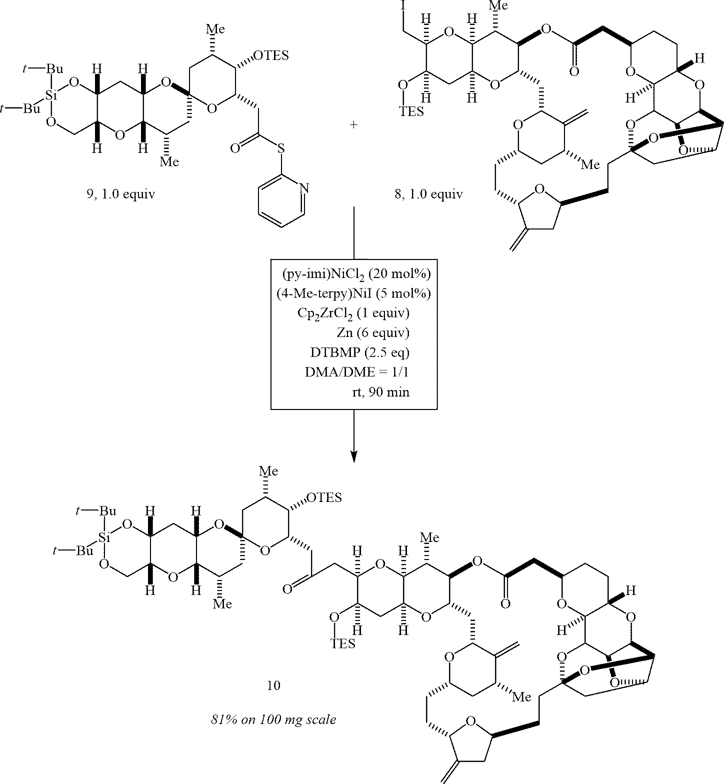

In a glove box, to a solution of iodide 8 (100 mg, 0.101 mmol, 1.00 equiv), thiopyridine ester 9 (72.0 µg, 0.101 µmol, 1.00 equiv), and DTBMP (51.9 mg, 0.253 mmol, 2.5 equiv) in DMA (0.26 mL) and DME (0.26 mL) were added py-(Me)imid·Ni(II)Cla (5.8 µg, 0.0202 µmol, 20 mol%), (Me)₃tpy·Ni(I)I (2.3 µg, 0.00505 µmol, 5 mol%), and Cp₂ZrCl₂ (29.5 mg, 0.101 mmol, 1 equiv) at room temperature. After stirring for 1 minute, Zn (39.6 mg, 0.606 mmol, 6.0 equiv Sigma-aldrich, used without any activation) was added to a mixture. After further stirring for 90 minutes, the reaction was quenched with a mixture of saturated aqueous NaHCO₃ and water (1/1), and the resulting mixture was extracted with EtOAc five times. The combined organic extracts were dried over anhydrous Na₂SO₄, filtered, and concentrated under reduced pressure. The residue was purified by flash silica gel column chromatography using YAMAZEN (hexanes/AcOEt = 3:1) to afford ketone 10 (119 mg, 0.0818 mmol, 81%) as a colorless amorphous solid.

### Characterization Data

### 2-Methyl-1H-imidazol-2-yl)pyridine

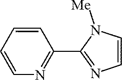 [SMILES: Cn1ccnc1-c1ccccn1]

IR (film) 3105, 1588, 1490, 1462, 1278, 1034, 790, 742, 707 cm⁻¹; ¹H NMR (500 MHz, CDCl₃) δ = 8.66-8.49 (m, 1H), 8.18 (dt, J = 8.1, 1.2 Hz, 1H), 7.75 (ddd, J = 9.5, 7.1, 1.7 Hz, 1H), 7.21 (ddd, J = 7.6, 4.9, 1.3 Hz, 1H), 7.12 (d, J = 1.4 Hz, 1H), 6.97 (s, 1H), 4.13 (d, J= 1.2 Hz, 3H); ¹³C NMR (126 MHz, CDCl₃) δ = 150.79, 148.19, 144.96, 136.55, 128.18, 124.37, 122.64, 122.33, 36.28; HRMS (ESI) m/z calc. for C₉H₁₀O₃ [M+H]+ 160.0875; found 160.0864.

### 4-Methoxyphenyl)-1-(tetrahydro-2H-pyran-2-yl)butan-2-one, 3

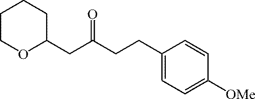 [SMILES: COc1ccc(CCC(=O)CC2CCCCO2)cc1]

3 was synthesized according to the general procedure. The crude product was purified by flash silica gel column chromatography (hexanes:EtOAc = 5:1) to afford 3 (41.6 mg, 0.159 mmol, 96%) as a colorless oil. IR (film) 2934, 2849, 1712, 1612, 1513, 1441, 1300. 1246, 1178, 1087, 1043, 828 cm⁻¹; ¹H NMR (500 MHz, CDCl₃) δ = 7.09 (d, J= 8.4 Hz, 2H), 6.81 (d, J = 8.4 Hz, 2H), 3.91 (d,J=11.4 Hz, 1H), 3.78 (s, 3H), 3.77-3.72 (m, 1H), 3.41 (t, J =1 0.8 Hz, 1 H), 2.83 (t,J=7.8 Hz, 2H), 2.74 (q, J = 5.4 Hz, 2H), 2.64 (dd, J =15.6 Hz, 7.8 Hz, 1H), 2.36 (dd, J = 15.6 Hz, 5.2 Hz, 1H), 1.80 (d, J = 5.2 Hz, 1H), 1.58 (d, J=12.6 Hz, 1H), 1.53-1.46 (m, 3H), 1.29-1.21 (m, 1H); ¹³C NMR (126 MHz, CDCl₃) δ = 210.2, 158.1, 135.7, 133.9, 133.3, 129.8, 129.3, 127.8, 114.0, 63.1, 55.4, 44.7, 39.5, 29.1, 27.0, 26.7, 19.3; HRMS (ESI) m/z calc. for C₁₆H₂₃O₃ [M+H]+ 263.1642; found 263.1649.

### 1,2-Bis(tetrahydro-2H-pyran-2-yl)ethane, 4

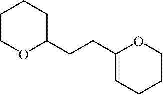 [SMILES: C1CCC(CCC2CCCCO2)OC1]

Colorless oil. IR (film) 2930, 2838, 1086, 1047, 897 cm⁻¹; ¹H NMR (500 MHz, CDCl₃) δ = 4.14-3.86 (m, 2H), 3.42 (td, J = 11.6, 2.3 Hz, 2H), 3.32-3.08 (m, 2H), 1.82 (dt, J = 13.2, 2.9 Hz, 2H), 1.54 (dddd, J= 49.2, 16.8, 7.2, 3.7 Hz, 11H), 1.34-1.15 (m, 3H); ¹³C NMR (126 MHz, CDCl₃) δ = 78.14, 77.26, 68.47, 32.91, 32.10, 31.99, 31.76, 26.23, 23.60, 23.58; HRMS (ESI) m/z calc. for C₁₂H₂₃O₂ [M+H]⁺ 199.1698; found 199.1687.

### 1-(2,2-Dimethyl-1,3-dioxolan-4-yl)-4-(4-methoxyphenyl)butan-2-one, 7a

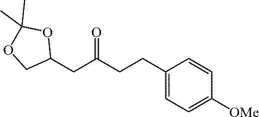 [SMILES: COc1ccc(CCC(=O)CC2COC(C)(C)O2)cc1]

7a was synthesized according to the general procedure. The crude product was purified by flash silica gel column chromatography (hexanes:EtOAc = 5:1) to afford 7a (44.0 mg, 0.158 mmol, 96%) as a colorless oil. IR (film) 3035, 2988, 2935, 1711, 1612, 1513, 1478, 1370, 1246, 1178, 1058, 1036, 829, 669 cm⁻¹; ¹H NMR (500 MHz, CDCl₃) δ = 7.09 (d, J= 8.5 Hz, 2H), 6.81 (d, J= 8.5 Hz, 2H), 4.44 (quin, J= 6.0 H, 1H), 4.15 (dd, J= 8.5 Hz, 8.0 Hz, 1H), 3.77 (s, 3H), 3.50 (dd, J = 8.5 Hz, 8.0 Hz, 1H), 2.88-2.80 (m, 3H), 2.76-2.71 (m, 2H), 2.52 (dd, J= 16.5 Hz, 7.0 Hz, 1H) 1.38 (s, 3H), 1.33 (s, 3H); ¹³C NMR (126 MHz, CDCl₃) δ = 207.8, 158.0, 132.8, 129.2, 113.9, 108.8, 71.7, 69.4, 55.2, 47.2, 45.2, 28.7, 26.9, 25.5; HRMS (ESI) m/z calc. for C₁₆H₂₂NaO₄ [M+Na]+ 301.1410; found 301.1425.

### tert-Butyl(S)-2-(4-(4-methoxyphenyl)-2-oxobutyl)pyrrolidine-1-carboxylate, 7b

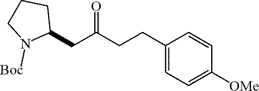 [SMILES: COc1ccc(CCC(=O)C[C@@H]2CCCN2C(=O)OC(C)(C)C)cc1]

7b was synthesized according to the general procedure. The crude product was purified by flash silica gel column chromatography (hexanes:EtOAc = 4:1) to afford 7b (51.6 mg, 0.149 mmol, 90%) as a colorless oil.

$${\left[\text{α}\right]}_{\text{D}}^{22} = - 58.0 \left(c 2.0 , {\text{CHCl}}_{3}\right) ;$$

IR (film) 2971, 1685, 1512, 1391, 1364, 1243, 1166, 1107, 1034, 827, 772 cm⁻¹; ¹H NMR (500 MHz, CDCl₃) δ = 7.10 (d,J= 8.5 Hz, 2H), 6.82 (d, J= 8.6 Hz, 2H), 4.14 (d, J = 10.3 Hz, 1H), 3.79 (s, 3H), 3.32 (d, J= 6.6 Hz, 2H), 2.78 (dt, J = 60.4, 7.6 Hz, 2H), 2.69 (dd, J = 16.1, 9.7 Hz, 2H), 2.37 (dd, J = 16.1, 9.7 Hz, 1H), 2.03 (dq, J = 12.7, 8.1 Hz, 1H), 1.78 (ddd, J= 9.1, 6.4, 3.9 Hz, 2H), 1.59 (dtd, J = 12.6, 5.6, 3.3 Hz, 1H), 1.45 (s, 10H); ¹³C NMR (126 MHz, CDCl₃) δ = 208.71, 157.94, 154.27, 133.03, 129.23, 113.87, 79.33, 55.25, 53.43, 47.34, 46.33, 44.97, 31.09, 28.83, 28.53, 23.28; HRMS (ESI) m/z calc. for C₂₀H₃₀NO₄ [M+H]⁺ 348.2175; found 348.2240.

### tert-Butyl(RH5-(4-methoxyphenyl)-3-oxo-1-phenylpentyl)carbamate, 7c

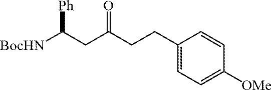 [SMILES: COc1ccc(CCC(=O)C[C@@H](NC(=O)OC(C)(C)C)c2ccccc2)cc1]

7c was synthesized according to the general procedure. The crude product was purified by flash silica gel column chromatography (hexanes:EtOAc = 5:1) to afford 7c (60.5 mg, 0.158 mmol, 96%) as a white solid.

$${\left[\text{α}\right]}_{\text{D}}^{22} = 14.7$$

[a]t2= 14.7 (c 0.3, CHCl₃); IR (film) 3376, 2979, 2932, 1707, 1612, 1513, 1455, 1366, 1247, 1175, 1037, 819, 701 cm⁻¹; ¹H NMR (500 MHz, CDCl₃) δ = 7.33-7.29 (m, 2H), 7.27-7.22 (m, 3H), 7.00 (d, J= 8.4 Hz, 2H), 6.78 (d, J= 8.4 Hz, 2H), 5.46 (brs, 1H), 5.07 (brs, 1H), 3.78 (s, 3H), 3.00 (brs, 1H), 2.85 (dd, J = 17.4 Hz, 4.3 Hz, 1H), 2.73 (t, J =7.8 Hz, 2H), 2.69-2.62 (m, 1H), 2.59-2.52 (m, 1H), 1.41 (s, 9H); ¹³C NMR (126 MHz, CDCl₃) δ = 208.5, 158.1, 155.3, 141.7, 132.9, 129.3, 128.8, 127.5, 126.4, 114.0, 79.9, 55.4, 51.3, 48.8, 45.4, 28.7, 28.5; HRMS (ESI) m/z calc. for C₂₃H₂₉NNaO₄ [M+Na]⁺ 406.1989; found 406.1980.

### tert-Butyl(S)-(6-(4-methoxyphenyl)-oxo-1-phenylhexan-2-yl)carbamate, 7d

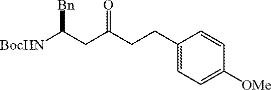 [SMILES: COc1ccc(CCC(=O)C[C@H](Cc2ccccc2)NC(=O)OC(C)(C)C)cc1]

7d was synthesized according to the general procedure. The crude product was purified by flash silica gel column chromatography (hexanes: EtOAc = 5.1) to afford 7d (60.9 mg, 0.153 mmol, 93%) as a white solid.

$${\left[\text{α}\right]}_{\text{D}}^{22} = - 5.7$$

-5.7 (c 1.1, CHCl₃); IR (film) 3360, 2977, 2931, 1708, 1612, 1513, 1455, 1391, 1366, 1301, 1247, 1174, 1109, 1077, 1037, 824, 778, 702 cm⁻¹; ¹H NMR (500 MHz, CDCl₃) δ = 7.29-7.25 (m, 2H), 7.21 (t, J= 7.2 Hz, 1H), 7.10 (d, J = 7.2 Hz, 2H), 7.08 (d,J= 9.0 Hz, 2H), 6.82 (d, J = 9.0 Hz, 2H), 5.04 (brs, 1H), 4.11 (brs, 1H), 3.78 (s, 3H), 2.91 (brs, 1H), 2.84-2.75 (m, 3H), 2.71-2.58 (m, 2H), 2.54 (d, J =4.9 Hz, 2H), 1.40 (s, 9H); ¹³C NMR (126 MHz, CDCl₃) δ = 209.5, 158.1, 155.4, 138.2, 132.9, 129.4, 129.3, 128.7, 126.7, 114.1, 79.5, 55.4, 48.9, 45.6, 45.1, 40.4, 28.8, 28.5; HRMS (ESI) m/z calc. for C₂₄H₃₂NO₄ [M+H]⁺ 398.2331; found 398.2326.

### 1-Methoxyphenyl)-5-phenylpentan-3-one, 7e

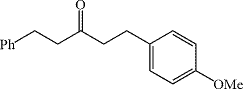 [SMILES: COc1ccc(CCC(=O)CCc2ccccc2)cc1]

7e was synthesized according to the general procedure. The crude product was purified by flash silica gel column chromatography (hexanes:EtOAc = 10:1) to afford 7e (40.9 mg, 0.152 mmol, 92%) as a colorless oil. IR (film) 3061, 3027, 2932, 2835, 1712, 1611, 1512, 1247, 1035, 823 cm⁻¹; ¹H NMR (500 MHz, CDCl₃) δ = 7.35-7.25 (m, 2H), 7.25-7.14 (m, 3H), 7.10 (dd, J = 8.4, 1.5 Hz, 2H), 6.91-6.74 (m, 2H), 3.80 (s, 3H), 2.88 (dt,J = 25.9, 7.6 Hz, 4H), 2.79-2.55 (m, 4H); ¹³C NMR (126 MHz,CDCl₃) δ = 209.26, 157.97, 141.04, 133.03, 129.24, 128.49, 128.31, 126.10, 113.91, 55.26, 44.78, 44.55, 29.73, 28.91; HRMS (ESI) m/z calc. for C₁₈H₂₁O₂ [M+H]⁺ 269.1542; found 269.1503.

### 1-(1H-Indol-3-yl)-5-(4-methoxyphenyl)pentan-3-one, 7f

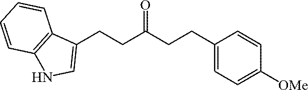 [SMILES: COc1ccc(CCC(=O)CCc2c[nH]c3ccccc23)cc1]

7f was synthesized according to the general procedure. The crude product was purified by flash silica gel column chromatography (hexanes:EtOAc = 4:1) to afford 7f (45.6 mg, 0.148 mmol, 90%) as a white solid. IR (film) 3409, 2930, 1706, 1511, 1456, 1244, 1178, 1092, 1033, 822, 743 cm⁻¹; ¹H NMR (500 MHz, CDCl₃) δ = 7.98 (br, 1H), 7.57 (dq, J= 7.9, 1.0 Hz, 1H), 7.35 (dt, J= 8.2, 0.9 Hz, 1H), 7.21 (ddd, J= 8.1, 7.0, 1.3 Hz, 1H), 7.13 (ddd, J= 8.0, 7.0, 1.1 Hz, 1H), 7.10-7.00 (m, 2H), 6.94 (dd, J= 2.3, 1.1 Hz, 1H), 6.86-6.67 (m, 2H), 3.79 (s, 3H), 3.05 (ddd, J= 7.6, 6.9, 0.9 Hz, 2H), 2.82 (dt, J= 20.0, 7.4 Hz, 4H), 2.69 (dd, J= 8.1, 7.0 Hz, 2H); ¹³C NMR (126 MHz, CDCl₃) δ = 210.13, 157.91, 136.27, 133.09, 129.25, 127.14, 122.03, 121.52, 119.29, 118.68, 115.16, 113.86, 111.16, 55.27, 44.74, 43.46, 28.93, 19.33; HRMS (ESI) m/z calc. for C₂₀H₂₂NO₂ [M+H]⁺ 308.1651; found 308.1620.

### 1-Methoxyphenyl)-5,5-dimethylhexan-3-one, 7 g

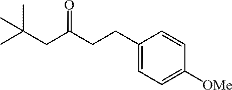 [SMILES: COc1ccc(CCC(=O)CC(C)(C)C)cc1]

7 g was synthesized according to the general procedure. The crude product was purified by flash silica gel column chromatography (hexanes:EtOAc =10:1) to afford 7 g (34.8 mg, 0.149 mmol, 90%) as a colorless oil. IR (film) 2953, 1708, 1512, 1363, 1244, 1177, 1035, 824 cm⁻¹; ¹H NMR (500 MHz, CDCl₃) δ = 7.09 (d, J= 8.6 Hz, 2H), 6.82 (d, J= 8.6 Hz, 2H), 3.78 (s, 3H), 2.81 (t, J = 7.6 Hz, 2H), 2.74-2.57 (m, 2H), 2.28 (s, 2H), 0.99 (s, 9H); ¹³C NMR (126 MHz, CDCl₃) δ = 210.17, 157.95, 133.35, 129.34, 113.90, 55.33, 55.28, 46.98, 31.13, 29.81, 28.90; HRMS (ESI) m/z calc. for C₁₅H₂₃O₂ [M+H]⁺ 235.1698; found 235.1959.

### 1-Cyclohexyl-3(4-methoxyphenyl)propan-1-one, 7h

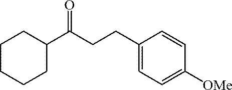 [SMILES: COc1ccc(CCC(=O)C2CCCCC2)cc1]

7h was synthesized according to the general procedure. The crude product was purified by flash silica gel column chromatography (hexanes:EtOAc = 10:1) to afford 7h (36.6 mg, 0.149 mmol, 90%) as a colorless oil. IR (film) 2927, 2852, 1703, 1511, 1243, 1176, 1034, 822 cm⁻¹; ¹H NMR (500 MHz, CDCl₃) δ = 7.11 (d,J= 8.6 Hz, 2H), 6.83 (dd,J= 8.5, 1.3 Hz, 2H), 3.80 (d, J = 1.2 Hz, 3H), 2.84 (t, J = 7.5 Hz, 2H), 2.79-2.62 (m, 2H), 2.44-2.14 (m, 1H), 1.94-1.72 (m, 4H), 1.67 (dp, J= 11.8, 4.1, 2.7 Hz, 1H), 1.50-1.01 (m, 5H); ¹³C NMR (126 MHz, CDCl₃) δ = 213.27, 157.89, 133.47, 129.24, 113.84, 55.25, 50.99, 42.50, 28.87, 28.40, 25.86, 25.66; HRMS (ESI) m/z calc. for C₁₆H₂₃O₂ [M+H]⁺ 247.1698; found 247.1744.

### 4-(4-Methoxyphenyl)-3-oxopentyl)phenyl-trifluoromethanesulfonate, 7i

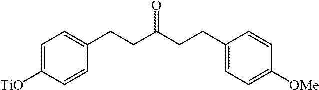

7i was synthesized according to the general procedure. The crude product was purified by flash silica gel column chromatography (hexanes:EtOAc = 5:1) to afford 7i (57.8 mg, 0.139 mmol, 84%) as a colorless oil. IR (film) 1713, 1512, 1417, 1247, 1208, 1137, 885, 728, 608 cm⁻¹; ¹H NMR (500 MHz, CDCl₃) δ = 7.26-7.20 (m, 2H), 7.21-7.14 (m, 2H), 7.13-6.99 (m, 2H), 6.91-6.70 (m, 2H), 3.80 (s, 3H), 2.92 (t, J= 7.4 Hz, 2H), 2.85 (t, J= 7.5 Hz, 2H), 2.71 (td, J= 7.4, 4.9 Hz, 4H), ¹³C NMR (126 MHz, CDCl₃) δ = 208.55, 158.01, 147.90, 141.73, 132.81, 130.14, 129.23, 121.26, 120.01, 117.46, 113.92, 113.88, 55.24, 44.71, 44.06, 28.90, 28.81; HRMS (ESI) m/z calc. for C₁₉H₂₀F₃NaO₅S [M+H]⁺ 439.0803; found 439.0832.

### 1-Bromophenyl)-5-methoxyphenyl)pentan-3-one, 7j

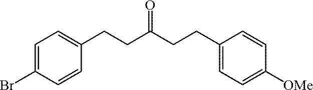 [SMILES: COc1ccc(CCC(=O)CCc2ccc(Br)cc2)cc1]

7j was synthesized according to the general procedure. The crude product was purified by flash silica gel column chromatography (hexanes:EtOAc = 10:1) to afford 7j (47.3 mg, 0.136 mmol, 83%) as a colorless oil. IR (film) 2931, 1711, 1511, 1487, 1243, 1177, 1034, 1010, 814, 516 cm⁻¹; ¹H NMR (500 MHz, CDCl₃) δ = 7.45-7.31 (m, 2H), 7.06 (dd, J = 19.4, 8.1 Hz, 4H), 6.83 (d, J = 8.3 Hz, 2H), 3.80 (d, J = 1.2 Hz, 3H), 2.84 (t. J = 7.5 Hz, 4H), 2.68 (t, J = 7.5 Hz, 4H); ¹³C NMR (126 MHz, CDCl₃) δ = 208.83, 157.98, 140.04, 132.89, 131.50, 130.13, 129.23, 119.84, 113.91, 55.27, 44.76, 44.21, 29.01, 28.89; HRMS (ESI) m/z calc. for C₁₈H₂₀BrO₂ [M+H]⁺ 347.0647; found 347.0601.

### 1-Lodophenyl)-5-methoxyphenyl)pentan-3-one, 7k

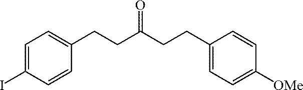 [SMILES: COc1ccc(CCC(=O)CCc2ccc(I)cc2)cc1]

7k was synthesized according to the general procedure. The crude product was purified by flash silica gel column chromatography (hexanes:EtOAc = 10:1) to afford 7k (40.0 mg, 0.101 mmol, 61%) as a colorless oil. IR (film) 2929, 1710, 1510, 1242, 1176, 1033, 1005, 809, 513 cm⁻¹; ¹H NMR (500 MHz, CDCl₃) δ = 7.59 (d, J= 7.9 Hz, 2H), 7.15 - 7.00 (m, 2H), 6.92 (d, J = 8.0 Hz, 2H), 6.83 (d, J = 8.3 Hz, 2H), 3.80 (s, 3H), 2.84 (td, J= 7.6, 4.4 Hz, 4H), 2.68 (t,J= 7.4 Hz, 4H); ¹³C NMR (126 MHz, CDCl₃) δ = 208.82, 157.98, 140.72, 137.49, 132.89, 130.48, 129.23, 113.91, 91.15, 55.28, 44.76, 44.18, 29.11, 28.89; HRMS (ESI) m/z calc. for C₁₈H₂₀IO₂ [M+H]⁺ 395.0508; found 395.0535.

### 1-Hydroxyphenyl)-5-methoxyphenyl)pentan-3-one, 71

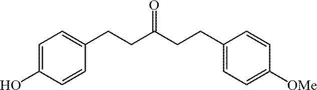 [SMILES: COc1ccc(CCC(=O)CCc2ccc(O)cc2)cc1]

71 was synthesized according to the general procedure. The crude product was purified by flash silica gel column chromatography (hexanes:EtOAc = 10:1) to afford 71 (39.8 mg, 0.139 mmol, 85%) as a colorless oil. IR (film) 3380, 2931, 1701, 1611, 1511, 1442, 1243, 1177, 1033, 825, 542 cm⁻¹; ¹H NMR (500 MHz, CDCl₃) δ = 7.08 (d,J= 8.6 Hz, 2H), 7.02 (d,J= 8.4 Hz, 2H), 6.83 (d,J= 8.5 Hz, 2H), 6.75 (d, J= 8.4 Hz, 2H), 5.30 (br, 1H), 3.80 (s, 3H), 2.83 (q, J= 7.1 Hz, 4H), 2.69 (td, J= 7.5, 3.0 Hz, 4H); ¹³C NMR (126 MHz, CDCl₃) δ = 210.20, 157.91, 154.02, 133.00, 132.90, 129.41, 129.25, 115.34, 113.93, 55.30, 44.82, 28.90 (Two signals are missing due to overlap); HRMS (ESI) m/z calc. for C₁₈H₂₀NaO₃ [M+Na]⁺ 307.1310; found 307.1320.

### Ketone, 10

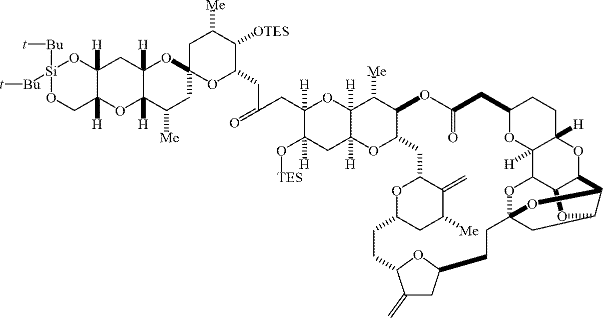

All analytical data for 10 was in accordance with our previous literature¹⁸.

### 1,3-Bis(tetrahydro-2H-pyran-2-yl)propan-2-one, S2

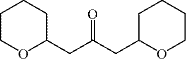 [SMILES: O=C(CC1CCCCO1)CC1CCCCO1]

S2 was synthesized according to the general procedure. The crude product was purified by flash silica gel column chromatography (hexanes:EtOAc = 4:1) to afford S2 (31.7 mg, 0.140 mmol, 85%) as a colorless oil. IR (film) 2933, 2487, 1713, 1440, 1378, 1356, 1203, 1175, 1088 cm⁻¹; ¹M NMR (500 MHz, CDCl₃) δ = 3.91 (d, J= 9.2 Hz, 2H), 3.79-3.73 (m, 2H), 3.43 (dd, J= 11.2, 10.8 Hz, 2H), 2.67 (dd, J= 14.8, 5.8 Hz, 2H), 2.44 (dd, J= 14.8, 5.8 Hz, 2H), 1.80 (d, J= 7.2 Hz, 2H), 1.62-1.58 (m, 3H), 1.52-1.46 (m, 5H), 1.30-1.21 (m, 2H), ¹³C NMR (126 MHz, CDCl₃) δ = 207.7, 74.1, 68.7, 50.6, 50.4, 31.9, 25.9, 23.5; HRMS (ESI) m/z calc. for C₁₃H₂₂NaO₃ [M+Na]⁺ 249.1467; found 249.1460.

### Hexen-1-yl 3-(4-methoxyphenyl)propanoate, S3

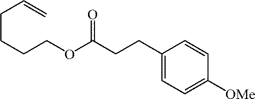 [SMILES: C=CCCCCOC(=O)CCc1ccc(OC)cc1]

Colorless oil. IR (film) 2935, 1729, 1512, 1244, 1175, 1034, 911, 824 cm⁻¹; ¹H NMR (500 MHz, CDCl₃) δ= 7.14 (dd, J= 8.8, 0.7 Hz, 2H), 6.84 (d, J= 8.5 Hz, 2H), 5.80 (ddt, J= 16.9, 10.2, 6.7 Hz, 1H), 5.15-4.88 (m, 2H), 4.08 (t, J = 6.6 Hz, 2H), 3.80 (d, J= 0.6 Hz, 3H), 2.91 (t, J= 7.8 Hz, 2H), 2.61 (dd, J= 8.2, 7.3 Hz, 2H), 2.20-1.94 (m, 2H), 1.73-1.55 (m, 2H), 1.43 (tt,J= 9.9, 6.5 Hz, 2H); ¹³C NMR (126 MHz, CDCl₃) δ = 173.04, 158.05, 138.34, 132.61, 129.22, 114.80, 113.87, 64.35, 55.23, 36.22, 33.27, 30.16, 28.06, 25.17; HRMS (ESI) m/z calc. for C₁₆H₂₃O₃ [M+H]⁺ 263.1647; found 263.1683.

### 1-Methoxyphenyl)hept-6-en-3-one, S6

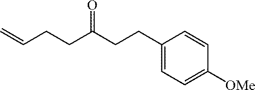 [SMILES: C=CCCC(=O)CCc1ccc(OC)cc1]

S6 was synthesized according to the general procedure. The crude product was purified by flash silica gel column chromatography (hexanes:EtOAc = 20:1 ) to afford S6 (21.4 mg, 0.098 mmol, 59%) as a colorless oil. IR (film) 2926, 1753, 1612, 1513, 1442, 1365,1301, 1246, 1178, 1109, 1036, 911, 829 cm⁻¹; ¹H NMR (500 MHz, CDCl₃) δ = 7.09 (d, J= 8.5 Hz, 2H), 6.82 (d, J = 8.5 Hz, 2H), 5.83-5.73 (m, 1H), 5.01 (dd, J= 17.5 Hz, 1.4 Hz, 1H), 4.97 (dd, J= 10.0 Hz, 1.4 Hz, 1H), 3.78 (s, 3H), 2.84 (t,J=7.5 Hz, 2H), 2.70 (t,J= 7.5 Hz, 2H), 2.48 (t, J=7.5 Hz, 2H), 2.31 (q, J=7.5 Hz, 2H); ¹³C NMR (126 MHz, CDCl₃) δ = 209.4, 158.0, 137.1, 133.1, 129.2, 115.2, 114.0, 55.3, 44.6, 42.0, 28.9, 27.7; HRMS (ESI) m/z calc. for C₁₄H₁₉O₂ [M+H]⁺ 219.1380; found 219.1374.

### 1-(Hexahydrofuro[2,3-b]furan-3-yl)-4-(4-methoxyphenyl)butan-2-one, S8

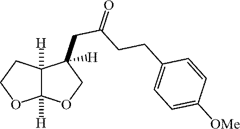 [SMILES: COc1ccc(CCC(=O)C[C@H]2CO[C@H]3OCC[C@@H]23)cc1]

S8 was synthesized according to the general procedure. The crude product was purified by flash silica gel column chromatography (hexanes:EtOAc = 2:1) to afford S8 (27.1 mg, 0.093 mmol, 57%) as a colorless oil. IR (film) 2951, 1710, 1611, 1512, 1244, 1030, 1001, 828 cm⁻¹; ¹H NMR (500 MHz, CDCl₃) δ = 7.10 (d, J= 8.2 Hz, 2H), 6.94-6.73 (m, 2H), 5.83-5.59 (m, 1H), 3.99 (t, J= 7.9 Hz, 1H), 3.91-3.69 (m, 5H), 3.45-3.27 (m, 1H), 2.97-2.80 (m, 3H), 2.79-2.64 (m, 3H), 2.55 (dd, J = 17.6, 7.6 Hz, 1H), 2.45 (dd, J= 17.6, 7.0 Hz, 1H), 1.90-1.72 (m, 1H), 1.66 (dq, J= 12.2, 5.9 Hz, 1H); ¹³C NMR (126 MHz, CDCl₃) δ = 208.10, 158.06, 132.70, 129.23, 113.95, 109.47, 71.69, 68.91, 55.27, 45.08, 44.64, 41.13, 36.89, 28.96, 25.48; HRMS (ESI) m/z calc. for C ₁₇H₂₃O₄ [M+H]⁺ 291.1596; found 291.1584.

### REFERENCES

1) For Ni-mediated one-pot ketone syntheses, see:
   a) Mukaiyama group: M. Onaka, Y. Matsuoka, T. Mukaiyama, Chem. Lett. 1981, 10, 531.
   b) Weix group: A. C. Wotal, D. J. Weix, Org. Lett. 2012, 14, 1476; A. C. Wotal, R. D. Ribson, D. J. Weix, Organometallics 2014, 33, 5874; D. J. Weix, Acc. Chem. Res. 2015, 48, 1767.
   c) Gong group: H. Yin, C. Zhao, H. You, K. Lin, H. Gong, Chem. Commun. 2012, 48, 7034; C. Zhao, X. Jia, X. Wang, H. Gong, J. Am. Chem. Soc. 2014, 136, 17645.
   d) Reisman group: A. H. Cherney, N. T. Kadunce, S. E. Reisman, J. Am. Chem. Soc. 2013, 135, 7442.
   e) Baran group: S. Ni, N. M. Padial, C. Kingston, J. C. Vantourout, D. C. Schmitt, J. T. Edwards, M. M. Kruszyk, R. R. Merchant, P. K. Mykhailiuk, B. B. Sanchez, S. Yang, M. A. Perry, G. M. Gallego, J. J. Mousseau, M. R. Collins, R. J. Cherney, P. S. Lebed, J. S. Chen, T. Qin, P. S. Baran, J. Am. Chem. Soc. 2019, 141, 6726. Link, CAS
2)
   a) Y. Ai, N. Ye, Q. Wang, K. Yahata, Y. Kishi, Angew. Chem., Int. Ed. 2017, 56, 10791.
   b) K. Yahata, N. Ye, Y. Ai, K. Iso, Y. Kishi, Angew. Chem., Int. Ed. 2017, 56, 10796. Crossref, Medline, CAS
3) A. Umehara, K. Umihara, Y. Kishi, unpublished results.
4)
   a) T. J. Anderson, G. D. Jones, D. A. Vicic, J. Am. Chem. Soc. 2004, 126, 8100,
   b) G. D. Jones, C. McFarland, T. J. Anderson, D. A. Vicic, Chem. (Commun. 2005, 4211.
   c) G. D. Jones, J. L. Martin, C. McFarland, O. R. Allen, R. E. Hall, A. D. Haley, R. J. Brandon, T. Konovalova, P. J. Desrochers, P. Pulay, D. A. Vicic, J. Am. Chem. Soc. 2006, 128, 13175.
   d) J. T. Ciszewski, D. Y. Mikhaylov, K. V. Holin, M. K. Kadirov, Y. H. Budnikova, O. Sinyashin, D. A. Vicic, Inorg. Chem. 2011, 50, 8630.
   e) D. Mikhaylov, T. Gryaznova, Y. Dudkina, M. Khrizanphorov, S. Latypov, O. Kataeva, D. A. Vicic, O. G. Sinyashin, Y. Budnikova, Dalton Trans. 2012, 41, 165. Crossref, Medline, CAS
5) The coupling smoothly proceeded in a 1:1 mixture of DMA-DME, DMA-dioxane, and DMA-THF. DMA can be replaced with 1,3-dimethylimidazolidin-2-one (DMI) and DMA.
6) For the details, see Supporting Information.
7) Experiments with commonly-used radical probes support an involvement of a radical(s) in the ketone coupling, cf. i → ii and iii → iv.

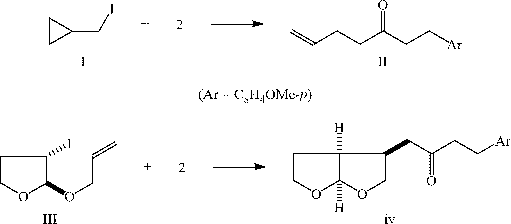

8) For selected papers and reviews on Ni^{I}, see for example:
   a) X. Lin, D. L. Phillips, J. Org. Chem. 2008, 73, 3680.
   b) K. M. Arendt, A. G. Doyle, Angew. Chem., Int. Ed. 2015, 54, 9876.
   c) C.-Y. Lin, P. P. Power, Chem. Soc. Rev. 2017, 46, 5347.
   d) Y. H. Budnikova, D. A. Vicic, A. Klein, Inorganics 2018, 6, 18. Crossref, Medline, CAS
9) For example, see: M. T. Quirós, D. Collado-Sanz, E. Buñuel, D. J. Cárdenas, J. Phys. Chem. A 2018, 122, 2250. Crossref, Medline, CAS
10) Experimentally, this was shown from the following experiment; 2 was first treated with py-(Me)imid·Ni^{II}Cl₂ (1 mol %), Cp₂Zr^{IV}C₂ (1 equiv), Zn (3 equiv) in 1:1 DMA/DME at RT for 1 hour. Then, 1 (1 equiv) and (Me)₃tpy-Ni^{I}I (1 mol %) in DMA/DME were added, and 3 was isolated in a yield comparable to the standard condition.
11)
   a) G. M. Williams, K. I. Gell, J. Schwartz, J. Am. Chem. Soc. 1980, 102, 3660.
   b) G. M. Williams, J. Schwartz, J. Am. Chem. Soc. 1982, 104, 1122.
   c) K. Fujita, T. Nakamura, H. Yorimitsu, K. Oshima, J. Am. Chem. Soc. 2001, 123, 3137.
   For Cp₂Zr^{III}Cl and its dimeric form (prepared by reduction of Cp₂Zr^{IV}Cl₂ with Na/Hg), see: d) M. C. Barden, J. Schwartz, J. Org. Chem. 1997, 62, 7520. However, to our best knowledge, Cp₂Zr^{IV}Cl₂/Zn or Mn system is unknown. Crossref, Medline, CAS
12) At an early stage of study, we were interested in an alternative possibility: Cp₂Zr^{III}Cl could have a high reactivity towards R°, to form Cp₂Zr^{IV}(R)Cl, which might be involved in the ketone coupling. However, the experiment using hydrozirconation-products showed this possibility unlikely.
13) Experimentally, this was shown from the following experiment; 1was first treated with (Me)₃tpy•Ni^{I}I (1 mol %), Cp₂Zr^{IV}Cl₂ (1 equiv), Zn (3 equiv) in 1:1 DMA/DME at RT for 30 minutes. Then, 2 (1 equiv) and py-(Me)imid·Ni^{II}Cl₂ (1 mol %) in DMA/DME were added, but no ketone formation was observed.
14) The thiopyridine ester 2 was used for the study. Knowing that the activation of 2 is fast (see the text), we chose Ni^{I}/Ni^{II} = 1 mol %/1 mol %.
15) T. J. Anderson, G. D. Jones, D. A. Vicic, J. Am. Chem. Soc. 2004, 126, 8100.
16) M. C. Barden, J. Schwartz, J. Org. Chem. 1997, 62, 7520.
17) F. Wu, W. Lu, Q. Qian, Q. Ren, H. Gong, Org. Lett. 2012, 14, 3044.
18) K. Yahata, N. Ye, Y. Ai, K. Iso, Y. Kishi, Angew. Chem. Int. Ed. 2017, 56, 10796.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method for preparing a compound of Formula (C): 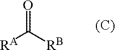
or a salt thereof, the method comprising coupling a compound of Formula (A): 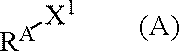
or a salt thereof, with a compound of Formula (B): 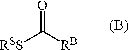
or a salt thereof, in the presence of a nickel(I) complex, a nickel(II) complex, and a zirconium complex; wherein:
X¹ is halogen or a leaving group;
R^{S} is optionally substituted alkyl, optionally substituted carbocyclyl, optionally substituted aryl, optionally substituted heterocyclyl, or optionally substituted heteroaryl;
R^{A} is optionally substituted alkyl; and
R^{B} is optionally substituted alkyl, optionally substituted alkenyl, optionally substituted alkynyl, optionally substituted aryl, optionally substituted carbocyclyl, optionally substituted heteroaryl, or optionally substituted heterocyclyl;
optionally wherein R^{A} and R^{B} are joined together via a linker, wherein the linker is selected from the group consisting of optionally substituted alkylene, optionally substituted heteroalkylene, optionally substituted alkenylene, optionally substituted heteroalkenylene, optionally substituted alkynylene, optionally substituted heteroalkynylene, optionally substituted arylene, optionally substituted heteroarylene, optionally substituted carbocyclylene, optionally substituted heterocyclylene, optionally substituted acylene, and combinations thereof.

2. The method of claim 1, wherein the compound of Formula (A) is of Formula (A-1): 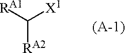
or a salt thereof; the compound of Formula (B) is of Formula (B-1): 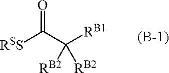
or a salt thereof; and the compound of Formula (C) is of Formula (C-1): 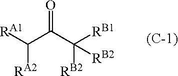
or a salt thereof, wherein:
R^{S} is optionally substituted alkyl, optionally substituted carbocyclyl, optionally substituted aryl, optionally substituted heterocyclyl, or optionally substituted heteroaryl;
each instance of R^{A1}, R^{A2}, R^{B1}, and R^{B2} is independently hydrogen, optionally substituted alkyl, optionally substituted alkenyl, optionally substituted alkynyl, optionally substituted aryl, optionally substituted carbocyclyl, optionally substituted heteroaryl, or optionally substituted heterocyclyl; optionally wherein R^{A1} and R^{B1} are joined together via a linker.

3. The method of claim 2 comprising reacting a compound of Formula (A-B): 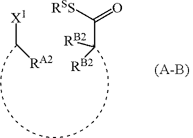
or a salt thereof, to prepare a compound of Formula (C-2): 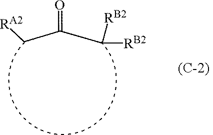
or salt thereof; wherein: 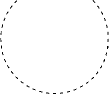
represents a linker.

4. The method of claim 1 or 2, wherein the compound of Formula (B) is of Formula (L-2-6): 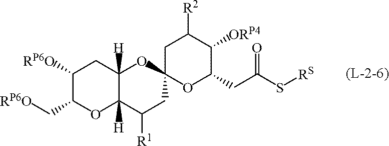
or a salt or stereoisomer thereof;
the compound of Formula (A) is of Formula (R-2-I): 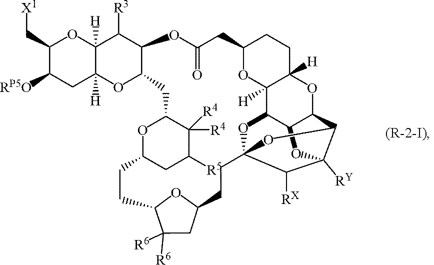
or a salt or stereoisomer thereof; and the compound of Formula (C) is of Formula (H3-2-II): 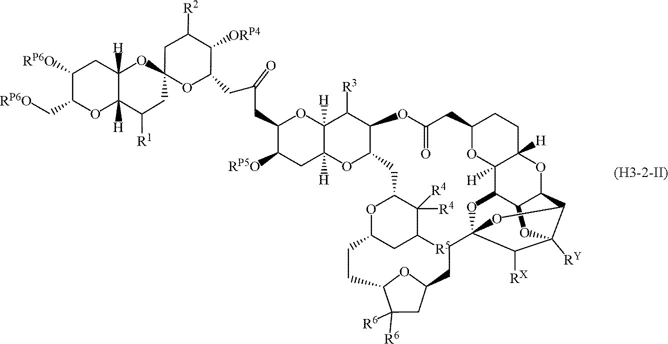
or a salt or stereoisomer thereof, wherein:
R¹, R², R³, and R⁵ are each independently hydrogen, halogen, or optionally substituted alkyl;
each instance of R⁴ is independently hydrogen, halogen, or optionally substituted alkyl, or two R⁴ groups are taken together to form: 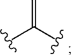
each instance of R⁶ is independently hydrogen, halogen, or optionally substituted alkyl, or two R⁶ groups are taken together to form: 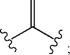
R^{P4}, R^{P5}, and R^{P6} are each independently hydrogen, optionally substituted alkyl, optionally substituted acyl, or an oxygen protecting group; optionally wherein two R^{P6} are joined with the intervening atoms to form optionally substituted heterocyclyl;
R^{X} is hydrogen or -OR^{Xa}, wherein R^{Xa} is hydrogen, optionally substituted alkyl, optionally substituted acyl, or an oxygen protecting group; and
R^{Y} is hydrogen or -OR^{Ya}, wherein R^{Ya} is hydrogen, optionally substituted alkyl, optionally substituted acyl, or an oxygen protecting group;
optionally wherein R^{Xa} and R^{Ya} are joined together with their intervening atoms to form optionally substituted heterocyclyl.

5. The method of claim 4, wherein the compound of Formula (B) is of Formula (E-L): 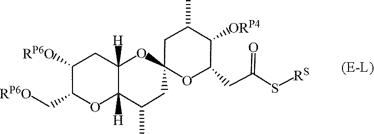
or a salt or stereoisomer thereof;
the compound of Formula (A) is of Formula (E-R): 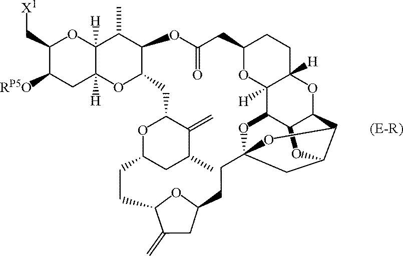
or a salt or stereoisomer thereof; and
the compound of Formula (C) is of Formula (E-1): 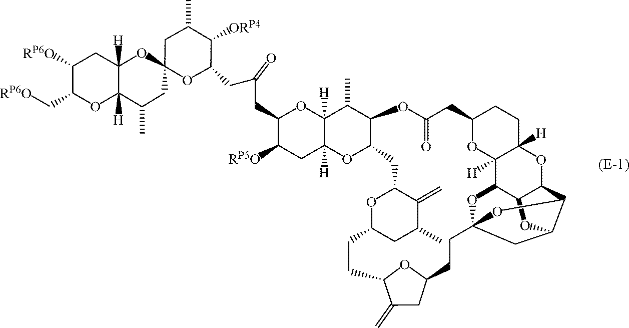
or a salt or stereoisomer thereof.

6. The method of claim 5, wherein the compound of Formula (B) is the following: 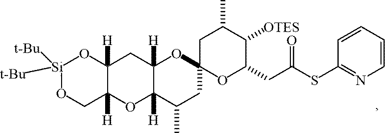
or a salt or stereoisomer thereof;
the compound of Formula (A) is the following: 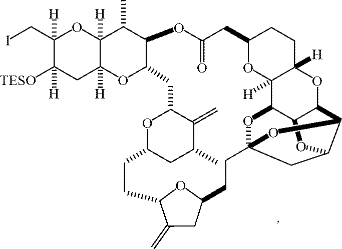
or a salt or stereoisomer thereof; and
the compound of Formula (C) is the following: 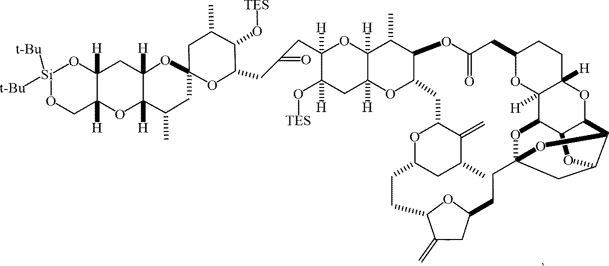
or a salt or stereoisomer thereof.

7. The method of any one of claims 1-6, wherein the nickel(I) complex is of the formula: NiX•(ligand); wherein X is halogen, and “ligand” is a tridentate ligand.

8. The method of claim 7, wherein the nickel(I) complex is used after being formed by complexation of a nickel source and the “ligand” in solution.

9. The method of claim 8, wherein the nickel source is of the formula: NiX₂.

10. The method of claim 8 or 9, wherein the nickel source is NiI₂.

11. The method of any one of claims 1-10, wherein the nickel(I) complex is of the formula: 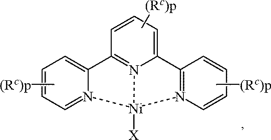
wherein:
X is a halogen;
each instance of p is independently 0 or an integer from 1-4, inclusive;
each instance of R^{c} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1};
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or two R^{N} bonded to the same nitrogen atom are taken together with the intervening atoms to form optionally substituted heterocyclyl or optionally substituted heteroaryl;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{S1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

12. The method of any one of claims 1-11, wherein the nickel(I) complex is of the formula: 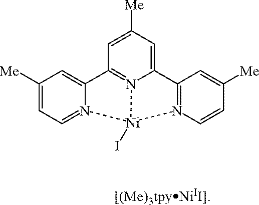
.

13. The method of any one of claims 1-12, wherein the nickel(I) complex is present in a catalytic amount.

14. The method of claim 13, wherein the nickel(I) complex is present in from about 0.1-10 mol% with respect to the compound of Formula (A) or the compound of Formula (B); preferably wherein the nickel(I) complex is present in about 1 mol% with respect to the compound of Formula (A) or the compound of Formula (B).

15. The method of claim 13, wherein the nickel(I) complex is present in from about 1-30 mol% the compound of Formula (A) or the compound of Formula (B).

16. The method of claim 13, wherein the nickel(I) complex is present in about 20 mol% with respect to the compound of Formula (A) or the compound of Formula (B).

17. The method of any one of claims 1-16, wherein the nickel(II) complex is of the formula: NiX₂•(ligand); wherein X is halogen, and “ligand” is a bidentate ligand.

18. The method of claim 17, wherein the nickel(II) complex is used after being formed by complexation of a nickel source and the “ligand” in solution.

19. The method of claim 18, wherein the nickel source is of the formula: NiX₂.

20. The method of claim 18 or 19, wherein the nickel source is NiCl₂.

21. The method of any one of claims 1-20, wherein the nickel(II) complex is of the formula: 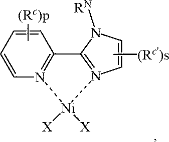
wherein:
each instance of X is a halogen;
p is 0 or an integer from 1-4, inclusive;
s is 0, 1, or 2;
each instance of R^{c} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1};
each instance of R^{c′} is independently hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —N(R^{N})₂, -OR^{O}, or -SR^{S1};
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or two R^{N} bonded to the same nitrogen atom are taken together with the intervening atoms to form optionally substituted heterocyclyl or optionally substituted heteroaryl;
each instance of R^{O} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group; and
each instance of R^{S1} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

22. The method of any one of claims 1-21, wherein the nickel(II) complex is of the formula: 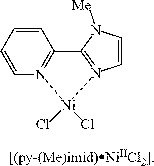
.

23. The method of any one of claims 1-22, wherein the nickel(II) complex is present in a catalytic amount.

24. The method of claim 23, wherein the nickel(II) complex is present in from about 0.1-10 mol% with respect to the compound of Formula (A) or the compound of Formula (B).

25. The method of claim 23, wherein the nickel(II) complex is present in from about 1 mol% with respect to the compound of Formula (A) or the compound of Formula (B).

26. The method of claim 23, wherein the nickel(II) complex is present in from about 1-20 mol% with respect to the compound of Formula (A) or the compound of Formula (B).

27. The method of claim 23, wherein the nickel(II) complex is present in about 5 mol% with respect to the compound of Formula (A) or the compound of Formula (B).

28. The method of any one of claims 1-27, wherein the zirconium complex is Cp₂ZrCl₂.

29. The method of any one of claims 1-28, wherein the zirconium complex is present in stoichiometric or excess amount.

30. The method of claim 29, wherein the zirconium complex is present in about 1 to about 4 equivalents with respect to the compound of Formula (A) or the compound of Formula (B).

31. The method of claim 29, wherein the zirconium complex is present in about 1 equivalent with respect to the compound of Formula (A) or the compound of Formula (B).

32. The method of any one of claims 1-31, wherein the step of coupling is carried out in the presence of zinc or manganese.

33. The method of claim 32 or 33, wherein the zinc or manganese is present in a stoichiometric or excess amount.

34. The method of claim 33, wherein the zinc or manganese is present in about 1-10 equivalents with respect to the compound of Formula (A) or the compound of Formula (B).

35. The method of claim 33, wherein the zinc or manganese is present in about 6 equivalents with respect to the compound of Formula (A) or the compound of Formula (B).

36. The method of any one of claims 32-35, wherein the reaction is carried out in the presence of zinc metal.

37. The method of any one of claims 1-36, wherein the step of coupling is carried out in the presence of a base or proton scavenger.

38. The method of claim 37, wherein the base or proton scavenger is di-tert-butylmethylpyridine (dtbmpy).

39. The method of any one of claims 1-38, wherein the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; and zinc or manganese metal.

40. The method of any one of claims 1-38, wherein the reaction is carried out in the presence of the nickel (I) complex: (Me)₃tpy•Ni^{I}I; the nickel(II) complex: (py-(Me)imid)•Ni^{II}Cl₂; the zirconium complex: Cp₂ZrCl₂; zinc metal; and 2,6-di-tert-butyl-4-methylpyridine.

41. The method of any one of claims 1-40, wherein the step of coupling is carried out in a solvent.

42. The method of claim 41, wherein the solvent comprises one or more of N,N-dimethylacetamide (DMA), 1,2-dimethoxyethane (DME), and 1,3-dimethyl-2-imidazolidinone (DMI).

43. The method of claim 41, wherein the solvent comprises a DMA/DME mixture.

44. The method of claim 41, wherein the solvent comprises a DMI/DME mixture.

45. The method of any one of claims 1-44, wherein the step of coupling is carried out at or around room temperature.

46. The method of any one of claims 1-45, wherein the compound of Formula (A) is present in in a range from about 1 equivalent to less than 1.3 equivalents with respect to the compound of Formula (B).

47. The method of any one of claims 1-46, wherein the compound of Formula (A) and the compound of Formula (B) are present in approximately 1:1 ratio.

48. The method of any one of claims 1-5 and 7-47, wherein X¹ is a halogen.

49. The method of any one of claims 1-5 and 7-48, wherein X¹ is —I.

50. The method of any one of claims 1-5 and 7-49, wherein R^{S} is optionally substituted heteroaryl.

51. The method of any one of claims 1-5 and 7-50, wherein R^{S} is optionally substituted pyridyl.

52. The method of any one of claims 1-5 and 7-51, wherein R^{S} is optionally substituted 2-pyridyl.

53. The method of any one of claims 1-5 and 7-52, wherein R^{S} is of the formula: 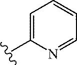
.

54. The method of any one of claims 4 and 7-53, wherein R¹ is optionally substituted C₁₋₆ alkyl.

55. The method of any one of claims 4 and 7-54, wherein R¹ is unsubstituted C₁₋₆ alkyl.

56. The method of any one of claims 4 and 7-55, wherein R¹ is methyl.

57. The method of any one of claims 4 and 7-56, wherein R² is optionally substituted C₁₋₆ alkyl.

58. The method of any one of claims 4 and 7-57, wherein R² is unsubstituted C₁₋₆ alkyl.

59. The method of any one of claims 4 and 7-58, wherein R² is methyl.

60. The method of any one of claims 4 and 7-59, wherein R³ is optionally substituted C₁₋₆ alkyl.

61. The method of any one of claims 4 and 7-60, wherein R³ is unsubstituted C₁₋₆ alkyl.

62. The method of any one of claims 4 and 7-61, wherein R³ is methyl.

63. The method of any one of claims 4 and 7-61, wherein R⁵ is optionally substituted C₁₋₆ alkyl.

64. The method of any one of claims 4 and 7-61, wherein R⁵ is unsubstituted C₁₋₆ alkyl.

65. The method of any one of claims 4 and 7-64, wherein R⁵ is methyl.

66. The method of any one of claims 4 and 7-65, wherein two R⁴ groups are taken together to form: 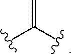
.

67. The method of any one of claims 4 and 7-66, wherein two R⁶ groups are taken together to form: 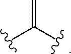
.

68. The method of any one of claims 4 and 7-67, wherein R^{X} and R^{Y} are both hydrogen.

69. The method of any one of claims 4 and 7-68, wherein R^{X} is hydrogen; and R^{Y} is –OR^{Ya}.

70. The method of any one of claims 4 and 7-69, wherein R^{X} is –OR^{Xa}; and R^{Y} is –OR^{Ya}.

71. The method of any one of claims 4, 5, and 7-70, wherein R^{P4} is an oxygen protecting group.

72. The method of any one of claims 4, 5, and 7-71, wherein R^{P4} is a silyl protecting group.

73. The method of any one of claims 4, 5, and 7-72, wherein R^{P4} is TES.

74. The method of any one of claims 4, 5, and 7-73, wherein R^{P5} is an oxygen protecting group.

75. The method of any one of claims 4, 5, and 7-74, wherein R^{P5} is a silyl protecting group.

76. The method of any one of claims 4, 5, and 7-75, wherein R^{P5} is TES.

77. The method of any one of claims 4, 5, and 7-76, wherein two R^{P6} are joined together with the intervening atoms to form: 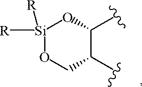
wherein each instance of R is independently optionally substituted alkyl, optionally substituted alkenyl, optionally substituted alkynyl, optionally substituted aryl, optionally substituted carbocyclyl, optionally substituted heteroaryl, or optionally substituted heterocyclyl, or optionally substituted hydroxyl.

78. The method of any one of claims 4, 5, and 7-77, wherein two R^{P6} are joined together with the intervening atoms to form: 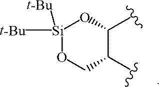
.
